(19) United States
(12) Patent Application Publication
(10) Pub. No.: US 2007/0238718 A1
(43) Pub. Date: Oct. 11, 2007

(54) THIAZOLYL-DIHYDRO-INDAZOLE

(76) Inventors: Matthias GRAUERT, Biberach (DE); Udo Maier, Senden (DE); Matthias Hoffmann, Mittelbiberach (DE); Stefan Scheuerer, Warthausen (DE); Anne T. Joergensen, Biberach (DE); Alexander Pautsch, Ulm (DE); Trixi Brandl, Basel (CH); Christoph Hoenke, Ingelheim (DE); Steffen Breitfelder, Assmannshardt (DE); Klaus Erb, Mittelbiberach (DE); Michael Pieper, Biberach (DE); Ingo Pragst, Muenchen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/690,351

(22) Filed: Mar. 23, 2007

(30) Foreign Application Priority Data
Apr. 6, 2006 (EP) 06112297

(51) Int. Cl. A61K 31/55; A61K 31/4747; A61K 31/43; C07D 498/02

(52) U.S. Cl. 514/210.16; 514/366; 514/212.02; 514/278; 546/16; 540/543; 548/147; 548/151

(57) ABSTRACT

The present invention relates to new thiazolyl-dihydro-indazoles of general formula (I) 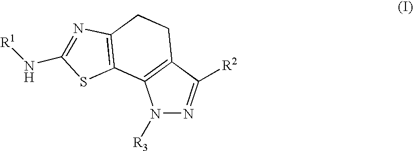
wherein the groups R¹, R² and R³ have the meanings given in the claims and specification, the tautomers, racemates, enantiomers, diastereomers and the mixtures thereof, and optionally the pharmacologically acceptable acid addition salts, solvates and hydrates thereof, and processes for preparing these thiazolyl-dihydro-indazoles and the use thereof as pharmaceutical compositions.

This application claims priority benefit to EP 06112297, filed Apr. 6, 2006, the entirety of which is incorporated herein.

The present invention relates to new thiazolyl-dihydro-indazoles of general formula (I) 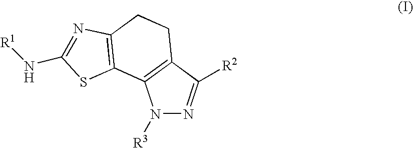
wherein the groups R¹, R² and R³ have the meanings given in the claims and specification, the tautomers, racemates, enantiomers, diastereomers and the mixtures thereof, and optionally the pharmacologically acceptable acid addition salts, solvates and hydrates thereof, and processes for preparing these thiazolyl-dihydro-indazoles and the use thereof as pharmaceutical compositions.

### BACKGROUND TO THE INVENTION

Phosphatidylinositol-3-kinases (PI3-kinases) are a subfamily of the lipid kinases which catalyse the transfer of a phosphate group to the 3′-position of the inositol ring of phosphoinositides.

They have a role in numerous cell processes such as e.g. cell growth and differentiation processes, the control of cytoskeletal changes and the regulation of intracellular transport processes (Vanhaesebroeck et al., Annu Rev Biochem. 2001; 70:535-602).

PI3-kinases may play a part in numerous tumours, such as e.g. breast cancer, ovarian or pancreatic carcinoma, in tumour types such as carcinomas of the colon, breast or lungs, but particularly in autoimmune diseases such as Crohn's disease or rheumatoid arthritis, for example, or in the cardiovascular system, e.g. in the development of cardiac hypertrophy (Oudit et al., Circulation. 2003 Oct. 28; 108(17):2147-52). PI3-kinase modulators may represent a possible method of anti-inflammatory therapy with comparatively minor side effects (Ward and Finan, Curr Opin Pharmacol. 2003 August; 3(4):426-34).

PI3-kinase inhibitors for treating inflammatory diseases are known in the literature. Thus, WO 03/072557 discloses 5-phenylthiazole derivatives, WO 04/029055 discloses annelated azolpyrimidines and WO 04/007491 discloses azolidinone-vinyl linked benzene derivatives. Moreover, the two specifications WO 04/052373 and WO 04/056820 disclose benzoxazine and benzoxazin-3-one derivatives.

The aim of the present invention is to provide new compounds which by virtue of their pharmaceutical activity as PI3-kinase modulators may be used therapeutically for the treatment of inflammatory or allergic diseases. Examples of these include inflammatory and allergic respiratory complaints, inflammatory and allergic skin complaints, inflammatory eye diseases, diseases of the nasal mucosa, inflammatory or allergic illnesses which involve autoimmune reactions or kidney inflammation.

### DETAILED DESCRIPTION OF THE INVENTION

Surprisingly it has been found that the above problem is solved by means of compounds of formula (I), wherein the groups R¹ to R³ have the meanings given hereinafter.

It has particularly been found that compounds of formula (I) act as inhibitors of PI3-kinase, particularly as inhibitors of PI3-kinase gamma. Thus the compounds according to the invention may be used for example for the treatment of respiratory complaints.

The present invention therefore relates to compounds of general formula (I), 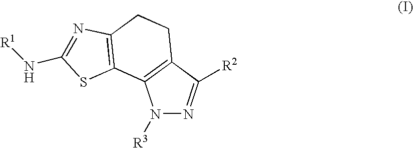
wherein
R¹ denotes hydrogen, CO—CH₃, CO—CH₂—R⁴, CO—CHMe-R⁴, CO—OR⁴, CO—SR⁴, CO—NH₂ or CO—NHR⁴;
R² denotes a group selected from among C₃₋₆-cycloalkyl, C₁₋₄-alkyl-C₃₋₆-cycloalkyl, C₂₋₄-alkenyl-C₃₋₆-cycloalkyl, C₂₋₄-alkynyl-C₃₋₆-cycloalkyl, C₅₋₆-cycloalkenyl, C₁₋₆-alkyl-C₅₋₆-cycloalkenyl, C₂₋₄-alkenyl-C₅₋₆-cycloalkenyl, C₂₋₄-alkynyl-C₅₋₆-cycloalkenyl, C₅₋₆-cycloalkynyl, C₁₋₆-alkyl-C₅₋₆-cycloalkynyl, C₂₋₄-alkenyl-C₅₋₆-cycloalkynyl- and C₂₋₄-alkynyl-C₅₋₆-cycloalkynyl, which may optionally be substituted by one or two of the groups CH₃, F, OCH₃, OH or NH₂;
R³ denotes a group selected from among C₆-C₁₄-aryl, C₁₋₆-alkyl-C₆-C₁₄-aryl, C₂₋₆-alkenyl-C₆-C₁₄-aryl, C₂₋₆-alkynyl-C₆-C₁₄-aryl, C₅-C₁₀-heteroaryl, C₁₋₁₂-alkyl-C₅-C₁₀-heteroaryl, C₃₋₁₂-alkenyl-C₅-C₁₀-heteroaryl, C₃₋₁₂-alkynyl-C₅-C₁₀-heteroaryl, C₃₋₆-cycloalkyl, C₁₋₆-alkyl-C₃₋₆-cycloalkyl, C₂₋₄-alkenyl-C₃₋₆-cycloalkyl, C₂₋₄-alkynyl-C₃₋₆-cycloalkyl, C₅₋₆-cycloalkenyl, C₁₋₆-alkyl-C₅₋₆-cycloalkenyl, C₂₋₄-alkenyl-C₅₋₆-cycloalkenyl, C₂₋₄-alkynyl-C₅₋₆-cycloalkenyl, C₅₋₆-cycloalkynyl, C₁₋₆-alkyl-C₅₋₆-cycloalkynyl, C₂₋₄-alkenyl-C₅₋₆-cycloalkynyl- and C₂₋₄-alkynyl-C₅₋₆-cycloalkynyl, which may optionally be substituted by a group R⁵ and up to three groups R⁶;
   or optionally substituted 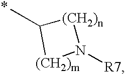
   wherein
   n, m, independently of one another denote 1 or 2;
R⁴ denotes an optionally substituted group selected from among C₁₋₄-alkyl, C₂₋₁₀-alkenyl, C₂₋₁₀-alkynyl, C₃₋₆-cycloalkyl-C₁₋₄-alkyl, C₃₋₆-cycloalkyl-C₃₋₁₀-alkenyl, C₃₋₆-cycloalkyl-C₃₋₁₀-alkynyl, C₆-C₁₄-aryl, C₆-C₁₄-aryl-C₁₋₄-alkyl, C₅-C₁₀-heteroaryl, C₅-C₁₀-heteroaryl-C₁₋₄-alkyl- and haloalkyl;
R⁵ denotes CONR⁸R⁹, NR⁸COR⁹, NR⁸R⁹, OR⁹ and —C₁₋₄-alkyl-CONR⁸R⁹;
R⁶ which may be identical or different, denote F, Cl, Br, OH, CN, CF₃, CHF₂ or an optionally substituted group selected from among O—C₁₋₃-alkyl, O—C₃₋₄-alkenyl, O—C₃₋₄-alkynyl, C₁₋₃-alkyl, C₂₋₆-alkenyl and C₂₋₃-alkynyl, C₃₋₆-cycloalkyl-C₁₋₄-alkyl, C₃₋₆-cycloalkyl-C₂₋₄-alkenyl, C₃₋₆-cycloalkyl-C₂₋₄-alkynyl, C₅₋₆-cycloalkenyl-C₁₋₄-alkyl, C₅₋₆-cycloalkenyl-C₃₋₁₀-alkenyl, C₅₋₆-cycloalkenyl-C₂₋₄-alkynyl, C₆-C₁₄-aryl-C₁₋₁₄-alkyl, C₆-C₁₄-aryl-C₂₋₄-alkenyl, C₆-C₁₄-aryl-C₂₋₄-alkynyl, C₅-C₁₀-heteroaryl-C₁₋₄-alkyl, C₅-C₁₀-heteroaryl-C₂₋₄-alkenyl- and C₅-C₁₀-heteroaryl-C₂₋₄-alkynyl,
R⁷ denotes hydrogen, COR⁹, CONR⁸R⁹ or
   a group selected from among C₁₋₁₀-alkyl, C₃₋₁₀-alkenyl, C₃₋₁₀-alkynyl, C₃₋₆-cycloalkyl-C₁₋₁₄-alkyl, C₃₋₆-cycloalkyl-C₃₋₁₀-alkenyl, C₃₋₆-cycloalkyl-C₃₋₁₀-alkynyl, C₅₋₆-cycloalkenyl-C₁₋₄-alkyl, C₅₋₆-cycloalkenyl-C₃₋₁₀-alkenyl, C₅₋₆-cycloalkenyl-C₃₋₁₀-alkynyl, C₆-C₁₄-aryl, C₁₋₁₀-alkyl-C₆-C₁₄-aryl, C₂₋₁₀-alkenyl-C₆-C₁₄-aryl-, C₂₋₁₀-alkynyl-C₆-C₁₄-aryl, C₅-C₁₀-heteroaryl, C₁-12-alkyl-C₅-C₁₀-heteroaryl, C₃₋₁₂-alkenyl-C₅-C₁₀-heteroaryl- and C₃₋₁₂-alkynyl-C₅-C₁₀-heteroaryl, which may optionally be substituted by a group R¹⁴ and by a group R¹³;
R⁸ denotes hydrogen or
   an optionally substituted group selected from among C₁₋₁₀-alkyl, C₃₋₁₀-alkenyl, C₃₋₁₀-alkynyl, C₃₋₆-cycloalkyl-C₁₋₄-alkyl, C₃₋₆-cycloalkyl-C₃₋₁₀-alkenyl, C₃₋₆-cycloalkyl-C₃₋₁₀-alkynyl, C₅₋₆-cycloalkenyl-C₁₋₄-alkyl, C₅₋₆-cycloalkenyl-C₃₋₁₀-alkenyl, C₅₋₆-cycloalkenyl-C₃₋₁₀-alkynyl, C₆-C₁₄-aryl-C₁₋₄-alkyl, C₆-C₁₄-aryl-C₃₋₁₀-alkenyl- and C₆-C₁₄-aryl-C₃₋₁₀-alkynyl, C₅-C₁₀-heteroaryl, C₅-C₁₀-heteroaryl-C₁₋₄-alkyl, C₅-C₁₀-heteroaryl-C₁₋₄-alkenyl, C₅-C₁₀-heteroaryl-C₁₋₄-alkynyl, C₁₋₄-alkyl-O—C₂₋₄-alkyl, C₁₋₄-alkyl-O—C₄₋₆-alkenyl- and C₁₋₄-alkyl-O—C₄₋₆-alkynyl-;
R⁹ denotes hydrogen or
   an optionally substituted group selected from among C₁₋₁₂-alkyl, C₃₋₁₂-alkenyl, C₃₋₁₂-alkynyl, C₃₋₆-cycloalkyl-C₁₋₁₂-alkyl, C₃₋₆-cycloalkyl-C₃₋₁₂-alkenyl, C₃₋₆-cycloalkyl-C₃₋₁₂-alkynyl, C₅₋₆-cycloalkenyl-C₁₋₄-alkyl, C₅₋₆-cycloalkenyl-C₃₋₁₀-alkenyl, C₅₋₆-cycloalkenyl-C₃₋₁₀-alkynyl, C₆-C₁₄-aryl-C₁₋₁₂-alkyl, C₆-C₁₄-aryl-C₃₋₁₂-alkenyl, C₆-C₁₄-aryl-C₃₋₁₂-alkynyl, C₆-C₁₄-aryl, C₁₋₁₂-alkyl-C₆-C₁₄-aryl, C₂₋₁₂-alkenyl-C₆-C₁₄-aryl, C₂₋₁₂-alkynyl-C₆-C₁₄-aryl, C₅-C₁₀-heteroaryl, C₅-C₁₀-heteroaryl-C₁₋₁₂-alkyl, C₅-C₁₀-heteroaryl-C₃₋₁₂-alkenyl, C₅-C₁₀-heteroaryl-C₃₋₁₂-alkynyl, C₃₋₈-cycloalkyl, C₅₋₈-cycloalkenyl, NR¹¹R¹²—C₃₋₈-cycloalkyl, NR¹¹R¹²—C₅₋₈-cycloalkenyl- and NR¹¹R¹²—C₅₋₈-cycloalkynyl or
   an optionally substituted C₃₋₈-heterocycloalkyl-(CH₂)_{q} group, containing at least one NR¹⁰ group in the 3- to 8-membered heterocyclic group, or
R⁸ and R⁹ together form a saturated or unsaturated 4- to 7-membered alkyl bridge which optionally contains an O atom or an S(O)ₚ group,
   wherein p, q independently of one another denote 0, 1 or 2; or
NR⁸R⁹ denotes a 5- to 6-membered heterocyclic group, optionally containing a further N atom and optionally substituted by a group selected from among R¹⁰, NR¹¹R¹² and NR¹¹R¹²C₁₋₄-alkyl, or
   a group 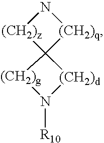
   wherein
   z, q, g, d independently of one another denote 1, 2 or 3;
R¹⁰ denotes hydrogen or
   an optionally substituted group selected from among C₁₋₁₀-alkyl, C₃₋₁₀-alkenyl, C₃₋₁₀-alkynyl, C₃₋₇-cycloalkyl-C₁₋₁₀-alkyl, C₃₋₇-cycloalkyl-C₃₋₁₀-alkenyl, C₃₋₇-cycloalkyl-C₃₋₁₀-alkynyl, C₃₋₇-cycloalkyl, C₁₋₆-alkyl-C₃₋₇-cycloalkyl, C₂₋₄-alkenyl-C₃₋₇-cycloalkyl, C₂₋₄-alkynyl-C₃₋₇-cycloalkyl, tetrahydropyranyl and (NR⁴)₂CH—C₁₋₁₀-alkyl,
R¹¹, R¹² which may be identical or different denote hydrogen or
   an optionally substituted group selected from among C₁₋₁₀-alkyl, C₃₋₁₀-alkenyl, C₃₋₁₀-alkynyl, C₃₋₆-cycloalkyl-C₁₋₄-alkyl- and C₃₋₆-cycloalkyl or
R¹¹ and R¹² together form a 4- to 7-membered alkyl chain which optionally contains a heteroatom;
R¹³ denotes F, Cl, Br, OH, CN, CF₃, CHF₂ or C₁₄-alkyl-O,
R¹⁴ denotes NR¹¹R¹² or an optionally substituted C₃₋₈-heterocycloalkyl-(CH₂)_{q} group, containing at least one NR¹⁰ group in the 3- to 8-membered heterocyclic group, or
R¹³ and R¹⁴ together form a saturated or unsaturated 4- to 7-membered alkyl bridge which optionally contains an O atom or a S(O)ₚ group,
   optionally in the form of the tautomers, the racemates, the enantiomers, the diastereomers and the mixtures thereof, as well as optionally the pharmacologically acceptable acid addition salts, solvates and hydrates thereof.

Preferred are compounds of formula (I), wherein
R¹ and R³ to R¹⁴ may have the meanings specified and
R² denotes a group selected from among C₃₋₆-cycloalkyl, C₁₋₆-alkyl-C₃₋₆-cycloalkyl- and C₂₋₄-alkenyl-C₃₋₆-cycloalkyl, which may optionally be substituted by one or two of the groups CH₃, F, OCH₃, OH or NH₂.

Also preferred are compounds of formula (I), wherein
R¹, R² and R⁴ to R¹⁴ may have the meanings specified and
R³ denotes a group selected from among phenyl and C₅₋₆-cycloalkyl, which may optionally be substituted by a group R⁵ and up to three groups R⁶,
   or optionally substituted 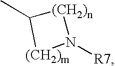
   wherein
   n, m, independently of one another denote 1 or 2.

Also preferred are compounds of formula (I), wherein
R¹ to R⁷ and R¹⁰ to R¹⁴ may have the meanings specified and
R⁸ denotes hydrogen or
   an optionally substituted group selected from among C₁₋₁₀-alkyl, C₃₋₁₀-alkenyl, C₃₋₁₀-alkynyl and C₁₋₄-alkyl-O—C₂₋₄-alkyl,
R⁹ denotes hydrogen or
   an optionally substituted group selected from among C₁₋₁₂-alkyl, C₃₋₁₂-alkenyl, C₃₋₁₂-alkynyl, C₃₋₆-cycloalkyl-C₁₋₁₂-alkyl, C₆-C₁₄-aryl, C₁₋₁₂-alkyl-C₆-C₁₄-aryl, C₂₋₁₂-alkenyl-C₆-C₁₄-aryl, C₂₋₁₂-alkynyl-C₆-C₁₄-aryl, C₅-C₁₀-heteroaryl, C₅-C₁₀-heteroaryl-C₁₋₁₂-alkyl, heteroaryl-C₃₋₁₂-alkenyl, heteroaryl-C₃₋₁₂-alkynyl, C₃₋₈-cycloalkyl, C₅₋₈-cycloalkenyl and NR¹¹R¹²—C₃₋₈-cycloalkyl, or
   an optionally substituted C₃₋₈-heterocycloalkyl-(CH₂)_{q}— containing at least one NR¹⁰ group in the 3- to 8-membered heterocyclic group, or
R⁸ and R⁹ together form a saturated or unsaturated 4- to 7-membered alkyl bridge which optionally contains an O atom or a S(O)ₚ group,
   wherein p, q independently of one another denote 0, 1 or 2, or
NR⁸R⁹ denotes a 5- to 6-membered heterocyclic group, optionally containing a further N atom and optionally substituted by a group selected from among R¹⁰, NR¹¹R¹² and NR¹¹R¹²C₁₋₄-alkyl, or
   a group 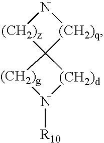
   wherein
   z, q, g, d independently of one another denote 1, 2 or 3.

Also preferred are compounds of formula (1), wherein
R¹ to R⁷ and R¹⁰ to R¹⁴ may have the meanings specified and
R⁸ denotes hydrogen or
   an optionally substituted group selected from among C₁₋₁₀-alkyl, C₃₋₁₀-alkenyl, C₃₋₁₀-alkynyl and C₁₋₄-alkyl-O—C₁₋₄-alkyl,
R⁹ denotes hydrogen or
   an optionally substituted group selected from among C₁₋₁₂-alkyl, C₃₋₁₂-alkenyl, C₃₋₁₂-alkynyl, C₃₋₆-cycloalkyl-C₁₋₁₂-alkyl, C₆-C₁₄-aryl, C₁₋₁₂-alkyl-C₆-C₁₄-aryl, C₂₋₁₂-alkenyl-C₆-C₁₄-aryl, C₂₋₁₂-alkynyl-C₆-C₁₄-aryl, C₅-C₁₀-heteroaryl, C₅-C₁₀-heteroaryl-C₁₋₁₂-alkyl, C₅-C₁₀-heteroaryl-C₃₋₁₂-alkenyl, C₅-C₁₀-heteroaryl-C₃₋₁₂-alkynyl, C₃₋₈-cycloalkyl, C₅₋₈-cycloalkenyl and NR¹¹R¹²—C₃₋₈-cycloalkyl, or
   an optionally substituted group selected from among the general formulae (A1) to (A12) 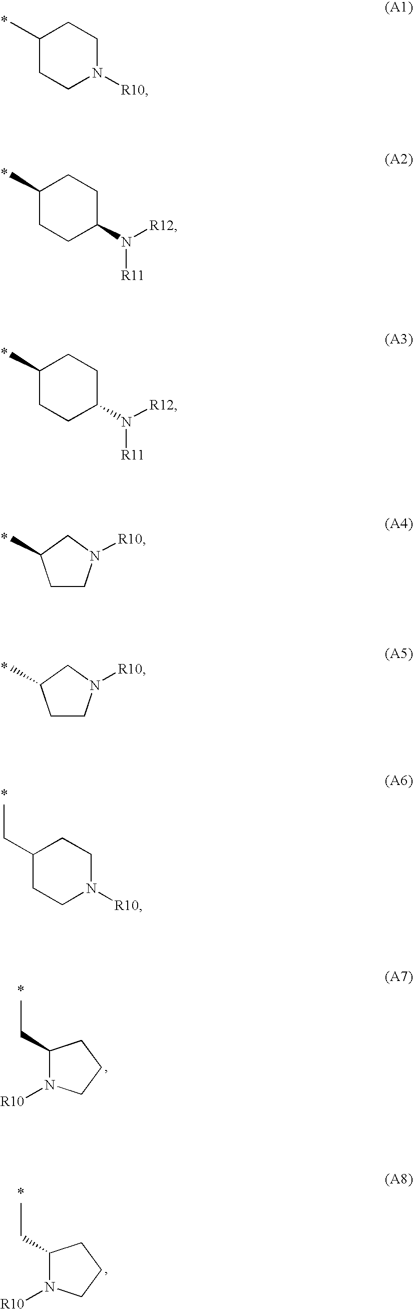 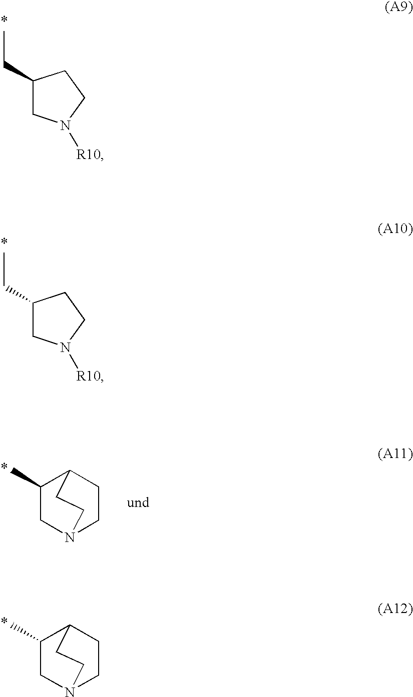
      or
R⁸ and R⁹ together form a saturated or unsaturated 4- to 7-membered alkyl bridge which optionally contains an O atom or a S(O)ₚ group,
   wherein p, q independently of one another denote 0, 1 or 2; or
NR⁸R⁹ an optionally substituted group selected from among the general formulae (B1) to (B8) 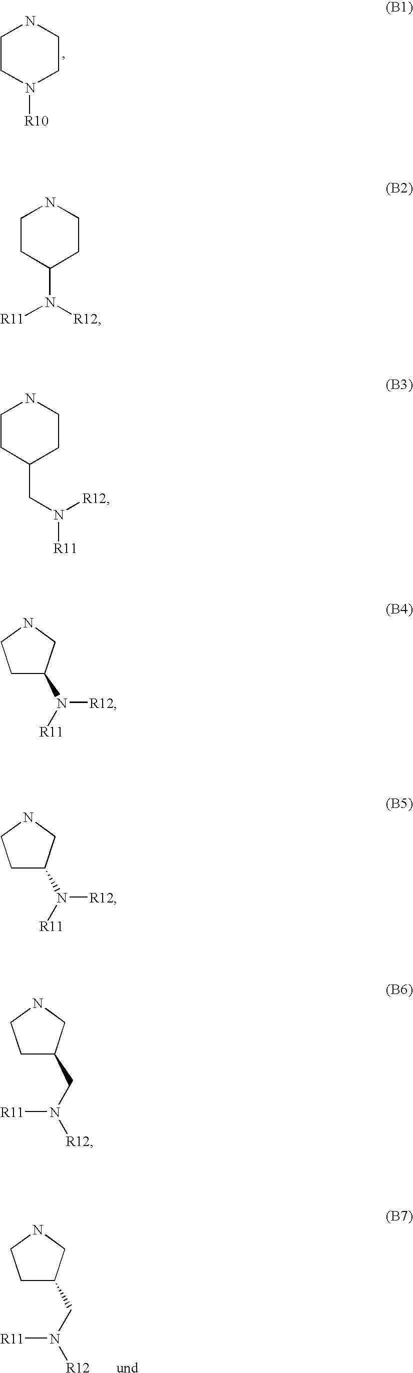 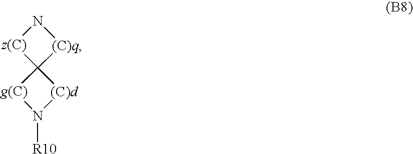
   wherein
   z, q, g, d independently of one another denote 1, 2 or 3.

Also preferred are compounds of formula (I), wherein
R¹ to R⁸ and R¹⁰ to R¹² may have the meanings specified and
R⁷ denotes COR⁹ or CONR⁸R⁹.

Also preferred are compounds of formula (I), wherein
R¹ to R⁵ and R⁷ to R¹⁴ may have the meanings specified and
R⁶ which may be identical or different, denote F, Cl, CF₃, or an optionally substituted group O—C₁₋₃-alkyl or C₁₋₃-alkyl.

Also preferred are compounds of formula (I), wherein
R⁴ to R⁵ and R¹⁰ to R¹² may have the meanings specified and
R¹ denotes CO—CH₃, CO—CH₂—R⁴
R² denotes cyclopropyl, which may optionally be substituted by one or two of the groups CH₃, F, OCH₃, OH or NH₂,
R³ denotes optionally substituted 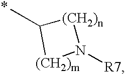
   wherein
   n, m, independently of one another denote 1 or 2,
R⁷ denotes hydrogen, COR⁹, or CONR⁸R⁹,
R⁸ denotes hydrogen or C₁₋₁₀-alkyl,
R⁹ denotes hydrogen or
   an optionally substituted group selected from among C₃₋₈-cycloalkyl and NR¹¹ R¹²—C₃₋₈-cycloalkyl, or
   an optionally substituted group selected from among the general formulae (A1) to (A12) 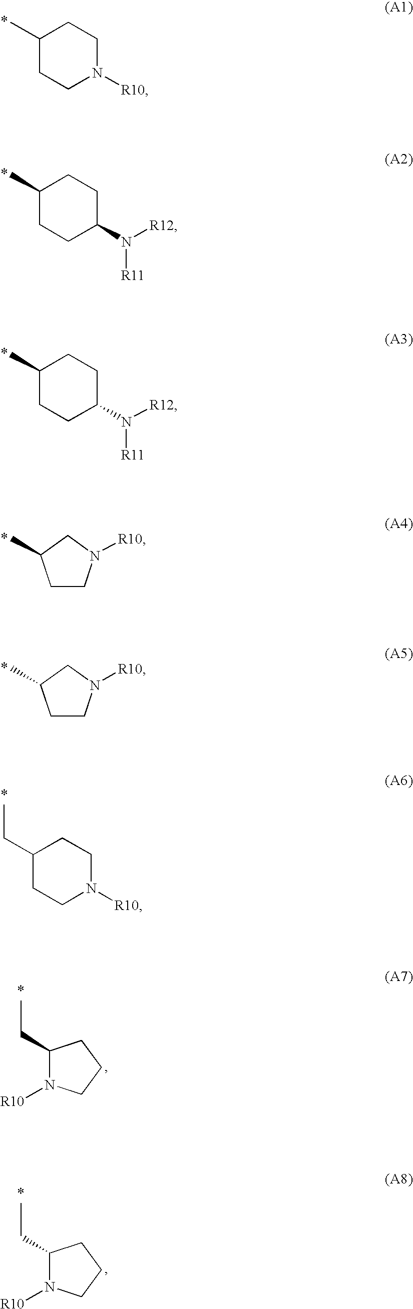 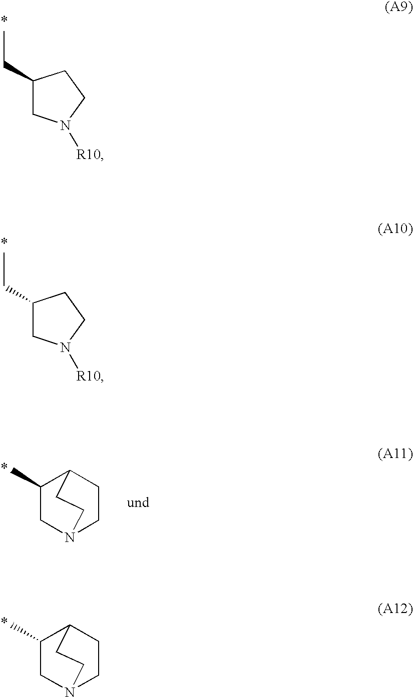
      or
NR⁸R⁹ denotes a 5- to 6-membered heterocyclic group, containing 1 to 3 N— atoms, which may optionally be substituted by a group selected from among R¹⁰, NR¹¹R¹² and NR¹¹R¹²C₁₋₄-alkyl.

Particularly preferred are compounds of formula (I), wherein
R⁴ to R⁶ and R¹⁰ to R¹² may have the meanings specified and
R¹ denotes CO—CH₃, CO—CH₂—R⁴;
R² denotes C₃₋₆-cycloalkyl, which may optionally be substituted by one or two of the groups CH₃, F, OCH₃, OH or NH₂;
R³ denotes a group selected from among phenyl and C₅₋₆-cycloalkyl, which may optionally be substituted by a R⁵ and up to three R⁶,
R⁵ denotes NR⁸R⁹ CONR⁸R⁹, NR⁸COR⁹ or —C₁₋₄-alkyl-CONR⁸R⁹,
R⁶ which may be identical or different, denote F, Cl, Br, CF₃ or an optionally substituted group selected from among O—C₁₋₃-alkyl, C₁₋₃-alkyl, C₃₋₆-cycloalkyl-C₁₋₄-alkyl and C₆-C₁₄-aryl-C₁₋₄-alkyl,
R⁸ denotes hydrogen or
   optionally substituted C₁₋₁₀-alkyl,
R⁹ denotes hydrogen or
   an optionally substituted group selected from among C₁₋₁₂-alkyl, C₃₋₆-cycloalkyl-C₁₋₁₂-alkyl, C₆-C₁₄-aryl, C₁₋₁₂-alkyl-C₆-C₁₄-aryl, C₅-C₁₀-heteroaryl, C₅-C₁₀-heteroaryl-C₁₋₁₂-alkyl, C₃₋₈-cycloalkyl, C₅₋₈-cycloalkenyl and NR¹¹R¹²—C₃₋₈-cycloalkyl, or
   an optionally substituted group selected from among the general formulae (A1) to (A12) 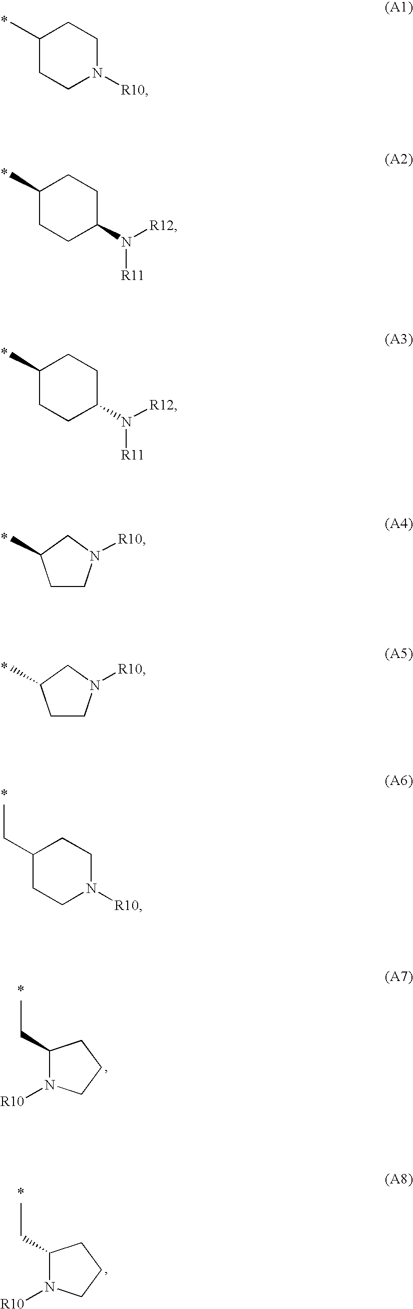 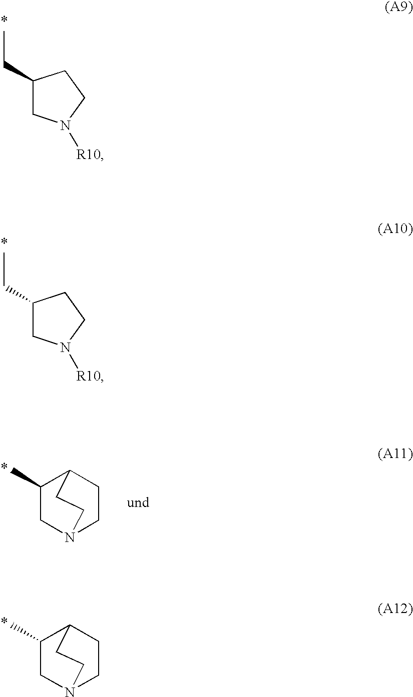
R⁸ and R⁹together form a saturated or unsaturated 4- to 7-membered alkyl bridge which optionally contains an O atom or a S(O)ₚ group,
   wherein p, q independently of one another denote 0, 1 or 2; or
NR⁸R⁹ denotes an optionally substituted group selected from among the general formulae (B1) to (B8) 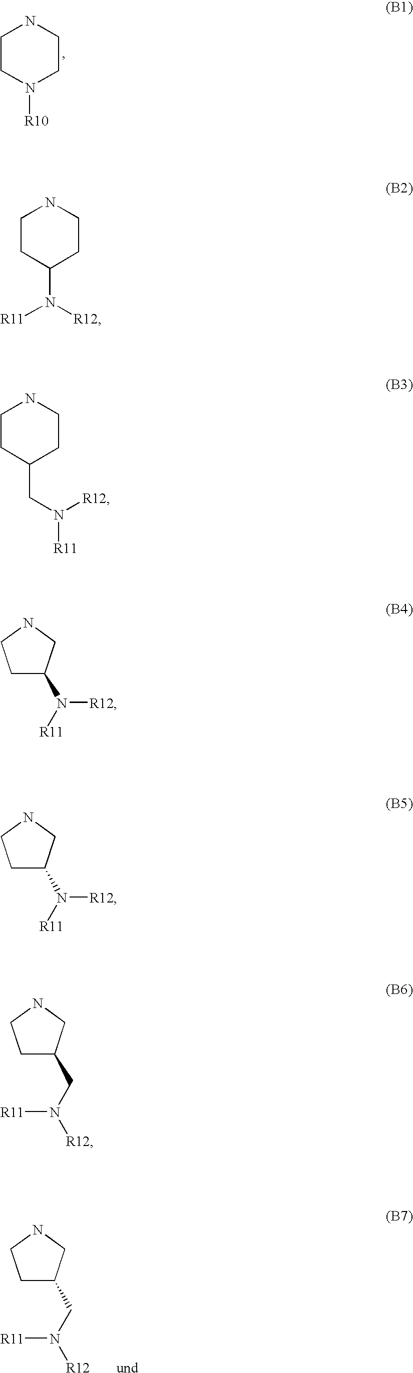 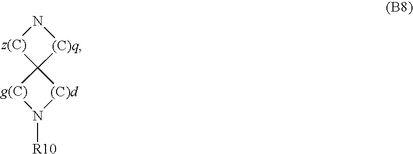
   wherein
   z, q, g, d independently of one another denote 1, 2 or 3.
R¹⁰ denotes hydrogen or
   an optionally substituted group selected from among C₁₋₁₀-alkyl, C₃₋₇-cycloalkyl-C₁₋₁₀-alkyl, C₃₋₇-cycloalkyl, C₁₋₆-alkyl-C₃₋₇-cycloalkyl, tetrahydropyranyl and (NR⁴)₂CH—C₁₋₁₀-alkyl.

In another aspect the invention relates to compounds of formula (I) for use as pharmaceutical compositions.

The invention further relates to the use of the compounds of formula (I) for preparing a pharmaceutical composition for the treatment of diseases in whose pathology an activity of PI3-kinases is implicated, wherein therapeutically effective doses of the compounds of formula (I) may confer a therapeutic benefit.

The invention further relates to the use of the compounds of formula (I), for preparing a pharmaceutical composition for the treatment of inflammatory and allergic diseases of the airways.

The invention further relates to the use of the compounds of formula (I), for preparing a pharmaceutical composition for the treatment of a disease, which is selected from among chronic bronchitis, bronchitis caused by bacterial or viral infections or fungi or helminths, allergic bronchitis, toxic bronchitis, chronic obstructive bronchitis (COPD), asthma (intrinsic or allergic), paediatric asthma, bronchiectases, allergic alveolitis, allergic or non-allergic rhinitis, chronic sinusitis, cystic fibrosis or mucoviscidosis, alphal-antitrypsin deficiency, coughing, pulmonary emphysema, interstitial lung diseases, alveolitis, hyperreactive airways, nasal polyps, pulmonary oedema, pneumonitis of various causes, such as radiation-induced or caused by aspiration or infection, collagenoses such as lupus erythematodes, systemic scleroderma, sarcoidosis and Boeck's disease.

The invention further relates to the use of the compounds of formula (I), for preparing a pharmaceutical composition for the treatment of inflammatory and allergic diseases of the skin.

The invention further relates to the use of the compounds of formula (I), for preparing a pharmaceutical composition for the treatment of a disease which is selected from among psoriasis, contact dermatitis, atopical dermatitis, alopecia areata (circular hair loss), erythema exsudativum multiforme (Stevens-Johnson Syndrome), dermatitis herpetiformis, sclerodermy, vitiligo, nettle rash (urticaria), lupus erythematodes, follicular and surface pyoderma, endogenous and exogenous acne, acne rosacea and other inflammatory and allergic or proliferative skin complaints.

The invention further relates to the use of the compounds of formula (I), for preparing a pharmaceutical composition for the treatment of inflammation of the eye.

The invention further relates to the use of the compounds of formula (I), for preparing a pharmaceutical composition for the treatment a disease which is selected from among conjunctivitis of various kinds, such as e.g. caused by fungal or bacterial infections, allergic conjunctivitis, irritable conjunctivitis, conjunctivitis caused by drugs, keratitis and uveitis.

The invention further relates to the use of the compounds of formula (I), for preparing a pharmaceutical composition for the treatment of diseases of the nasal mucosa.

The invention further relates to the use of the compounds of formula (I), for preparing a pharmaceutical composition for the treatment of a disease, which is selected from among allergic rhinitis, allergic sinusitis and nasal polyps.

The invention further relates to the use of the compounds of formula (I), for preparing a pharmaceutical composition for the treatment of inflammatory or allergic conditions involving autoimmune reactions.

The invention further relates to the use of the compounds of formula (I), for preparing a pharmaceutical composition for the treatment of a disease which is selected from among Crohn's disease, ulcerative colitis, systemic lupus erythematodes, chronic hepatitis, multiple sclerosis, rheumatoid arthritis, psoriatric arthritis, osteoarthritis, rheumatoid spondylitis.

The invention further relates to the use of the compounds of formula (I), for preparing a pharmaceutical composition for the treatment of kidney inflammation.

The invention further relates to the use of the compounds of formula (I), for preparing a pharmaceutical composition for the treatment of a disease which is selected from among glomerulonephritis, interstitial nephritis and idiopathic nephrotic syndrome.

Of particular importance according to the invention is a pharmaceutical formulation containing a compound of formula (I).

Preferred is an inhaled pharmaceutical formulation containing a compound of formula (I).

Also preferred is an orally administered pharmaceutical formulation containing a compound of formula (I).

The invention further relates to compounds of general formula (VI) 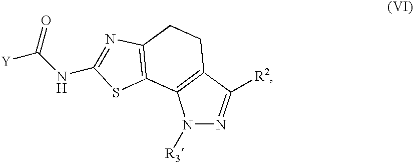
wherein R² and Y may have the meanings specified, and
R₃′ denotes an optionally substituted group, selected from among 4-PhCOOMe, 4-PhNO₂ and 4-piperidyl, cis/trans-4-alkoxycarbonylcylohexyl, 4-methoxycarbonyl-methy-phenyl,
optionally in the form of the tautomers, the racemates, the enantiomers, the diastereomers and the mixtures thereof, as well as optionally the pharmacologically acceptable acid addition salts thereof.

The invention further relates to compounds of general formula (IX) 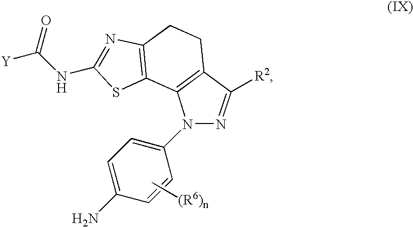
wherein R², R⁶ and Y may have the meanings specified,
optionally in the form of the tautomers, the racemates, the enantiomers, the diastereomers and the mixtures thereof, as well as optionally the pharmacologically acceptable acid addition salts thereof.

The invention further relates to compounds of general formula (VII) 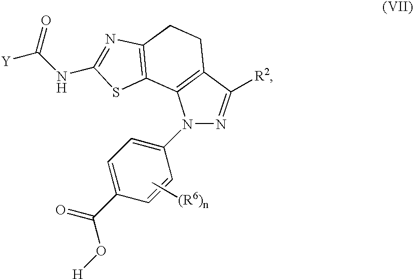
wherein R², R⁶ and Y may have the meanings specified,
optionally in the form of the tautomers, the racemates, the enantiomers, the diastereomers and the mixtures thereof, as well as optionally the pharmacologically acceptable acid addition salts thereof.
   Terms and Definitions Used

By alkyl groups as well as alkyl groups which are part of other groups are meant branched and unbranched alkyl groups with 1 to 10 carbon atoms, preferably 1-6, particularly preferably 1-4 carbon atoms, are meant for example: methyl, ethyl, propyl, butyl, pentyl, hexyl, heptyl, octyl, nonyl and decyl. Unless stated otherwise, the above terms propyl, butyl, pentyl, hexyl, heptyl, octyl, nonyl and decyl include all the possible isomeric forms. For example the term propyl includes the two isomeric groups n-propyl and iso-propyl, the term butyl includes n-butyl, iso-butyl, sec. butyl and tert.-butyl, the term pentyl includes isopentyl, neopentyl etc.

In the above-mentioned alkyl groups, unless otherwise specified, one or more hydrogen atoms may be replaced by other groups. For example these alkyl groups may be substituted by the halogen atoms fluorine, chlorine, bromine or iodine. The substituents fluorine or chlorine are preferred. It is also possible for all the hydrogen atoms of the alkyl group to be replaced.

By alkyl bridge is meant, unless stated otherwise, branched and unbranched double-bonded alkyl groups with 4 to 7 carbon atoms, for example, n-butylene, iso-butylene, sec. butylen and tert.-butylene, pentylene, iso-pentylene, neopentylene, etc. bridges. Particularly preferred are n-butylene or n-pentylene bridges. In the above-mentioned alkyl bridges 1 to 2 C atoms may optionally be replaced by one or more heteroatoms selected from among oxygen or sulphur.

Examples of alkenyl groups (including those which are part of other groups) are branched and unbranched alkenyl groups with 2 to 10 carbon atoms, preferably 2-6 carbon atoms, particularly preferably 2-3 carbon atoms, provided that they have at least one double bond. Examples include: ethenyl, propenyl, butenyl, pentenyl etc. Unless stated otherwise, the above terms propenyl, butenyl etc. include all the possible isomeric forms. For example the term butylene includes n-butenyl, 1-methylpropenyl, 2-methylpropenyl, 1,1-dimethylethenyl, 1,2-dimethylethenyl etc. In the above-mentioned alkenyl groups unless otherwise stated one or more hydrogen atoms may optionally be replaced by other groups. For example these alkenyl groups may be substituted by the halogen atoms fluorine, chlorine, bromine or iodine. The substituents fluorine and chlorine are preferred. Optionally, all the hydrogen atoms of the alkenyl group may be replaced.

Examples of alkynyl groups (including those which are part of other groups) include branched and unbranched alkynyl groups with 2 to 10 carbon atoms, provided that they have at least one triple bond, for example ethynyl, propargyl, butynyl, pentynyl, hexynyl etc., preferably ethynyl or propynyl.

Preferred are alkynyl groups with 2 to 4 carbon atoms. Examples of these include: ethynyl, propynyl, butynyl, pentynyl, or hexynyl. Unless stated otherwise, the definitions propynyl, butynyl, pentynyl and hexynyl include all the possible isomeric forms of the groups in question. Thus for example propynyl includes 1-propynyl and 2-propynyl, butynyl includes 1-, 2- and 3-butynyl, 1-methyl-1-propynyl, 1-methyl-2-propynyl etc.

In the above-mentioned alkynyl groups unless otherwise stated one or more hydrogen atoms may optionally be replaced by other groups. For example these alkyl groups may be substituted by the halogen atoms fluorine, chlorine, bromine or iodine. The substituents fluorine and chlorine are preferred. Optionally, all the hydrogen atoms of the alkynyl group may be replaced.

By cycloalkyl groups (including those which are part of other groups) are meant saturated cycloalkyl groups with 3-8 carbon atoms, for example cyclopropyl, cyclobutyl, cyclopentyl, cyclohexyl, cycloheptyl or cyclooctyl, preferably cyclopropyl, cyclopentyl or cyclohexyl, while each of the above-mentioned cycloalkyl groups may optionally carry one or more substituents or may be anellated to a benzene ring. Moreover the cycloalkyl groups may form, in addition to monocyclic ring systems, bicyclic, bridged or spirocyclic ring systems.

By cycloalkenyl (including those which are part of other groups) are meant cyclic alkyl groups with 5 to 8, preferably 5 or 6 carbon atoms, which contain one or two double bonds. Examples of these include: cyclopentenyl, cyclopentadienyl, cyclohexenyl, cyclohexadienyl, cycloheptenyl, cycloheptadienyl, cyclooctenyl or cyclooctadienyl. Moreover the cycloalkenyl groups may form, in addition to monocyclic ring systems, bicyclic, bridged or spirocyclic ring systems.

By cycloalkynyl (including those which are part of other groups) are meant cyclic alkyl groups with 5 to 8, preferably 5 or 6 carbon atoms, which contain one or two triple bonds. Examples of these include: cyclopentynyl, cyclopentadiynyl, cyclohexynyl, cyclohexadiynyl, cycloheptinyl, cycloheptadiynyl, cyclooctinyl or cyclooctadiynyl. Moreover the cycloalkynyl groups may form, in addition to monocyclic ring systems, bicyclic, bridged or spirocyclic ring systems.

By haloalkyl (including those which are part of other groups) are meant branched and unbranched alkyl groups with 1 to 6 carbon atoms, wherein one or more hydrogen atoms are replaced by a halogen atom selected from among fluorine, chlorine or bromine, preferably fluorine and chlorine. By the term “C₁₋₄-haloalkyl” are meant correspondingly branched and unbranched alkyl groups with 1 to 4 carbon atoms, wherein one or more hydrogen atoms are replaced as described above. C₁₋₄-haloalkyl is preferred. Examples of these include: CH₂F, CHF₂, CF₃.

The term denotes an aromatic ring system with 6 to 14 carbon atoms, preferably 6 or 10 carbon atoms, for example phenyl or naphthyl, preferably phenyl, which, unless otherwise described, may have one or more substituents, for example.

By heterocycloalkyl groups are meant, unless otherwise described in the definitions, 5-, 6- or 7-membered, saturated or unsaturated, mono- or bicyclic heterocycles, wherein up to four C atoms may be replaced by one or more heteroatoms selected from among oxygen, nitrogen or sulphur, for example tetrahydrofuran, tetrahydrofuranon, γ-butyrolactone, α-pyran, γ-pyran, dioxolan, tetrahydropyran, dioxane, dihydrothiophen, thiolan, dithiolan, pyrroline, pyrrolidine, pyrazoline, pyrazolidine, imidazoline, imidazolidine, tetrazole, piperidine, pyridazine, pyrimidine, pyrazine, piperazine, triazine, tetrazine, morpholine, thiomorpholine, diazepan, oxazine, tetrahydro-oxazinyl, isothiazole, pyrazolidine, preferably pyrazolyl, pyrrolidinyl, piperidinyl, piperazinyl or tetrahydro-oxazinyl, wherein the heterocyclic group may optionally be substituted. The ring may be linked to the molecule via a carbon atom or if available via a nitrogen atom.

Unless otherwise mentioned, a heterocyclic ring may be provided with a keto group. Examples of these include.

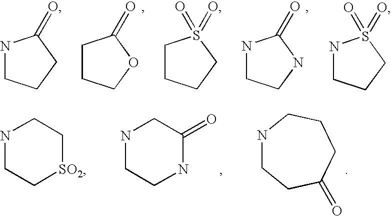

Examples of 5-10-membered bicyclic hetero rings include pyrrolizine, indole, indolizine, isoindole, indazole, purine, quinoline, isoquinoline, benzimidazole, benzofuran, benzopyran, benzothiazole, benzothiazole, benzisothiazole, pyridopyrimidine, pteridine, pyrimidopyrimidine,

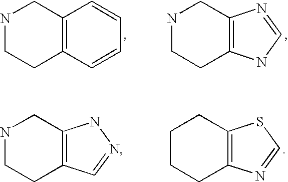

Examples of heteroaryl include 5-10-membered mono- or bicyclic heteroaryl rings in which up to three C atoms may be replaced by one or more heteroatoms selected from among oxygen, nitrogen or sulphur, while these may contain so many conjugated double bonds that an aromatic system is formed. Each of the above-mentioned heterocycles may optionally also be anellated to a benzene ring, preferably benzimidazole. The heteroaryl rings may, unless otherwise described, carry one or more substituents, for example.

The ring may be linked to the molecule through a carbon atom or if present through a nitrogen atom. The following are examples of five- or six-membered heterocyclic aromatic groups:

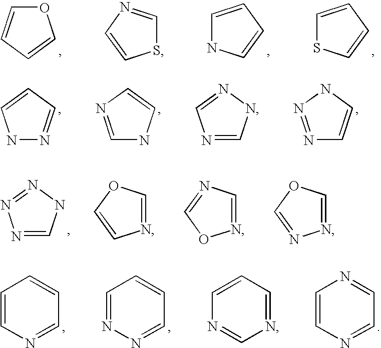

Examples of 5-10-membered bicyclic heteroaryl rings include pyrrolizine, indole, indolizine, isoindole, indazole, purine, quinoline, isoquinoline, benzimidazole, benzofuran, benzopyran, benzothiazole, benzoisothiazole, pyridopyrimidine, pteridine, pyrimidopyrimidine.

By the term heterocyclic spiro rings (“spiro”) are meant 5-10 membered, spirocyclic rings which may optionally contain one, two or three heteroatoms, selected from among oxygen, sulphur and nitrogen, while the ring may be connected to the molecule via a carbon atom or, if present, via a nitrogen atom. Unless otherwise stated, a spirocyclic ring may be provided with a keto group. Examples include:

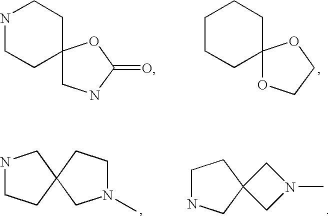

By the term “optionally substituted” is meant, unless stated otherwise, within the scope of the invention the above-mentioned group, optionally substituted by a lower-molecular group. Examples of lower-molecular groups regarded as chemically meaningful are groups consisting of 1-200 atoms. Preferably such groups have no negative effect on the pharmacological efficacy of the compounds.

For example the groups may comprise:
Straight-chain or branched carbon chains, optionally interrupted by heteroatoms, optionally substituted by rings, heteroatoms or other common functional groups.
Aromatic or non-aromatic ring systems consisting of carbon atoms and optionally heteroatoms, which may in turn be substituted by functional groups.
A number of aromatic or non-aromatic ring systems consisting of carbon atoms and optionally heteroatoms which may be linked by one or more carbon chains, optionally interrupted by heteroatoms, optionally substituted by heteroatoms or other common functional groups.

The term halogen generally denotes fluorine, chlorine, bromine or iodine.

The compounds according to the invention may occur in the form of the individual optical isomers, mixtures of the individual enantiomers, diastereomers or racemates, in the form of the tautomers as well as in the form of the free bases or the corresponding acid addition salts with pharmacologically acceptable acids—such as for example acid addition salts with hydrohalic acids, for example hydrochloric or hydrobromic acid, or organic acids, such as for example oxalic, fumaric, diglycolic or methanesulphonic acid.

Where a hyphen open on one side “-” is used in the structural formula of a substituent, this hyphen is to be understood as the linkage point to the remainder of the molecule. The substituent replaces the corresponding groups R², R⁶, etc. If no hyphen open on one side is used in the structural formula of a substituent, the linkage point to the remainder of the molecule is clear from the structural formula itself.

The substituent R¹ may denote a group selected from among hydrogen, CO—CH₃, CO—CH₂—R⁴, CO—CHMe-R⁴, CO—OR⁴, CO—SR⁴, CO—NH₂ and CO—NHR⁴ preferably CO—CH₃ and CO—CH₂—R⁴. Particularly preferably the substituent R¹ denotes CO—CH₃.

The substituent R² may denote a group selected from among C₃₋₆-cycloalkyl, C₁₋₄-alkyl-C₃₋₆-cycloalkyl, C₂₋₄-alkenyl-C₃₋₆-cycloalkyl, C₂₋₄-alkynyl-C₃₋₆-cycloalkyl, C₃₋₆-cycloalkenyl, C₁₋₆-alkyl-C₃₋₆-cycloalkenyl, C₂₋₄-alkenyl-C₅₋₆-cycloalkenyl, C₂₋₄-alkynyl-C₅₋₆-cycloalkenyl, C₅₋₆-cycloalkynyl, C₁₋₆-alkyl-C₅₋₆-cycloalkynyl, C₂₋₄-alkenyl-C₅₋₆-cycloalkynyl and C₂₋₄-alkynyl-C₅₋₆-cycloalkynyl-; preferably C₃₋₆-cycloalkyl, C₁₋₆-alkyl-C₃₋₆-cycloalkyl- and C₂₋₄-alkenyl-C₃₋₆-cycloalkyl-; particularly preferably C₃₋₆-cycloalkyl, particularly preferably cyclopropyl, which may optionally be substituted by one or two of the groups CH₃, F, OCH₃, OH or NH₂.

The substituent R³ may represent a group selected from among C₆-C₁₄-aryl, C₁₋₆-alkyl-C₆-C₁₄-aryl, C₂₋₆-alkenyl-C₆-C₁₄-aryl, C₂₋₆-alkynyl-C₆-C₁₄-aryl, C₅-C₁₀-heteroaryl, C₁₋₁₂-alkyl-C₅-C₁₀-heteroaryl, C₃₋₁₂-alkenyl-C₅-C₁₀-heteroaryl, C₃₋₁₂-alkynyl-C₅-C₁₀-heteroaryl, C₃₋₆-cycloalkyl, C₁₋₆-alkyl-C₃₋₆-cycloalkyl, C₂₋₄-alkenyl-C₃₋₆-cycloalkyl, C₂₋₄-alkynyl-C₃₋₆-cycloalkyl, C₅₋₆-cycloalkenyl, C₁₋₆-alkyl-C₅₋₆-cycloalkenyl, C₂₋₄-alkenyl-C₅₋₆-cycloalkenyl, C₂₋₄-alkynyl-C₅₋₆-cycloalkenyl, C₅₋₆-cycloalkynyl, C₁₋₆-alkyl-C₅₋₆-cycloalkynyl, C₂₋₄-alkenyl-C₅₋₆-cycloalkynyl- and C₂₋₄-alkynyl-C₅₋₆-cycloalkynyl, preferably C₆-C₁₄-aryl and C₃₋₆-cycloalkyl, preferably phenyl and C₅₋₆-cycloalkyl, particularly preferably phenyl, which may optionally be substituted by a group R⁵ and up to three groups R⁶.

R³ may preferably represent optionally substituted 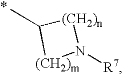
wherein n, m, independently of one another denote 1 or 2.

The substituent R⁴ may represent an optionally substituted group selected from among C₁₋₄-alkyl, C₂₋₁₀-alkenyl, C₂₋₁₀-alkynyl, C₃₋₆-cycloalkyl-C₁₋₄-alkyl, C₃₋₆-cycloalkyl-C₃₋₁₀-alkenyl, C₃₋₆-cycloalkyl-C₃₋₁₀-alkynyl, C₆-C₁₄-aryl, C₆-C₁₄-aryl-C₁₋₄-alkyl, C₅-C₁₀-heteroaryl, C₅-C₁₀-heteroaryl-C₁₋₄-alkyl- and haloalkyl, preferably C₁₋₃-alkyl, C₆-C₁₄-aryl-C₁₋₄-alkyl- and haloalkyl, particularly preferably methyl, ethyl, n-propyl, i-propyl, c-propyl, phenyl, —CH₂-cpropyl, —CH₂-phenyl and CF₃.

The substituent R⁵ may represent a group selected from among CONR⁸R⁹, NR⁸COR⁹, NR⁸R⁹, OR⁹ and —C₁₋₄-alkyl-CONR⁸R⁹; preferably CONR⁸R⁹, NR⁸COR⁹, NR⁸R⁹, OR⁹ and —CH₂—CONR⁸R⁹.

The substituent R⁶, which may be identical or different, may denote a group selected from among F, Cl, Br, OH, CN, CF₃, CHF₂ or an optionally substituted group, selected from among O—C₁₋₃-alkyl, O—C₃₋₄-alkenyl, O—C₃₋₄-alkynyl, C₁₋₃-alkyl, C₂₋₆-alkenyl and C₂₋₃-alkynyl, C₃₋₆-cycloalkyl-C₁₋₄-alkyl, C₃₋₆-cycloalkyl-C₂₋₄-alkenyl, C₃₋₆-cycloalkyl-C₂₋₄-alkynyl, C₃₋₆-cycloalkenyl-C₁₋₄-alkyl, C₃₋₆-cycloalkenyl-C₃₋₁₀-alkenyl, C₃₋₆-cycloalkenyl-C₂₋₄-alkynyl, C₆-C₁₄-aryl-C₁₋₄-alkyl, C₆-C₁₄-aryl-C₂₋₄-alkenyl, C₆-C₁₄-aryl-C₂₋₄-alkynyl, C₅-C₁₀-heteroaryl-C₁₋₄-alkyl, C₅-C₁₀-heteroaryl-C₂₋₄-alkenyl- and C₅-C₁₀-heteroaryl-C₂₋₄-alkynyl,
preferably F, Cl, Br, C₁₋₃-alkyl, OH, CN, —O—C₁₋₃-alkyl, C₂₋₃-alkenyl, C₂₋₃-alkynyl, CF₃ and CHF₂, particularly preferably F, Cl, Br and CF₃ or an optionally substituted group, selected from among O—C₁₋₃-alkyl, C₁₋₃-alkyl, C₃₋₆-cycloalkyl-C₁₋₄-alkyl- and C₆-C₁₄-aryl-C₁₋₄-alkyl, particularly preferably F, Cl, CF₃, or an optionally substituted group O—C₁₋₃-alkyl or C₁₋₃-alkyl.

The substituent R⁷ may represent a group selected from among hydrogen, COR⁹ and CONR⁸R⁹ or
a group, selected from among C₁₋₁₀-alkyl, C₃₋₁₀-alkenyl, C₃₋₁₀-alkynyl, C₃₋₆-cycloalkyl-C₁₋₄-alkyl, C₃₋₆-cycloalkyl-C₃₋₁₀-alkenyl, C₃₋₆-cycloalkyl-C₃₋₁₀-alkynyl, C₃₋₆-cycloalkenyl-C₁₋₄-alkyl, C₃₋₆-cycloalkenyl-C₃₋₁₀-alkenyl, C₃₋₆-cycloalkenyl- C₃₋₁₀-alkynyl, C₆-C₁₄-aryl, C₁₋₁₀-alkyl-C₆-C₁₄-aryl, C₂₋₁₀-alkenyl-C₆-C₁₄-aryl, C₂₋₁₀-alkynyl-C₆-C₁₄-aryl, C₅-C₁₀-heteroaryl, C₁₋₁₂-alkyl-C₅-C₁₀-heteroaryl, C₃₋₁₂-alkenyl-C₅-C₁₀-heteroaryl- and C₃₋₁₂-alkynyl-C₅-C₁₀-heteroaryl, which may optionally be substituted by a group R¹⁴ and by a group R¹³. Preferably R⁷, which may be identical or different may denote hydrogen, COR⁹ or CONR⁸R⁹, particularly preferably COR⁹ or CONR⁸R⁹.

The substituent R⁸ may denote hydrogen or
an optionally substituted group, selected from among C₁₋₁₀-alkyl, C₃₋₁₀-alkenyl, C₃₋₁₀-alkynyl, C₃₋₆-cycloalkyl-C₁₋₄-alkyl, C₃₋₆-cycloalkyl-C₃₋₁₀-alkenyl, C₃₋₆-cycloalkyl-C₃₋₁₀-alkynyl, C₃₋₆-cycloalkenyl-C₁₋₄-alkyl, C₃₋₆-cycloalkenyl-C₃₋₁₀-alkenyl, C₃₋₆-cycloalkenyl-C₃₋₁₀-alkynyl, C₆-C₁₄-aryl-C₁₋₄-alkyl, C₆-C₁₄-aryl-C₃₋₁₀-alkenyl- and C₆-C₁₄-aryl-C₃₋₁₀-alkynyl, C₅-C₁₀-heteroaryl, C₅-C₁₀-heteroaryl-C₁₋₄-alkyl, C₅-C₁₀-heteroaryl-C₁₋₄-alkenyl, C₅-C₁₀-heteroaryl-C₁₋₄-alkynyl, C₁₋₄-alkyl-O—C₂₋₄-alkyl, C₁₋₄-alkyl-O—C₄₋₆-alkenyl- and C₁₋₄-alkyl-O—C₄₋₆-alkynyl. Preferably R⁸ may denote hydrogen or
an optionally substituted group, selected from among C₁₋₁₀-alkyl, C₃₋₁₀-alkenyl, C₃₋₁₀-alkynyl and C₁₋₄-alkyl-O—C₂₋₄-alkyl, particularly preferably hydrogen or C₁₋₁₀-alkyl.

The substituent R⁹ may represent a group selected from among hydrogen or an optionally substituted group selected from among C₁₋₁₂-alkyl, C₃₋₁₂-alkenyl, C₃₋₁₂-alkynyl, C₃₋₆-cycloalkyl-C₁₋₁₂-alkyl, C₃₋₆-cycloalkyl-C₃₋₁₂-alkenyl, C₃₋₆-cycloalkyl-C₃₋₁₂-alkynyl, C₃₋₆-cycloalkenyl-C₁₋₄-alkyl, C₃₋₆-cycloalkenyl-C₃₋₁₀-alkenyl, C₃₋₆-cycloalkenyl-C₃₋₁₀-alkynyl, C₆-C₁₄-aryl-C₁₋₁₂-alkyl, C₆-C₁₄-aryl-C₃₋₁₂-alkenyl, C₆-C₁₄-aryl-C₃₋₁₂-alkynyl, C₆-C₁₄-aryl, C₁₋₁₂-alkyl-C₆-C₁₄-aryl, C₂₋₁₂-alkenyl-C₆-C₁₄-aryl, C₂₋₁₂-alkynyl-C₆-C₁₄-aryl, C₅-C₁₀-heteroaryl, C₅-C₁₀-heteroaryl-C₁₋₂-alkyl, C₅-C₁₀-heteroaryl-C₃₋₁₂-alkenyl, C₅-C₁₀-heteroaryl-C₃₋₁₂-alkynyl, C₃₋₈-cycloalkyl, C₃₋₈-cycloalkenyl, NR¹¹R¹²—C₃₋₈-cycloalkyl, NR¹¹R¹²—C₄₋₈-cycloalkenyl- and NR¹¹R¹²—C₅₋₈-cycloalkynyl or a optionally substituted C₃₋₈-heterocycloalkyl-(CH₂)_{q} group, containing at least one NR¹⁰ group in the 3- to 8-membered heterocyclic group.

Preferably R⁹ may represent hydrogen or
an optionally substituted group, selected from among C₁₋₁₂-alkyl, C₃₋₁₂-alkenyl, C₃₋₁₂-alkynyl, C₃₋₆-cycloalkyl-C₁₋₁₂-alkyl, C₆-C₁₄-aryl, C₁₋₁₂-alkyl-C₆-C₁₄-aryl, C₂₋₁₂-alkenyl-C₆-C₁₄-aryl, C₂₋₁₂-alkynyl-C₆-C₁₄-aryl, C₅-C₁₀-heteroaryl, C₅-C₁₀-heteroaryl-C₁₋₁₂-alkyl, C₅-C₁₀-heteroaryl-C₃₋₁₂-alkenyl, C₅-C₁₀-heteroaryl-C₃₋₁₂-alkynyl, C₃₋₈-cycloalkyl, C₃₋₈-cycloalkenyl and NR¹¹R¹²—C₃₋₈-cycloalkyl, or
an optionally substituted C₃₋₈-heterocycloalkyl-(CH₂)_{q}— containing at least one NR¹⁰ group in the 3- to 8-membered heterocyclic group.

Particularly preferably R⁹ may represent hydrogen or an optionally substituted group, selected from among C₁₋₁₂-alkyl, C₃₋₁₂-alkenyl, C₃₋₁₂-alkynyl, C₃₋₆-cycloalkyl-C₁₋₁₂-alkyl, C₆-C₁₄-aryl, C₁₋₁₂-alkyl-C₆-C₁₄-aryl, C₂₋₁₂-alkenyl-C₆-C₁₄-aryl, C₂₋₁₂-alkynyl-C₆-C₁₄-aryl, C₅-C₁₀-heteroaryl, C₅-C₁₀-heteroaryl-C₁₋₁₂-alkyl, C₅-C₁₀-heteroaryl-C₃₋₁₂-alkenyl, C₅-C₁₀-heteroaryl-C₃₋₁₂-alkynyl, C₃₋₈-cycloalkyl, C₃₋₈-cycloalkenyl and NR¹¹R¹²—C₃₋₈-cycloalkyl, particularly preferably C₁₋₁₂-alkyl, C₃₋₁₂-alkenyl, C₃₋₁₂-alkynyl, C₃₋₆-cycloalkyl-C₁₋₁₂-alkyl, C₆-C₁₄-aryl, C₁₋₁₂-alkyl-C₆-C₁₄-aryl, C₂₋₁₂-alkenyl-C₆-C₁₄-aryl, C₂₋₁₂-alkynyl-C₆-C₁₄-aryl, C₃₋₈-cycloalkyl, C₃₋₈-cycloalkenyl and NR¹¹R¹²—C₃₋₈-cycloalkyl, or
an optionally substituted group selected from among the general formulae (A1) to (A12) 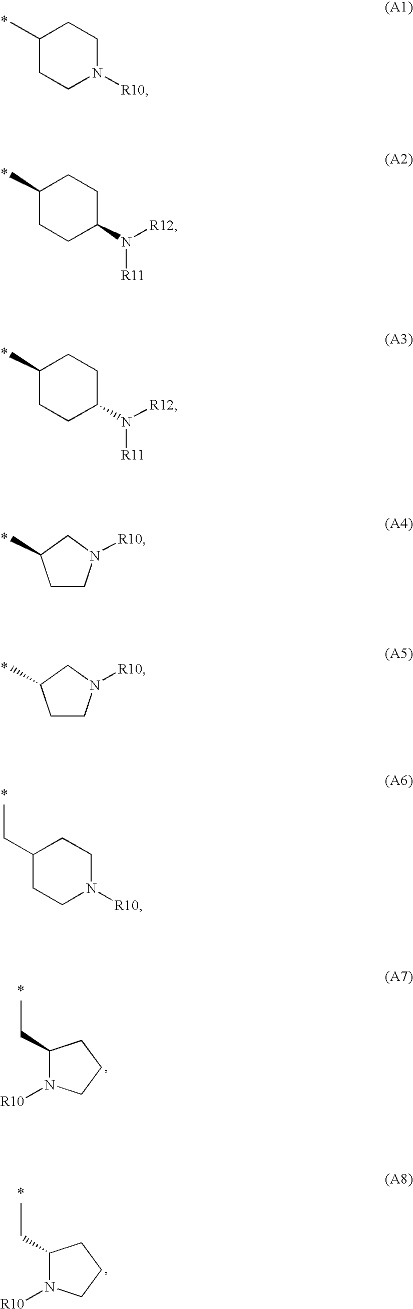 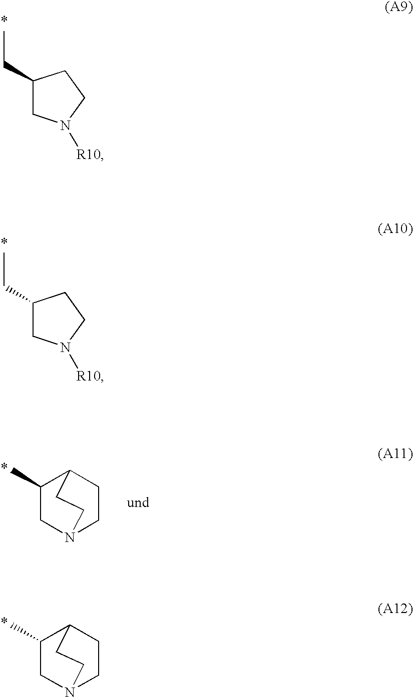
The substituents R⁸ and R⁹ may together form a saturated or unsaturated 4- to 7-membered alkyl bridge which optionally contains an O atom or an S(O)ₚ group,
   wherein p, q independently of one another denote 0, 1 or 2, or
NR⁸R⁹ denotes a 5- to 6-membered heterocyclic group, optionally containing a further N atom and optionally substituted by a group selected from among R¹⁰, NR¹¹R¹² and NR¹¹R¹²C₁₋₄-alkyl, or
   a group 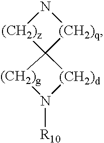
   wherein
   z, q, g, d independently of one another denote 1 , 2 or 3.
Preferably R⁸ and R⁹ together form a saturated or unsaturated 4- to 7-membered alkyl bridge which optionally contains an O atom or an S(O)ₚ group,
   wherein p, q independently of one another denote 0, 1 or 2; or
NR⁸R⁹ denotes an optionally substituted group selected from among the general formulae (B1) to (B8) 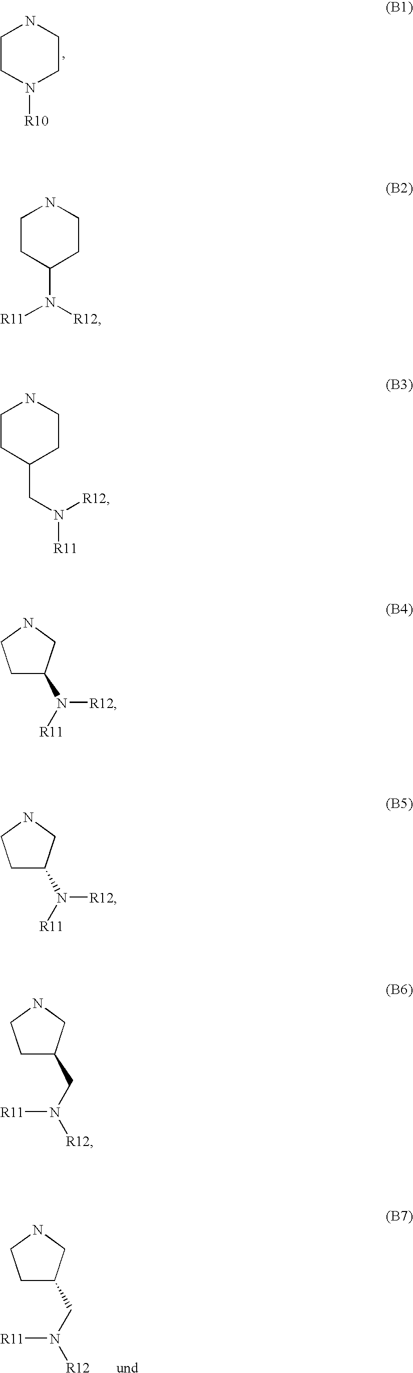 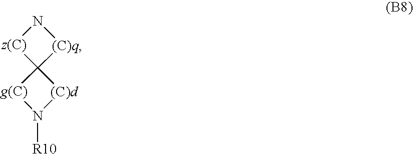
   wherein
   z, q, g, d independently of one another denote 1, 2 or 3.

The substituent R¹⁰ may denote a group selected from among
hydrogen or
an optionally substituted group, selected from among C₁₋₁₀-alkyl, C₃₋₁₀-alkenyl, C₃₋₁₀-alkynyl, C₃₋₇-cycloalkyl-C₁₋₁₀-alkyl, C₃₋₇-cycloalkyl-C₃₋₁₀-alkenyl, C₃₋₇-cycloalkyl-C₃₋₁₀-alkynyl, C₃₋₇-cycloalkyl, C₁₋₆-alkyl-C₃₋₇-cycloalkyl, C₂₋₄-alkenyl-C₃₋₇-cycloalkyl, C₂₋₄-alkynyl-C₃₋₇-cycloalkyl, tetrahydropyranyl and (NR⁴)₂CH—C₁₋₁₀-alkyl.
Preferably R¹⁰ may denote hydrogen or
   an optionally substituted group, selected from among C₁₋₁₀-alkyl, C₃₋₇-cycloalkyl-C₁₋₁₀-alkyl, C₃₋₇-cycloalkyl, C₁₋₆-alkyl-C₃₋₇-cycloalkyl, tetrahydropyranyl and (NR⁴)₂CH—C₁₋₁₀-alkyl.

The substituents R¹¹, R¹², which may be identical or different, may represent hydrogen or
an optionally substituted group, selected from among C₁₋₁₀-alkyl, C₃₋₁₀-alkenyl and C₃₋₁₀-alkynyl, C₃₋₆-cycloalkyl-C₁₋₄-alkyl- and C₃₋₆-cycloalkyl, preferably from C₁₋₁₀-alkyl, C₃₋₁₀-alkenyl, C₅₋₆-cycloalkyl-C₁₋₄-alkyl- and C₅₋₆-cycloalkyl, or
R¹¹ and R¹² together form a 4- to 7-membered, preferably 5- to 6-membered alkyl chain which optionally contains a heteroatom.

The substituent R¹³ may represent F, Cl, Br, OH, CN, CF₃, CHF₂ or C₁₋₄-alkyl-O—.

The substituent R¹⁴ may represent NR¹¹R¹² or an optionally substituted C₃₋₈-heterocycloalkyl-(CH₂)_{q} group, containing at least one NR¹⁰ group in the 3- to 8-membered heterocyclic group,
preferably an optionally substituted group selected from among the general formulae (A1) to (A12)

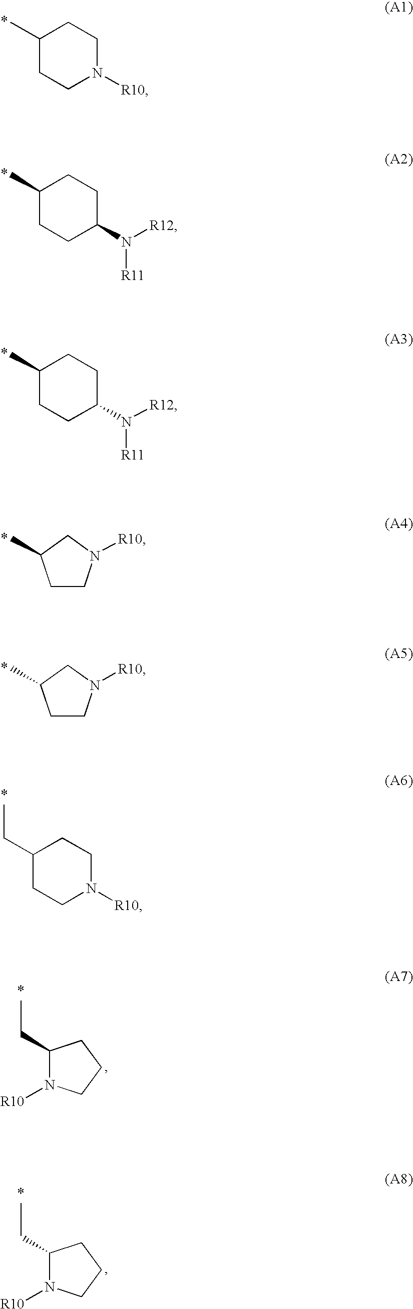

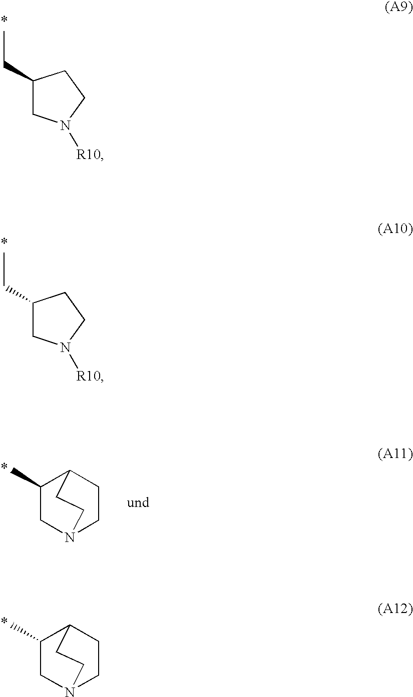

The substituents R¹³ and R¹⁴ may together form a saturated or unsaturated 4- to 7-membered alkyl bridge, preferably a 5 to 6 membered alkyl bridge which optionally contains an O atom or an S(O)ₚ group, wherein p denotes 0, 1 or 2; preferably 0 or 2.

Preparation Processes

The compounds of general formula (I) may be prepared according to the following synthesis scheme (Diagram 1-4), wherein the substituents of general formula (I) have the above-mentioned meanings. These processes are intended as an illustration of the invention without restricting it to their content.

Diagram 1:

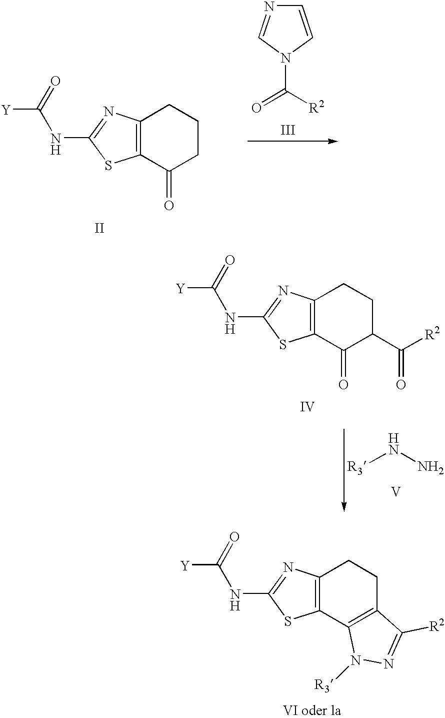

The group R² may have the meanings given above.

R₃′ may represent an optionally substituted group selected from among 4-PhCOOMe, 4-PhNO₂ and 4-piperidyl, cis/trans-4-alkoxycarbonylcylohexyl and 4-methoxycarbonyl-methy-phenyl.

Y may represent C₁-C₄-alkyl or —S—C₁-C₄-alkyl, preferably methyl or ethyl.

According to Diagram 1 a compound of formula II is reacted with a compound of formula III to obtain a compound of formula IV. Then the compound of formula IV is reacted with a compound of formula V and cyclised to form a compound of formula VI or Ia.
Diagram 2a:

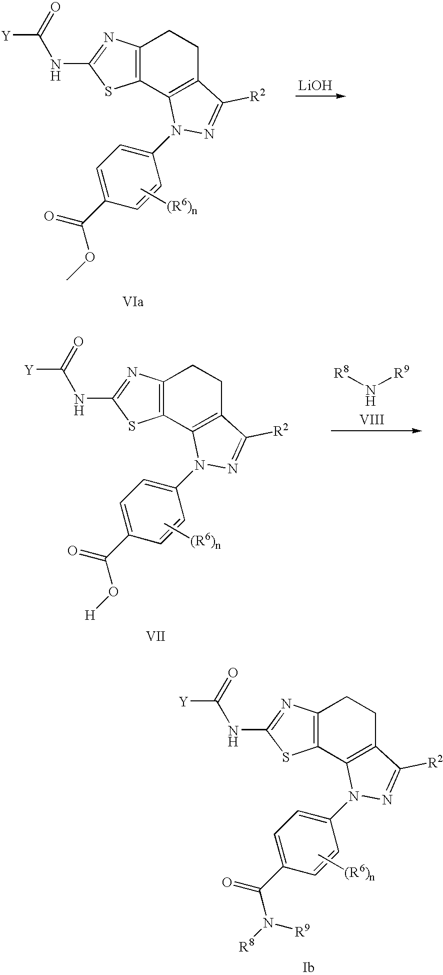

The groups R², R⁶, R⁸ and R⁹ may have the meanings given above.

According to Diagram 2a a compound of formula VIa is reacted with an alkali metal hydroxide, preferably LiOH, to obtain a compound of formula VII. Then the compound of formula VII is reacted with a compound of formula VIII to obtain a compound of formula Ib.
Diagram 2b: 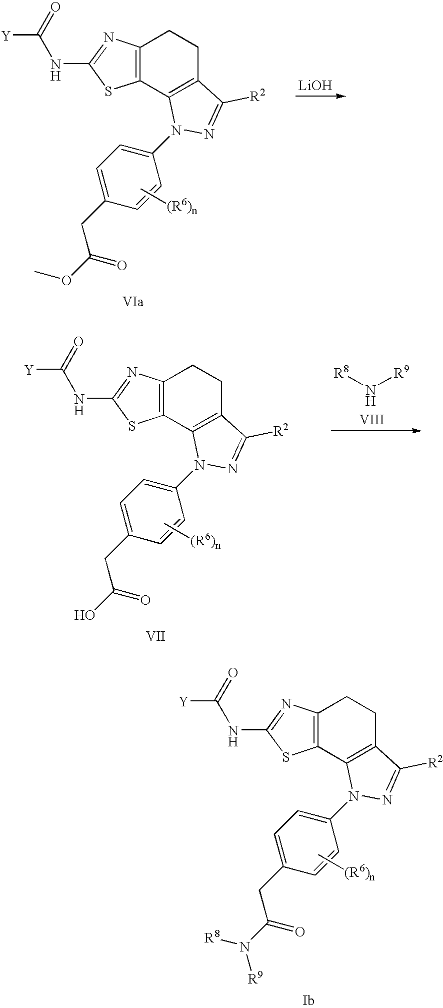
Diagram 2c: 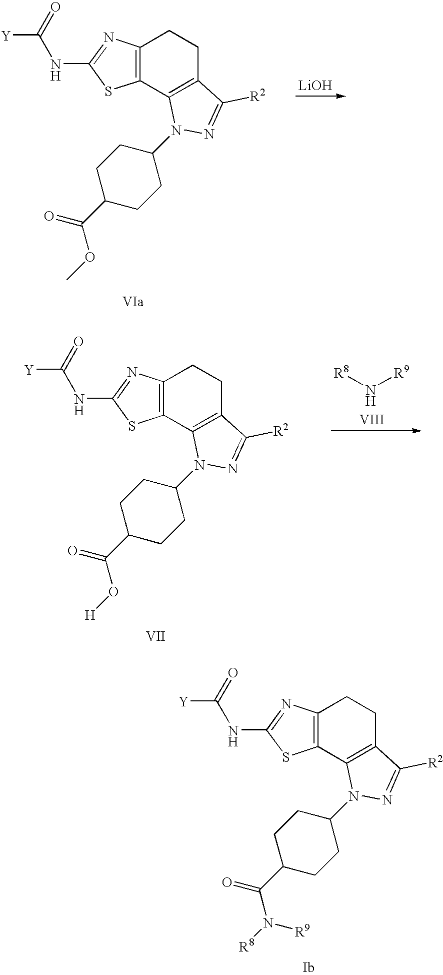
Diagram 3:

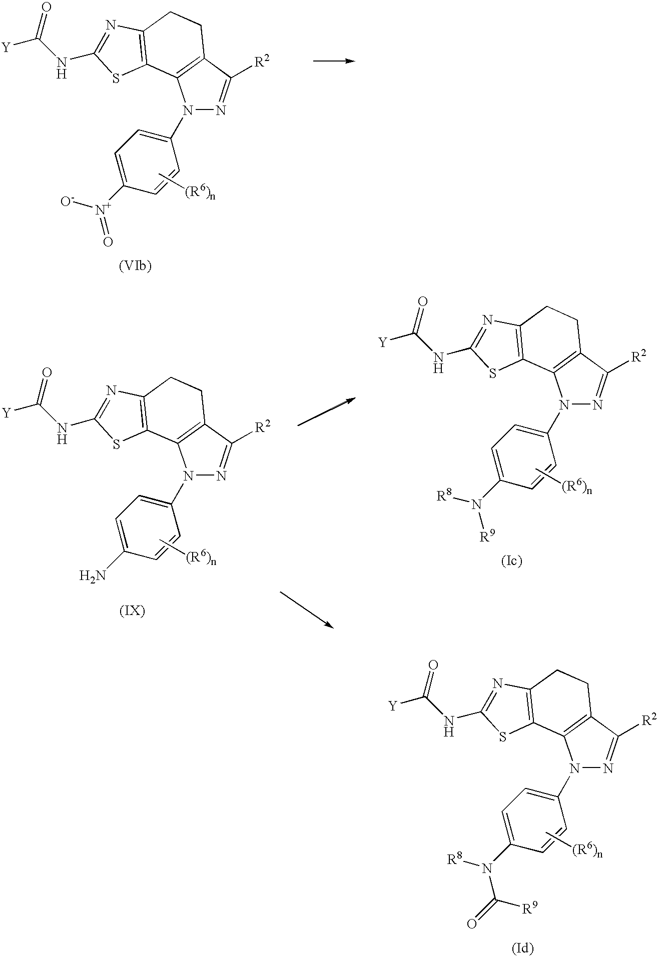

The groups R², R⁶, R⁸ and R⁹ may have the meanings given above.

According to Diagram 3 a compound of formula VIb is reduced with H₂/PdC at the nitro group to obtain a compound of formula IX. Then the compound of formula IX is reacted with a compound of formula VIII to obtain a compound of formula Ic or Id.
Diagram 4:

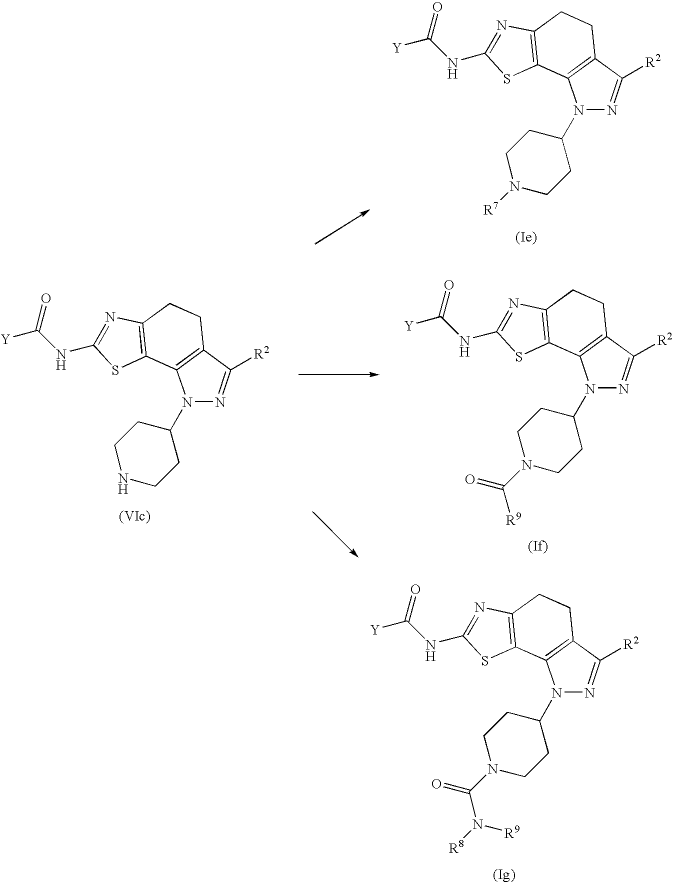

The groups R², R⁶, R⁸ and R⁹ may have the meanings given above.

According to Diagram 4 a compound of formula VIc is reacted to obtain a compound of formula Ie, If or Ig.

The new compounds of general formula (I) may be prepared analogously to the following Examples. The Examples described below are intended as an illustration of the invention without restricting it.

Synthesis of the Reagents

1) Compounds of Formula III

1.1) Imidazol-1-yl-cyclopropyl-methanone (III.1)

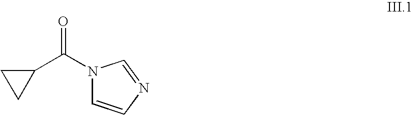

75 g (0.46 mol) CDI and 30.0 g (0.35 mol) cyclopropanecarboxylic acid are stirred for 20 h at RT. Then the reaction mixture is washed twice with 200 mL Kochsalzlpösung, the organic phase is dried and the solvent is eliminated i. vac. Yield: 45.5 g (96%).
1.2) cyclopentyl-imidazol-1-yl-methanone (III.2)

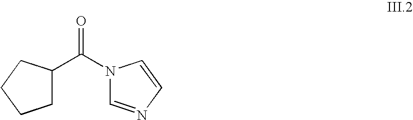

17.70 g (155.07 mmol) cyclopentanecarboxylic acid are placed in 350 mL dichloromethane, 30.00 g (181.00 mmol) CDI are added batchwise. The reaction mixture is stirred for 3 hours at ambient temperature, then cooled to 0° C. and some ice is added. The mixture is stirred for 0.1 hours, then extracted with semisaturated sodium chloride solution. The organic phase is dried and evaporated to dryness. Yield: 25.00 g (98%).

The following compounds are prepared analogously:
1.3) imidazol-1-yl-(1-methyl-cyclopropyl)-methanone (III.3)

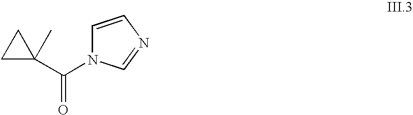

10.50 g (0.105 mol) 1-methylcyclopropanecarboxylic acid and 22.00 g (0.136 mol) CDI are used. Yield: 16.10 g (94%)
1.4) cyclobutyl-imidazol-1-yl-methanone (III.4)

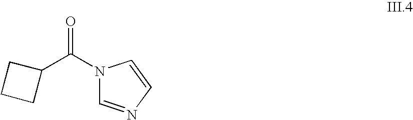

20.00 g (200 mmol) cyclobutanecarboxylic acid and 37.00 g (224 mmol) CDI are used. Yield: 29.10 g (97%)

2) Compounds of Formula V

2.1) methyl 3-chloro-4-hydrazino-benzoate (V.1)

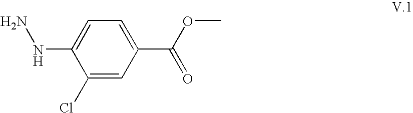

31.99 g (0.172 mol) methyl-4-amino-3-chlorobenzoate are suspended in 160 mL conc. hydrochloric acid and cooled to −10° C. A solution of 11.98 g (0.174 mol) sodium nitrite and 160 mL water is added dropwise at −5° C. 170.98 g (0.759 mol) tin-(II)-chloride in 140 mL hydrochloric acid are added dropwise to the resulting solution. A thick precipitate is formed. The reaction mixture is frozen overnight. After thawing the suspension is made basic with 10 molar sodium hydroxide solution. After the addition of dichloromethane the product dissolves and is separated off with the organic phase. The latter is washed with water, dried and evaporated to dryness. The residue is purified by chromatography. Yield: 18.3 g (53%).

The following compounds are prepared analogously:
2.2) methyl (3-chloro-4-hydrazino-phenyl)-acetate (V.2)

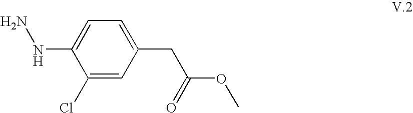

5.00 g (25 mmol) methyl (4-amino-3-chloro-phenyl)-acetate, 80 mL conc. hydrochloric acid, 1.90 g (28 mmol) sodium nitrite and 22.60 g (100 mmol) tin-(II)-chloride-dihydrate in 30 mL hydrochloric acid are used. Yield: 2.33 g (43%).
2.3) methyl 2-chloro-4-hydrazino-benzoate (V.3)

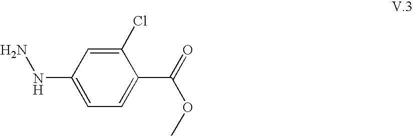

49.08 g (0.221 mol) 4-amino-2-chloro-methyl benzoate hydrochloride, 250 mL conc. hydrochloric acid, 18.23 g (0.264 mol) sodium nitrite and 199.12 g (0.883 mol) tin(II)-chloride-dihydrate in 250 mL conc. hydrochloric acid are used. Yield after crystallisation from isopropanol: 24.7 g (56%).
2.4) methyl 4-hydrazino-2-methoxy-benzoate (V.4)

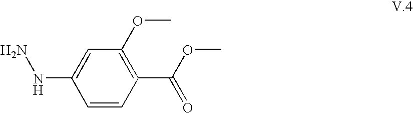

25.00 g (0.138 mol) methyl-4-amino-2-methoxybenzoate are suspended in 124 mL conc. hydrochloric acid and cooled to 2° C. A solution of 11.42 g (0.166 mol) sodium nitrite in 124 mL water is slowly added dropwise, then the mixture is stirred for 1 hour while cooling with an ice bath. A solution of 60.45 g (0.318 mol) sodium pyrosulphite in 248 mL water (adjusted to pH 6.5 with sodium hydroxide) is taken and the cooled diazonium solution is slowly added dropwise. The pH is maintained between 6.3 and 6.5. The reaction mixture is refluxed for 4 hours with stirring, then added to 260 mL conc. hydrochloric acid and left to stand for 16 hours at ambient temperature. Then the solution is made basic and extracted with tetrahydrofuran and ethylacetate. The organic phase is dried and evaporated to dryness. The residue is crystallised from isopropanol, then the hydrochloride is precipitated. Yield: 9.72 g (30%).
2.5) 2-chloro-4-nitro-phenyl-hydrazine hydrochloride (V.5)

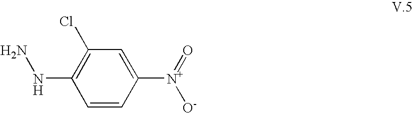

25.00 g (0.140 mol) 2-chloro-4-nitro-fluorobenzene and 7.00 g (0.140 mol) hydrazine hydrate are placed in 45 mL 1-methyl-2-pyrrolidone and the mixture is stirred for 3.5 hours at 65° C. After cooling the reaction mixture is mixed with water, the precipitate formed is suction filtered. The crystals damp with water are recrystallised from isopropanol, then precipitated as the hydrochloride. Yield: 11.4 g (36%).

2.6)

2.6.1) tert-butyl 4-(tert-butoxycarbonyl-hydrazono)-piperidine-1-carboxylate

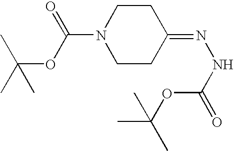

10.00 g (50.19 mmol) BOC-piperidone and 6.63 g (50.19 mmol) BOC-hydrazine are refluxed with 20 g molecular sieve in 250 mL n-hexane for 4 hours with stirring. Then the mixture is evaporated down, the residue is stirred for 2 hours in acetonitrile, suction filtered through kieselguhr and evaporated down. Yield: 8.00 g (51%).
2.6.2) piperidin-4-yl-hydrazine (V.6)

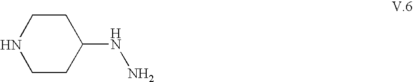

8.00 g (25.53 mmol) tert-butyl 4-(tert-butoxycarbonyl-hydrazono)-piperidine-1-carboxylate are stirred in 26.00 mL (26 mmol) borane-tetrahydrofuran complex (1 molar) for 24 hours at ambient temperature, then combined with 4molar hydrochloric acid in dioxane, stirred for 24 hours at ambient temperature. The reaction mixture is concentrated in vacuo, crystallised and suction filtered. The crude product is mixed with water, saturated with sodium chloride and extracted with tetrahydrofuran. The aqueous phase is evaporated down, the residue is triturated with tetrahydrofuran, filtered and evaporated down. The hydrochloride is precipitated. Yield: 4.30 g (90%).

2.7)

2.7.1) ethyl cis/trans-4-(N′-tert-butoxycarbonyl-hydrazino)-cyclohexanecarboxylate

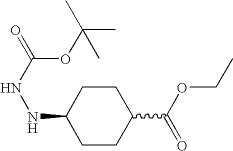

10.50 g (61.69 mmol) ethyl 4-oxo-cyclohexanylcarboxylate are placed in 200 mL hexane, 8.15 g (61.69 mmol) tert.-butylcarbazate are added. The mixture is refluxed for 4 hours with stirring, cooled to ambient temperature and combined with 70 mL (70 mmol) borane-tetrahydrofuran complex (1 molar). The reaction mixture is stirred for 16 hours at ambient temperature. Then 5 mL water are added and the mixture is evaporated down. The residue is combined with ethyl acetate and magnesium sulphate is added. The suspension is suction filtered, the filtrate is evaporated to dryness. The residue is separated by chromatography on a 2.51 silica gel column (cyclohexane/ethyl acetate). Yield: 6.97 g (40%) cis-compound and 7.32 g (42%) trans-compound
2.7.2) ethyl cis-4-hydrazino-cyclohexanecarboxylate (V.7)

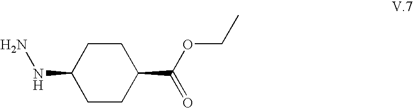

6.90 g (24.10 mmol) ethyl cis-4-(N′-tert-butoxycarbonyl-hydrazino)-cyclohexanecarboxylate are dissolved in 75 mL dioxane, 50 mL hydrochloric acid solution in dioxane (4 molar) is added. The reaction mixture is stirred for 16 hours at 40° C. After cooling diethyl ether is added, the precipitate is suction filtered, washed with diethyl ether and dried. Yield: 5.06 g (94%).
2.8) ethyl trans-4-hydrazino-cyclohexanecarboxylate (V.8)

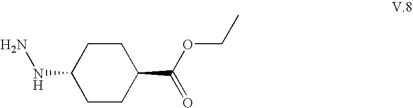

7.30 g (25.49 mmol) ethyl trans-4-(N′-tert-butoxycarbonyl-hydrazino)-cyclohexanecarboxylate are used. Yield: 5.60 g (99%).

3) Compounds of Formula VIII

3.1) tert-butyl cis-(4-pyrrolidin-1-yl-cyclohexyl)-carbamate

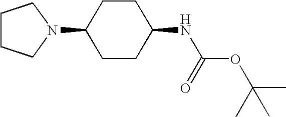

10.00 g (0.0467 mol) tert.butyl-cis-4-aminocyclohexanecarbamate, 12.10 g (0.0560 mol) 1,4-dibromobutane and 25.00 g (0.250 mol) potassium hydrogen carbonate are placed in 400 mL dimethylformamide, then stirred for 24 hours at ambient temperature. Then the mixture is evaporated down, the residue is extracted with diethyl ether and water. The organic phase is dried and evaporated to dryness. The still contaminated product is precipitated as a salt, crystallised from acetonitrile and liberated again. Yield: 6.0 g (48%).

The following compound is prepared analogously:

3.2)

3.2.1) tert-butyl cis-(4-piperidin-1-yl-cyclohexyl)-carbamate

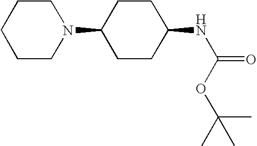

10.00 g (47 mmol) tert.-butyl-cis-4-aminocyclohexanecarbamate, 7.63 mL (56 mmol) 1,5-dibromopentane and 23.36 g (233.31 mmol) potassium hydrogen carbonate in 450 mL dimethylformamide are used. Yield: 14.23 g (100%)

The following compound is prepared analogously:
3.2.2) cis-4-piperidin-1-yl-cyclohexylamine dihydrochloride

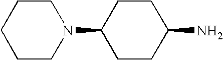

7.12 g (25 mmol) tert-butyl cis-4-piperidin-1-yl-cyclohexyl-carbamate and 201.54 mL (202 mmol) 1 molar ethereal hydrochloric acid are used. Yield: 8.44 g (100%).

3.3)

3.3.1) tert-butyl methyl-(cis-4-pyrrolidin-1-yl-cyclohexyl)-carbamate

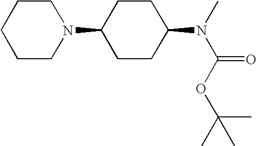

4.00 g (0.0149 mol) tert-butyl (cis-4-pyrrolidin-1-yl-cyclohexyl)-carbamate are placed in 40 mL dimethylformamide and 0.660 g (0.0165 mol) sodium hydride (60% in oil) are added. After foaming has ended 2.32 g (0.0163 mol) methyl iodide are added and the mixture is stirred at ambient temperature. The reaction mixture is washed with water and extracted with ethyl acetate, the organic phase is dried and evaporated to dryness. The residue is precipitated as the oxalate. Yield: 1.58 g (38%).
3.3.2) methyl-(cis-4-pyrrolidin-1-yl-cyclohexyl)-amine

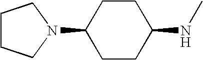

1.70 g (6 mmol) tert-butyl methyl-(cis-4-pyrrolidin-1-yl-cyclohexyl)-carbamate and 20 mL trifluoroacetic acid are placed in 100 mL dichloromethane, then stirred for 4 h at ambient temperature. Then the reaction mixture is evaporated down, the residue is precipitated as a salt. Yield: 1.45 g (94%).
3.4) tert-butyl cis-(4-cyclopropylmethyl-methylamino-cyclohex-1-yl)-carbamate

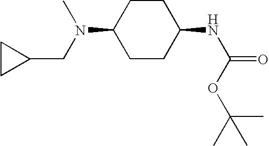

10 g (46.6 mmol) tert.butyl-cis-4-aminocyclohexanecarbamate and 3.5 mL (46.6 mmol) cyclopropylcarboxaldehyde are stirred in 500 mL dioxane for 3 h at ambient temperature. Then 20.8 g (93.3 mmol) sodium triacetoxyborohydride are added and stirring is continued overnight, 200 ml of 5% potassium carbonate solution are added and the mixture is stirred for 1 h. The phases are separated and the aqueous phase is extracted with methylene chloride. The organic phases are combined and extracted once with water, then dried and evaporated down. Crude product is applied to silica gel and separated on a silica gel column. The suitable fractions are combined, evaporated down and in 500 mL dichloroethane mixed with 3.8 mL 37% formalin solution and stirred for 3 h at ambient temperature. Then 10 g (48 mmol) sodium triacetoxyborohydride are added and the mixture is stirred overnight. The next day the mixture is extracted with 5% potassium carbonate solution, the phases are separated and the organic phase is extracted with saturated sodium chloride solution. The organic phase is dried and evaporated down. Yield: 6.49g (40.3%).

3.5)

3.5.1) ethyl-(cis-4-pyrrolidin-1-yl-cyclohexyl)-carbamate tert-butyl ester

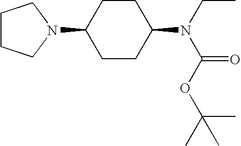

The base is liberated from 5.00 g (0.0139 mol) tert-butyl (cis-4-pyrrolidin-1-yl-cyclohexyl)-carbamate oxalate. 0.600 g (0.0150 mmol) sodium hydride (60% in oil) are placed in 15 mL dimethylacetamide and heated to 40° C. 25% of a solution of the free base in 15 mL dimethylacetamide are added dropwise. Then the mixture is heated to 55°-60° C. and the remaining solution is added dropwise. The reaction mixture is stirred for 1 hour at this temperature and for 1 hour at ambient temperature. After cooling to −10° C. 1.20 mL (0.0148 mol) ethyl iodide are added, then the mixture is stirred for 16 hours at ambient temperature. The reaction mixture is mixed with water and extracted with ethyl acetate. Combined organic phases are dried and evaporated to dryness. The residue is purified by chromatography. Yield: 0.170 g (4%).
3.5.2) ethyl-(cis-4-pyrrolidin-1-yl-cyclohexyl)-amine dichloride

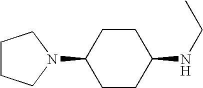

170 mg (0.573 mmol) tert-butyl ethyl-(cis-4-pyrrolidin-1-yl-cyclohexyl)-carbamate are dissolved in 5 mL methanolic hydrochloric acid (1.25 molar), and stirred for 16 hours at ambient temperature. Methanol is evaporated down in vacuo, the residue is combined with acetone. The precipitate formed is suction filtered, washed and dried. Yield: 100 mg (65%).

4.)

The following compounds may be prepared for the reaction of the compound of formula (VIc) to obtain the compound of formula (If):

4.1)

4.1.1) ethyl 1-cyclopentyl-piperidine-4-carboxylate

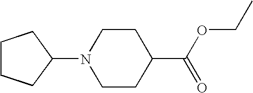

22.90 g (145.67 mmol) ethyl piperidine-4-carboxylate and 13.48 g cyclo-pentanone are placed in 400 mL tetrahydrofuran, 0.750 g p-toluenesulphonsaure and 12.50 mL (218.50 mmol) glacial acetic acid are added. The reaction mixture is stirred for 0.5 hours at ambient temperature, then 42.25 g (189.36 mmol) sodium acetoxyborohydride are added batchwise. The mixture is stirred for 16 h at ambient temperature, then evaporated down. The residue is extracted with dichloromethane and sodium carbonate solution. The organic phase is dried and evaporated to dryness. The aqueous phase is adjusted to pH 8 and extracted with chloroform. The organic phase is dried and evaporated to dryness. The two substances are combined. Yield: 39.70 g (100%)
4.1.2) 1-cyclopentyl-piperidine-4-carboxylic acid hydrochloride

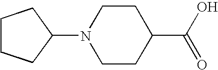

30.00 g (133.140 mmol) ethyl 1-cyclopentyl-piperidine-4-carboxylate and 150 mL conc. hydrochloric acid are placed in 150 mL water, then stirred for 16 hours at 100° C. The reaction mixture is concentrated in vacuo, during which time a precipitate is formed. This is suction filtered and dried. Yield: 12.1 g (39%)

4.2)

4.2.1) ethyl 1-propyl-piperidine-4-carboxylate

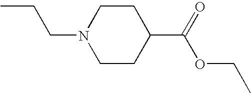

10.19 g (64.82 mmol) ethyl piperidine-4-carboxylate and 4.80 mL (66.45 mmol) propionaldehyde are placed in 150 mL ethanol, 6.55 mL (64.84 mmol) borane-pyridine complex are added. The reaction mixture is stirred for 4 h at ambient temperature, then evaporated down. The residue is extracted with dichloromethane and water, the organic phase is dried and evaporated to dryness. The residue is purified by chromatography. Yield: 1.90 g (15%).
4.2.2) 1-propyl-piperidine-4-carboxylic acid

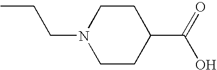

1.90 g (9.53 mmol) ethyl 1-propyl-piperidine-4-carboxylate and 30.00 mL (30 mmol) 1 molar sodium hydroxide solution are stirred in 10 mL methanol for 2 h at ambient temperature. Then the solution is adjusted to pH 6 with 1 molar hydrochloric acid and evaporated down. The residue is dissolved in methanol, filtered through silica gel. The filtrate is evaporated down, stirred with methanol. Yield: 1.70 g (100%).

The following compounds may be prepared for the reaction of the compound of formula (IX) to obtain the compound of formula (Id):
4.3) 1-cyclopentyl-piperidine-4-carbonyl chloride

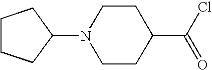

65 mg (0.278 mmol) 1-cyclopentyl-piperidine-4-carboxylic acid hydrochloride and 100 μL (1.38 mmol) thionyl chloride are placed in 8 mL toluene and 50 μL dimethylformamide, refluxed for 3 h with stirring. Then the mixture is evaporated down, combined with toluene and evaporated down again. Further reacted directly.

The following compound is prepared analogously:
4.4) 1-propyl-piperidine-4-carbonyl chloride

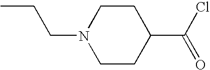

240 mg (1.40 mmol) 1-propyl-piperidine-4-carboxylic acid and 2 mL (27.57 mmol) thionyl chloride are used. Yield: 270 mg (85%)

Synthesis of the Intermediate Compounds

5) Compounds of Formula IV

5.1) N-(6-cyclopropylcarbonyl-7-oxo-4,5,6,7-tetrahydrobenzothiazol-2-yl)-acetamide (IV.1)

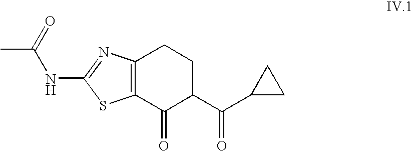

34.0 g (0.16 mol) N-(7-oxo-4,5,6,7-tetrahydrobenzothiazol-2-yl)-acetamide are placed in 3.5 L THF, cooled to −30° C. and 500 mL of a 1 molar solution of LHMDS are added dropwise at max. −20° C. After the addition has ended the mixture is stirred for 4 hours at −30° C. to −20° C. Then 45.0 g (0.33 mol) imidazol-1-yl-cyclopropyl-methanone dissolved in 50 mL THF are added dropwise at max. −20° C. The mixture is left overnight to come up to RT and then heat carrying liquid gas is piped in until pH 3 is reached. The yellow suspension formed is added to 1500 mL phosphate buffer, the org. phase is separated off and the aqueous phase is extracted once with ethyl acetate. The org. phases are dried on MgSO₄ and evaporated down i. vac. The oily residue crystallises overnight and after the addition of some acetonitrile the product is suction filtered and dried. Yield: 33.2 g (74%).
5.2) N-(6-cyclobutanecarbonyl-7-oxo-4,5,6,7-tetrahydro-benzothiazol-2-yl)-acetamide (IV.2)

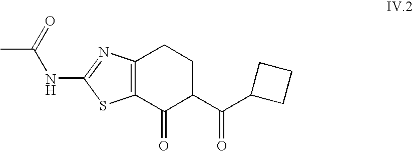

20.00 g (93.22 mmol) N-(7-oxo-4,5,6,7-tetrahydro-benzothiazol-2-yl)-acetamide are placed in 400 mL tetrahydrofuran and cooled to −70° C. 280mL (280mmol) lithium-bis-(trimethylsilyl)-amide (LHMDS) are slowly added, then stirred for 3 hours at −60° to −70° C. 18.00 g (120 mmol) cyclobutyl-imidazol-1-yl-methanone are added dropwise in 100 mL tetrahydrofuran, the reaction mixture is allowed to come up to ambient temperature within 16 hours. Then it is acidified while being cooled with a 4 molar hydrochloric acid solution in dioxane, phosphate buffer is added, the mixture is adjusted to pH 6.5 with sodium carbonate solution. After the addition of ethyl acetate and sodium chloride solution the mixture is extracted. The organic phase is dried and evaporated to dryness. 13.30 g (66%).

The following compounds are prepared analogously:
5.3) N-[6-(1-methyl-cyclopropanecarbonyl)-7-oxo-4,5,6,7-tetrahydro-benzothiazol-2-yl]-acetamide (IV.3)

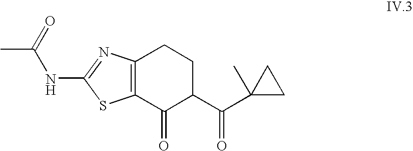

12.00 g (57.07 mmol) N-(7-oxo-4,5,6,7-tetrahydro-benzothiazol-2-yl)-acetamide, 172 mL (172 mmol) LHMDS and 16.10 g (98.63 mmol) imidazol-1-yl-(1-methyl-cyclopropyl)-methanone are used. Yield: 24.70 g (100%). HPLC: method B, RT=1.59 min, MH+=293
5.4) N-(6-cyclopentanecarbonyl-7-oxo-4,5,6,7-tetrahydro-benzothiazol-2-yl)-acetamide (IV.4)

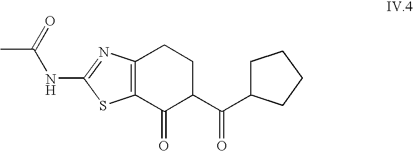

20.00 g (93.22 mmol) N-(7-oxo-4,5,6,7-tetrahydro-benzothiazol-2-yl)-acetamide, 280.00 mL (280.00 mmol) LHMDS and 25.00 g (152.25 mmol) cyclopentyl-imidazol-1-yl-methanone are used. Yield: 21.56 g (53%).

6) Compounds of Formula VIa

6.1) methyl 4-(7-acetylamino-3-cyclopropyl-4,5-dihydro-pyrazolo[3α,4′:3,4]benzo[1,2-d]thiazol-1-yl)-3-chloro-benzoate (VIa.1)

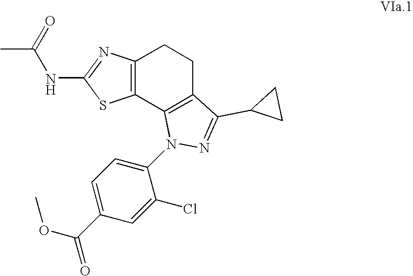

2.00 g (0.00719 mol) N-(6-cyclopropylcarbonyl-7-oxo-4,5,6,7-tetrahydrobenzothiazol-2-yl)-acetamide are placed in 50 mL glacial acetic acid, 1.75 g (0.00872 mol) methyl 3-chloro-4-hydrazino-benzoate are added. The reaction mixture is stirred for 90 h at ambient temperature. Then the glacial acetic acid is evaporated down in vacuo, the residue is extracted with 5% potassium carbonate solution and ethyl acetate. The combined organic phases are dried, evaporated to dryness and then crystallised from acetonitrile. The mixture of isomers is separated by chromatography. Yield: 1.61 g (51%).

The following compounds are prepared analogously:
6.2) methyl 4-(7-acetylamino-3-cyclopropyl-4,5-dihydro-pyrazolo[3′,4′:3,4]benzo[1,2-d]thiazol-1-yl)-2-chloro-benzoate (VIa.2)

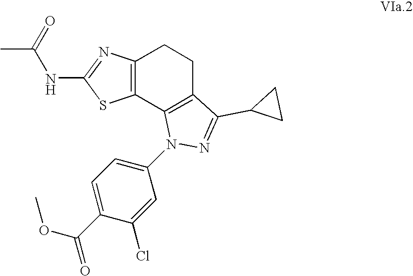

1.50 g (0.539 mol) N-[1-(2-chloro-phenyl)-3-cyclopropyl-4,5-dihydro-1H-pyrazolo[3′,4′:3,4]benzo[1,2-d]thiazol-7-yl]-acetamide, 30 mL glacial acetic acid and 1 mL conc. hydrochloric acid and 1.20 g (0.598 mol) methyl 2-chloro-4-hydrazino-benzoate are used. Yield: 1.22 g (51%).
6.3) methyl 4-(7-acetylamino-3-cyclopropyl-4,5-dihydro-pyrazolo[3′,4′:3,4]benzo[1,2-d]thiazol-1-yl)-2-methoxy-benzoate (VIa.3)

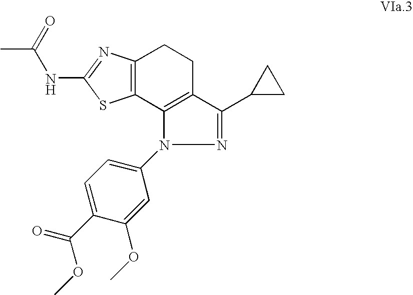

4.00 g (0.0144 mol) N-(6-cyclopropylcarbonyl-7-oxo-4,5,6,7-tetrahydrobenzothiazol-2-yl)-acetamide, 3.40 g (0.0146 mol) methyl 4-hydrazino-2-methoxy-benzoate are used. Yield: 4.70 g (75%)
6.4) methyl 4-[7-acetylamino-3-(1-methyl-cyclopropyl)-4,5-dihydro-pyrazolo[3′,4′:3,4]benzo[1,2-d]thiazol-1-yl]-3-chloro-benzoate (VIa.4)

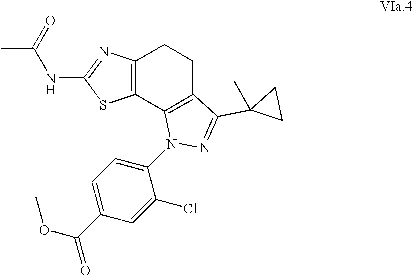

8.70 g (20.83 mmol) N-[6-(1-methyl-cyclopropanecarbonyl)-7-oxo-4,5,6,7-tetrahydro-benzothiazol-2-yl]-acetamide, 7.00 g (34.89 mmol) methyl 3-chloro-4-hydrazino-benzoate and 100 mL glacial acetic acid are used.

Yield: 1.50 g (16%), HPLC-MS: method A, RT=3.22 min, MH+=457/459.
6.5) methyl 4-(7-acetylamino-3-cyclobutyl-4,5-dihydro-pyrazolo[3′,4′:3,4]benzo[1,2-d]thiazol-1-yl)-3-chloro-benzoate (VIa.5)

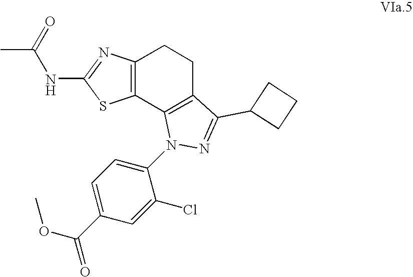

13.30 g (30 mmol) N-(6-cyclobutanecarbonyl-7-oxo-4,5,6,7-tetrahydro-benzothiazol-2-yl)-acetamide, 9.13 g (45 mmol) methyl 3-chloro-4-hydrazino-benzoate and 150 mL glacial acetic acid are used.

Yield: 7.00 g (51%), HPLC-MS: method B, RT=2.15 min, MH+=457.

7) Compounds of Formula VIb

7.1) N-[1-(2-chloro-4-nitro-phenyl)-3-cyclopropyl-4,5-dihydro-1H-pyrazolo[3′,4′:3,4]benzo[1,2-d]thiazol-7-yl]-acetamide (VIb.1)

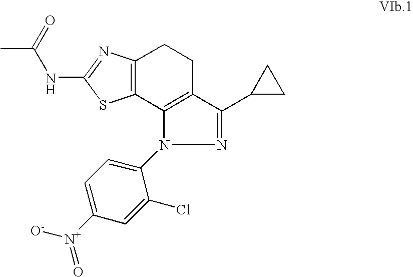

9.00 g (0.0323 mol) N-(6-cyclopropylcarbonyl-7-oxo-4,5,6,7-tetrahydrobenzothiazol-2-yl)-acetamide, 7.24 g (0.0323 mol) (2-chloro-4-nitro-phenyl)-hydrazine hydrochloride and 100 mL glacial acetic acid are used.

Yield: 10.24 g (74%). HPLC-MS: RT=3.09 min, MH+=429/431.

8) Compounds of Formula VII

8.1) 4-(7-acetylamino-3-cyclopropyl-4,5-dihydro-pyrazolo[3′,4′:3,4]benzo[1,2-d]thiazol-1-yl)-3-chloro-benzoic acid

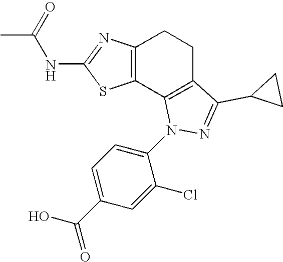

1.60 g (0.00361 mol) methyl 4-(7-acetylamino-3-cyclopropyl-4,5-dihydro-pyrazolo[3′,4′:3,4]benzo[1,2-d]thiazol-1-yl)-3-chloro-benzoate are placed in 40 mL tetrahydrofuran, a solution of 0.500 g (0.0209 mol) lithium hydroxide in 5 mL water is added. The reaction mixture is stirred for 16 h at ambient temperature, then acidified with glacial acetic acid. The solvent is evaporated down i. vac., the residue is mixed with water. Precipitated crystals are suction filtered and washed with water. Yield: 1.51 g (98%).

The following compounds are prepared analogously:
8.2) 4-(7-acetylamino-3-cyclopropyl-4,5-dihydro-pyrazolo[3′,4′:3,4]benzo[1,2-d]thiazol-1-yl)-2-chloro-benzoic acid

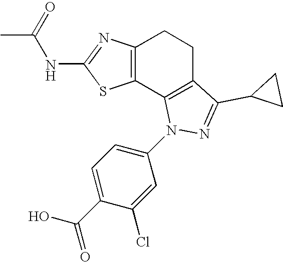

1.20 g (0.00271 mol) methyl 4-(7-acetylamino-3-cyclopropyl-4,5-dihydro-pyrazolo[3′,4′:3,4]benzo[1,2-d]thiazol-1-yl)-2-chloro-benzoate and 0.500 g (0.0209 mol) lithium hydroxide are used. Yield: 1.12 g (96%).
8.3) 4-(7-acetylamino-3-cyclopropyl-4,5-dihydro-pyrazolo[3′,4′:3,4]benzo[1,2-d]thiazol-1-yl)-2-methoxy-benzoic acid

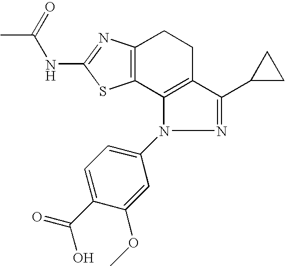

4.70 g (0.0108 mol) methyl 4-(7-acetylamino-3-cyclopropyl-4,5-dihydro-pyrazolo[3′,4′:3,4]benzo[1,2-d]thiazol-1-yl)-2-methoxy-benzoate are used.

Yield: 4.38 g (96%).
8.4) 4-[7-acetylamino-3-(1-methyl-cyclopropyl)-4,5-dihydro-pyrazolo[3′,4′:3,4]benzo[1,2-d]thiazol-1-yl]-3-chloro-benzoic acid

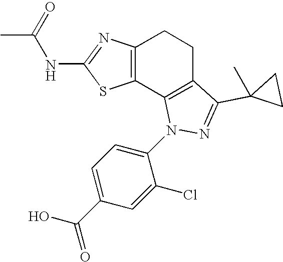

1.50 g (3.28 mmol) methyl 4-[7-acetylamino-3-(1-methyl-cyclopropyl)-4,5-dihydro-pyrazolo[3′,4′:3,4]benzo[1,2-d]thiazol-1-yl]-3-chloro-benzoate in 15 mL dioxane and 0.245 g (10.23 mmol) lithium hydroxide in 1 mL water are used.

Yield: 1.45 g (100%), HPLC-MS: method B, RT=1.93 min, MH+=443/45
8.5) 4-(7-acetylamino-3-cyclobutyl-4,5-dihydro-pyrazolo[3′,4′:3,4]benzo[1,2-d]thiazol-1-yl)-3-chloro-benzoic acid

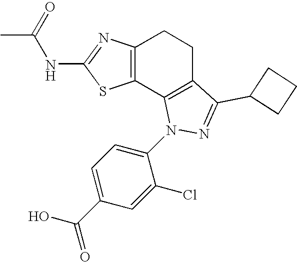

6.70 g (15 mmol) methyl 4-(7-acetylamino-3-cyclobutyl-4,5-dihydro-pyrazolo[3′,4′:3,4]benzo[1,2-d]thiazol-1-yl)-3-chloro-benzoate in 100 mL dioxane and 1.20 g (49.10 mmol) lithium hydroxide in 10 mL water are used.

Yield: 5.80 g (89%), HPLC-MS: method A, RT=3.0 min, MH+=443.

9) Compounds of Formula VIc

9.1) N-(3-cyclopropyl-1-piperidin-4-yl-4,5-dihydro-1H-pyrazolo[3′,4′:3,4]benzo[1,2-d]thiazol-7-yl)-acetamide (VIc.1)

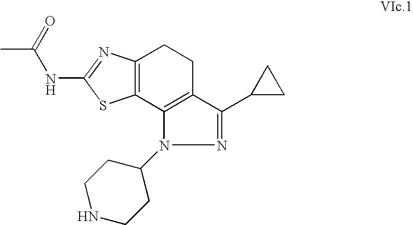

6.12 g (21.99 mmol) N-(6-cyclopropanecarbonyl-7-oxo-4,5,6,7-tetrahydro-benzothiazol-2-yl)-acetamide and 4.30 g (22.86 mmol) piperidin-4-yl-hydrazine are stirred in 50 mL glacial acetic acid for 48 hours at 50° C. Then the mixture is evaporated down, the residue is crystallised from acetonitrile. Yield: 3.00 g (38%).
9.2) N-{3-cyclopropyl-1-[1-(piperidine-4-carbonyl)-piperidin-4-yl]-4,5-dihydro-1H-pyrazolo[3′,4′:3,4]benzo[1,2-d]thiazol-7-yl}-acetamide (VIc.2)

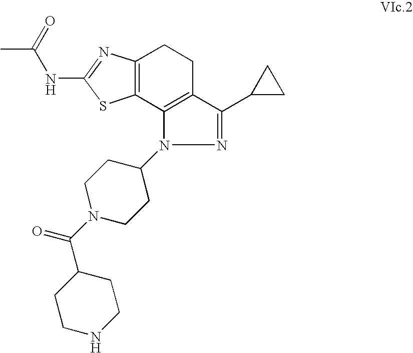

1.00 g (1.76 mmol) tert-butyl 4-[4-(7-acetylamino-3-cyclopropyl-4,5-dihydro-pyrazolo[3′,4′:3,4]benzo[1,2-d]thiazol-1-yl)-piperidine-1-carbonyl]-piperidine-1-carboxylate and 20 mL trifluoroacetic acid are stirred for 24 hours at ambient temperature in 200 mL dichloromethane. The reaction mixture is evaporated down, the residue is made basic with sodium hydroxide solution. The precipitate is suction filtered and dried.

Yield: 0.800 g (97%).

10) Synthesis of Other Intermediate Compounds

10.1) methyl [4-(7-acetylamino-3-cyclopropyl-4,5-dihydro-pyrazolo[3′,4′:3,4]benzo[1,2-d]thiazol-1-yl)-3-chloro-phenyl]-acetate

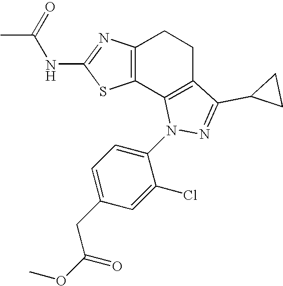

2.70 g (10 mmol) N-(6-cyclopropanecarbonyl-7-oxo-4,5,6,7-tetrahydro-benzothiazol-2-yl)-acetamide, 2.29 g (11 mmol) methyl (3-chloro-4-hydrazino-phenyl)-acetate and 36 mL glacial acetic acid are used. Yield: 2.71 g (61%).
10.2) tert-butyl 4-[4-(7-acetylamino-3-cyclopropyl-4,5-dihydro-pyrazolo[3′,4′:3,4]benzo[1,2-d]thiazol-1-yl)-piperidine-1-carbonyl]-piperidine-1-carboxylate

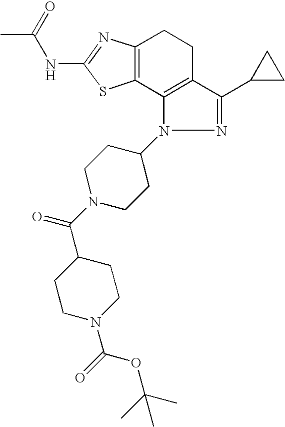

1.00 g (2.80 mmol) N-(3-cyclopropyl-1-piperidin-4-yl-4,5-dihydro-1H-pyrazolo[3′,4′:3,4]benzo[1,2-d]thiazol-7-yl)-acetamide, 0.700 g (3.05 mmol) mono-tert-butyl piperidine-1,4-dicarboxylate, 0.980 g (3.05 mmol) O-(1H-benzotriazol-1-yl)-N,N,N′,N′-tetramethyluroniumtetrafluoroborate (TBTU) and 3 mL triethylamine are stirred in 100 mL dichloromethane for 24 hours at ambient temperature. Then the mixture is extracted with 10% potassium hydrogen carbonate, the organic phase is dried and evaporated to dryness. The residue is crystallised from ethyl acetate. Yield: 1.00 g (63%).
10.3) [4-(7-acetylamino-3-cyclopropyl-4,5-dihydro-pyrazolo[3′,4′:3,4]benzo[1,2-d]thiazol-1-yl)-3-chloro-phenyl]-acetic acid

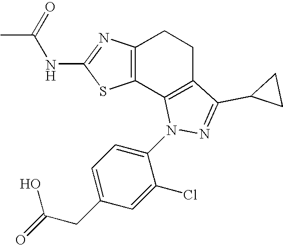

2.70 g (6 mmol) methyl [4-(7-acetylamino-3-cyclopropyl-4,5-dihydro-pyrazolo[3′,4′:3,4]benzo[1,2-d]thiazol-1-yl)-3-chloro-phenyl]-acetate in 95 mL tetrahydrofuran and 0.764 g (31.91 mmol) lithium hydroxide in 10 mL water are used.

Yield: 2.11 g (61%).
10.4) cis-4-(7-acetylamino-3-cyclopropyl-4,5-dihydro-pyrazolo[3′,4′:3,4]benzo[1,2-d]thiazol-1-yl)-cyclohexanecarboxylic acid

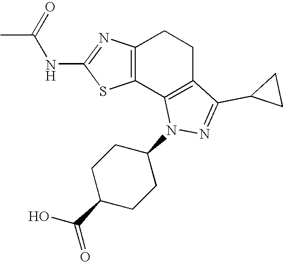

300 mg (0.700 mmol) ethyl cis-4-(7-acetylamino-3-cyclopropyl-4,5-dihydro-pyrazolo[3′,4′:3,4]benzo[1,2-d]thiazol-1-yl)-cyclohexanecarboxylate in 10 mL tetrahydrofuran and 150 mg (6.26 mmol) lithium hydroxide in 2 mL water are used.

Yield: 262 mg (94%).
10.5) trans-4-(7-acetylamino-3-cyclopropyl-4,5-dihydro-pyrazolo[3′,4′:3,4]benzo[1,2-d]thiazol-1-yl)-cyclohexanecarboxylic acid

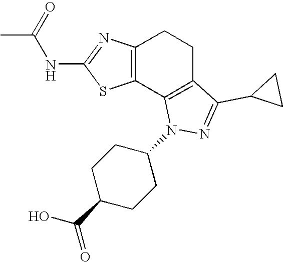

500 mg (1.17 mmol) ethyl trans-4-(7-acetylamino-3-cyclopropyl-4,5-dihydro-pyrazolo[3′,4′:3,4]benzo[1,2-d]thiazol-1-yl)-cyclohexanecarboxylate in 30 mL tetrahydrofuran and 250 mg (10.44 mmol) lithium hydroxide in 10 mL water are used.

Yield: 457mg (98%).
10.6) 4-(7-acetylamino-3-cyclopropyl-4,5-dihydro-pyrazolo[3′,4′:3,4]benzo[1,2-d]thiazol-1-yl)-3-chloro-phenyl-boric acid

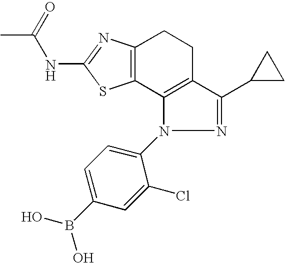

500 mg (0.979 mmol) N-[1-(2-chloro-4-iodo-phenyl)-3-cyclopropyl-4,5-dihydro-1H-pyrazolo[3′,4′:3,4]benzo[1,2-d]thiazol-7-yl]-acetamide are dissolved in 3 mL tetrahydrofuran under a nitrogen atmosphere and under absolutely anhydrous conditions, 41.49 mg (1.00 mmol) lithium chloride are added. The mixture is cooled to −30° C., 0.489 mL (0.979 mmol) isopropylmagnesium chloride in tetrahydrofuran (1 molar) and 0.326 mL (0.979 mmol) methylmagnesium-chloride in tetrahydrofuran (3 molar) are added. The reaction mixture is stirred for 1.5 hours at −10° C. Then 0.51 mL (4.89 mmol) trimethylborate are added dropwise at −20° C. The mixture is stirred for 16 hours at ambient temperature, then combined with 2.5 mL hydrochloric acid (2molar). Water is added to the resulting solution, the tetrahydrofuran is evaporated down in vacuo. The precipitate formed is suction filtered and purified by chromatography. The product is crystallised from ethyl acetate/petroleum ether. Yield: 192.6 mg (46%).
10.7) ethyl cis-4-(7-acetylamino-3-cyclopropyl-4,5-dihydro-pyrazolo[3′,4′:3,4]benzo[1,2-d]thiazol-1-yl)-cyclohexanecarboxylate

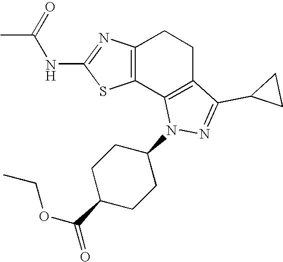

3.00 g (10.78 mmol) N-(6-cyclopropanecarbonyl-7-oxo-4,5,6,7-tetrahydro-benzothiazol-2-yl)-acetamide are placed in 100 mL glacial acetic acid, 2.70 g (12.12 mmol) ethyl cis-4-hydrazino-cyclohexanecarboxylate are added and the mixture is stirred for 72 h at 80° C. Then it is evaporated down, the residue is extracted with ethyl acetate and semiconcentrated ammonia. The organic phase is dried and evaporated to dryness. The residue is purified by chromatography (RP-HPLC).

Yield: 0.317 g (7%), HPLC-MS: method A, RT=3.06 min, MH+=429.
10.8) ethyl trans-4-(7-acetylamino-3-cyclopropyl-4,5-dihydro-pyrazolo[3′,4′:3,4]benzo[1,2-d]thiazol-1-yl)-cyclohexanecarboxylate

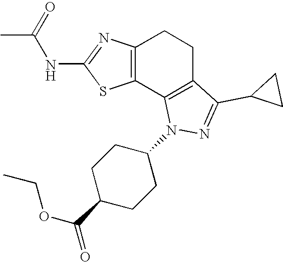

1.95 g (7.01 mmol) N-(6-cyclopropanecarbonyl-7-oxo-4,5,6,7-tetrahydro-benzothiazol-2-yl)-acetamide and 1.60 g (7.18 mmol) ethyl trans-4-hydrazino-cyclohexanecarboxylate are stirred in 100 mL ethanol for 144 hours at 65° C. Then the reaction mixture is evaporated down, the residue is combined with ethyl acetate. Insoluble matter is suction filtered, the filtrate is extracted first with water, then with 5% potassium carbonate solution. The organic phase is dried and evaporated to dryness. The residue is purified by chromatography. Yield: 365 mg (12%).

Synthesis of the Compounds of Formula (I)

The following HPLC-MS methods were used to characterise the compounds of formula (I):
Waters ZMD, Alliance 2690/2695 HPLC, Waters 2700 Autosampler, Waters 996/2996 Diode array detector (wavelength range 210-400 nm).
   Stationary Phase (Column Temperature: Constant at 25° C.):
method A: column XTerra®, MS C₁₈ 2.5 μm, 4.6 mm×30 mm.
method B: column Merck Chromolith™ SpeedROD RP-18e, 4.6 mm×50 mm.
method C: Waters ZQ2000, Gilson 215 Autosampler, HP1100 HPLC+Diode array detector (wavelength range 210-500 nm); column XTerra®, MS C₁₈ 3.5 μm, 4.6 mm×50 mm.
   Mobile Phase:
L1: water with 0.10% TFA; L2: acetonitrile with 0.10% TFA
   Flow Rate:
method A: 1.00 mLl/min
method B: 2.00 mL/min

method C: 1.00 mLl/min

| time (min) | % A | % B |
|---|---|---|
| 0.0 | 95 | 5 |
| 0.1 | 95 | 5 |
| 3.1 | 2 | 98 |
| 4.5 | 2 | 98 |
| 5.0 | 95 | 5 |

The symbol X₂, X₆, etc. used in Tables A to G in the structural formula of the substituent is to be understood as being the linkage point to the remainder of the molecule. The substituent replaces the corresponding groups R², R⁶, etc.

### EXAMPLES

### Example 1

### N-[1-(2-chloro-phenyl)-3-cyclopropyl-4,5-dihydro-1H-pyrazolo[3′,4′:3,4]benzo[1,2-d]thiazol-7-yl]-acetamide

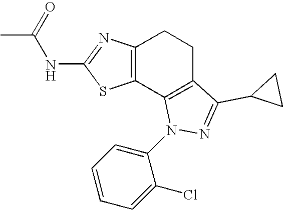

100 mg (0.359 mmol) N-(6-cyclopropylcarbonyl-7-oxo-4,5,6,7-tetrahydrobenzothiazol-2-yl)-acetamide are placed in 5 mL glacial acetic acid, 75 mg (0.419 mmol) o-chlorophenylhydrazine-hydrochloride are added, then the mixture is stirred for 90 h at ambient temperature. Then the reaction mixture is mixed with water and crystallised. Precipitated crystals are suction filtered and recrystallised from acetonitrile. Yield: 86 mg (62%). HPLC-MS: method C, RT=3.91 min, MH+=385.

The following compounds are prepared analogously:

TABLE A

| | |
|---|---|
| | (IA) |
| 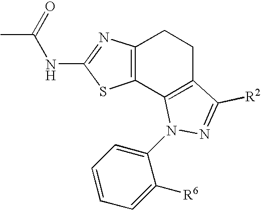 | |

| Ex. | | | | RT | |
|---|---|---|---|---|---|
| A | R² | R⁶ | method | [min] | M + H |
| 1 | 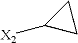 | H | C | 3.88 | 351 |
| 2 | 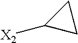 | 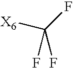 | A | 3.05 | 419 |
| 3 | 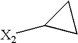 | 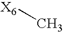 | C | 3.85 | 365 |
| 4 | 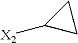 | 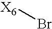 | C | 3.91 | 429 |
| 5 | 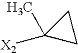 | 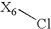 | A | 3.05 | 399/01 |
| 6 | 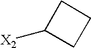 | 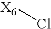 | A | 3.18 | 399 |
| 7 | 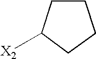 | 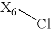 | A | 3.24 | 412 |

### Example 2

### N-[1-(4-amino-2-chloro-phenyl)-3-cyclopropyl-4,5-dihydro-1H-pyrazolo[3′,4′:3,4]benzo[1,2-d]thiazol-7-yl]-acetamide

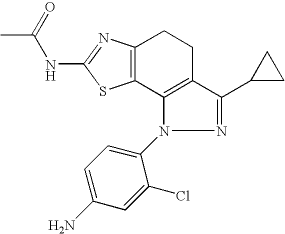

10.24 g (0.0238 mol) N-[1-(2-chloro-4-nitro-phenyl)-3-cyclopropyl-4,5-dihydro-1H-pyrazolo[3′,4′:3,4]benzo[1,2-d]thiazol-7-yl]-acetamide dissolved in 150 mL glacial acetic acid are placed at 70° C. 12.00 g (0.215 mol) iron powder are added. The reaction mixture is stirred for 1.5 hours at 95° C., then suction filtered through kieselguhr and washed with glacial acetic acid. The filtrate is diluted with water. The precipitate formed is suction filtered, washed with water and dried.

The product is purified by chromatography.

Yield: 6.07 g (64%), HPLC-MS: method A, RT=2.54 min, MH+=399.

The following compounds are prepared analogously:

### Example 3

### N-[1-(4-amino-3-chloro-phenyl)-3-cyclopropyl-4,5-dihydro-1H-pyrazolo[3′,4′:3,4]benzo[1,2-d]thiazol-7-yl]-acetamide

3.92 g (9.1 mmol) N-[1-(3-chloro-4-nitro-phenyl)-3-cyclopropyl-4,5-dihydro-1H-pyrazolo[3′,4′:3,4]benzo[1,2-d]thiazol-7-yl]-acetamide and 4.50 g (80.5 mmol) iron powder are used. Yield: 3.6 g (100%), HPLC-MS: method A, RT=3.99 min, MH+=399.

### Example 4

### N-[1-(2-chloro-4-methylamino-phenyl)-3-cyclopropyl-4,5-dihydro-1H-pyrazolo[3′,4′:3,4]benzo[1,2-d]thiazol-7-yl]-acetamide

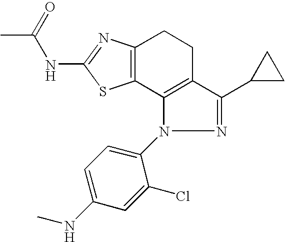

600 mg (1.50 mmol) N-[1-(4-amino-2-chloro-phenyl)-3-cyclopropyl-4,5-dihydro-1H-pyrazolo[3′,4′:3,4]benzo[1,2-d]thiazol-7-yl]-acetamide, 50 mg (1.67 mmol) paraformaldehyde, 700 mg (3 mmol) sodiumacetoxyborohydride and 130 mg (2 mmol) sodium acetate are stirred in 10 mL tetrahydrofuran for 72 h at 60° C. As the reaction is not yet complete, another 5 mg of para-formaldehyde, 200 mg sodium acetoxyborohydride and 40 mg sodium acetate are added and the mixture is stirred for 16 h at 60° C. Then dilute sodium hydrogen carbonate solution is added and the phases are separated. The organic phase is washed with water, dried and evaporated to dryness. The residue is purified by chromatography.

Yield: 160 mg (26%), HPLC-MS: method A, RT=2.73 min, MH+=414/16.

### Example 5

### tert-butyl 4-{[4-(7-acetylamino-3-cyclopropyl-4,5-dihydro-pyrazolo[3′,4′:3,4]benzo[1,2-d]thiazol-1-yl)-3-chloro-phenylamino]-methyl}-piperidine-1-carboxylate

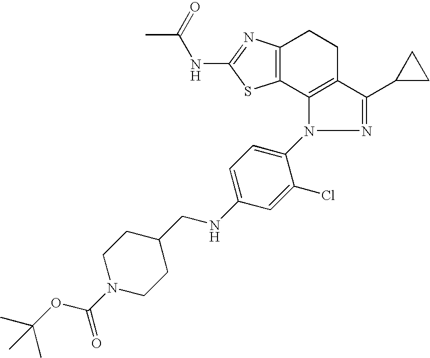

500 mg (1.25 mmol) N-[1-(4-amino-2-chloro-phenyl)-3-cyclopropyl-4,5-dihydro-1H-pyrazolo[3′,4′:3,4]benzo[1,2-d]thiazol-7-yl]-acetamide, 319.90 mg (1.50 mmol) 1-Boc-4-piperidinecarboxaldehyde, 380.50 mg (1.75 mmol) sodium triacetoxyborohydride and 71.50 μL (1.25 mmol) glacial acetic acid are placed in 8 mL dichloroethane and stirred for 16 h at ambient temperature under a nitrogen atmosphere. Then the reaction mixture is mixed with 5% potassium carbonate solution, the organic phase is separated off. The aqueous phase is extracted with dichloromethane. The combined organic phases are dried and evaporated to dryness. The residue is purified by chromatography, suitable fractions are combined, evaporated to dryness and precipitated from ethyl acetate/petroleum ether. Yield: 281.6 mg (38%).

The following compounds are prepared analogously:

### Example 6

### 4-{[4-(7-acetylamino-3-cyclopropyl-4,5-dihydro-pyrazolo[3′,4′:3,4]benzo[1,2-d]thiazol-1-yl)-3-chloro-phenylamino]-methyl}-cyclohexane

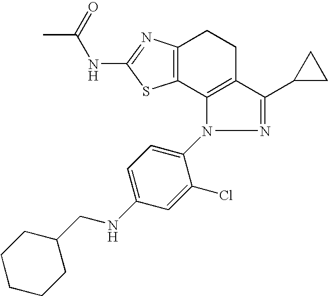

150 mg (0.375 mmol) N-[1-(4-amino-2-chloro-phenyl)-3-cyclopropyl-4,5-dihydro-1H-pyrazolo[3′,4′:3,4]benzo[1,2-d]thiazol-7-yl]-acetamide, 50 μL (0.42 mmol) cyclohexylcarboxaldehyde and 117 mg (0.525 mmol) sodium triacetoxyborohydride are used.

Yield: 93.3 mg (50%); HPLC-MS: method C, RT=3.20 min, MH+=565.

### Example 7

### N-(1-{2-chloro-4-[(piperidin-4-ylmethyl)-amino]-phenyl}-3-cyclopropyl-4,5-dihydro-1H-pyrazolo[3′,4′:3,4]benzo[1,2-d]thiazol-7-yl)-acetamide hydrochloride

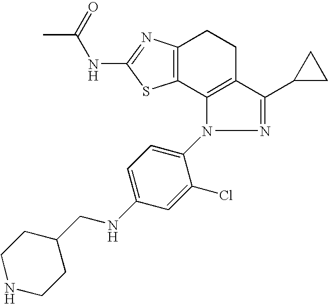

281 mg (0.471 mmol) tert-butyl 4-{[4-(7-acetylamino-3-cyclopropyl-4,5-dihydro-pyrazolo[3′,4′:3,4]benzo[1,2-d]thiazol-1-yl)-3-chloro-phenylamino]-methyl}-piperidine-1-carboxylate and 72.50 μL (0.941 mmol) trifluoroacetic acid are placed in 20 mL dichloromethane, then the mixture is stirred for 16 h at ambient temperature. After the addition of a little phosphate buffer the solution is evaporated down in vacuo. The residue is mixed with water and made basic. The aqueous phase is saturated with sodium chloride and extracted with tetrahydrofuran. The combined organic phases are dried and evaporated to dryness. The hydrochloride is precipitated.

Yield: 236 mg (94%). HPLC-MS: method C, RT=3.18 min, MH+=497.

### Example 8

### N-(1-{2-chloro-4-[(1-cyclopentyl-piperidin-4-ylmethyl)-amino]-phenyl}-3-cyclopropyl-4,5-dihydro-1H-pyrazolo[3′,4′:3,4]benzo[1,2-d]thiazol-7-yl)-acetamide trifluoroacetate

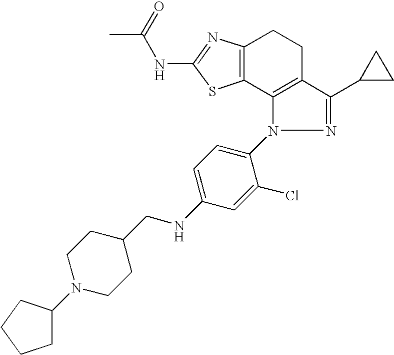

128 mg (0.240 mmol) N-(1-{2-chloro-4-[(piperidin-4-ylmethyl)-amino]-phenyl}-3-cyclopropyl-4,5-dihydro-1H-pyrazolo[3′,4′:3,4]benzo[1,2-d]thiazol-7-yl)-acetamide hydrochloride, 25.49 μL (0.288 mmol) cyclopentanone, 74.93 mg (0.336 mmol) sodium triacetoxyborohydride and 13.72 μL (0.240 mmol) glacial acetic acid are stirred in 6 mL dichloroethane for 48 hours at ambient temperature. The reaction mixture is combined with 5% potassium carbonate solution and the phases are separated. The aqueous phase is extracted with dichloromethane, the combined organic phases are dried and evaporated to dryness. The residue is dissolved in acetonitrile, water and trifluoroacetic acid and purified by chromatography.

Yield: 40.10 mg (25%). HPLC-MS: method C, RT=3.39 min, MH+=565.

The following compounds are prepared analogously:

### Example 9

### N-(1-{2-chloro-4-[(1-methyl-piperidin-4-ylmethyl)-amino]-phenyl}-3-cyclopropyl-4,5-dihydro-1H-pyrazolo[3′,4′:3,4]benzo[1,2-d]thiazol-7-yl)-acetamide trifluoroacetate

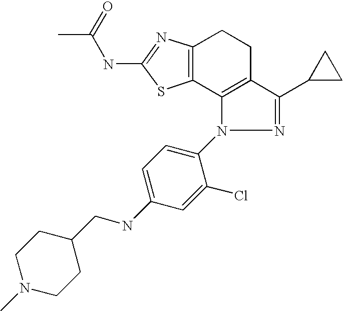

150 mg (0.240 mmol) N-(1-{2-chloro-4-[(piperidin-4-ylmethyl)-amino]-phenyl}-3-cyclopropyl-4,5-dihydro-1H-pyrazolo[3′,4′:3,4]benzo[1,2-d]thiazol-7-yl)-acetamide hydrochloride, 47.5 μL (0.6 mmol) formaldehyde (37%) and 141.4 mg (0.64 mmol) sodium triacetoxyborohydride are used.

Yield: 204 mg (92%); HPLC-MS: method C, RT=3.22 min, MH+=511.

### Example 10

### N-(1-{2-chloro-4-[(1-propyl-piperidin-4-ylmethyl)-amino]-phenyl}-3-cyclopropyl-4,5-dihydro-1H-pyrazolo[3′,4′:3,4]benzo[1,2-d]thiazol-7-yl)-acetamide trifluoroacetate

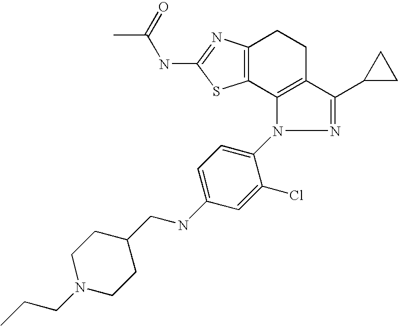

150 mg (0.240 mmol) N-(1-{2-chloro-4-[(piperidin-4-ylmethyl)-amino]-phenyl}-3-cyclopropyl-4,5-dihydro-1H-pyrazolo[3′,4′:3,4]benzo[1,2-d]thiazol-7-yl)-acetamide hydrochloride, 26 μL (0.36 mmol) propionaldehyde and 134.7 mg (0.6 mmol) sodium triacetoxyborohydride are used.

Yield: 103 mg (44%); HPLC-MS: method C, RT=3.32 min, MH+=539.

### Example 11

### 1-cyclopentyl-piperidine-4-carboxylic acid[4-(7-acetylamino-3-cyclopropyl-4,5-dihydro-pyrazolo[3′,4′:3,4]benzo[1,2-d]thiazol-1-yl)-3-chloro-phenyl]-amide

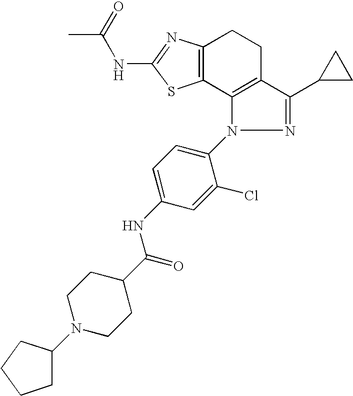

100 mg (0.250 mmol) N-[1-(4-amino-2-chloro-phenyl)-3-cyclopropyl-4,5-dihydro-1H-pyrazolo[3′,4′:3,4]benzo[1,2-d]thiazol-7-yl]-acetamide, 96 μL (0.300 mmol) TBTU and 100 μL (0.721 mmol) triethylamine are placed in 5 mL dichloromethane, and the mixture is stirred for 0.5 h at ambient temperature. 70.10 mg (0.278 mmol) 1-cyclopentyl-piperidine-4-carbonyl chloride are added, then the mixture is stirred for 16 hours at ambient temperature. Then the reaction mixture is diluted with dichloromethane and extracted with 5% potassium carbonate solution. The phases are separated using a phase separation cartridge, the aqueous phase is again extracted with dichloromethane. The combined organic phases are dried and evaporated to dryness. The residue is purified by chromatography. Corresponding fractions are combined and evaporated down. The product is crystallised from ethyl acetate/petroleum ether, then again purified by chromatography (HPLC) and lyophilised. Yield: 48.20 mg (28%), HPLC-MS: method A, RT=2.53 min, MH+=579.

### Example 12

### 1-propyl-piperidine-4-carboxylic acid[4-(7-acetylamino-3-cyclopropyl-4,5-dihydro-pyrazolo[3′,4′:3,4]benzo[1,2-d]thiazol-1-yl)-3-chloro-phenyl]-methyl-amide

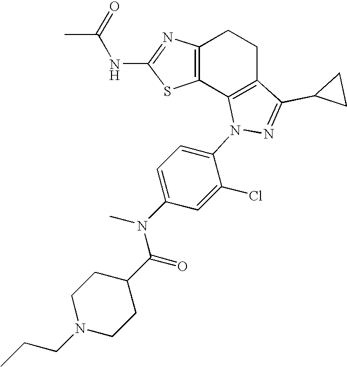

50 mg (0.121 mmol) N-[1-(2-chloro-4-methylamino-phenyl)-3-cyclopropyl-4,5-dihydro-1H-pyrazolo[3′,4′:3,4]benzo[1,2-d]thiazol-7-yl]-acetamide and 60 mg (0.265 mmol) 1-propyl-piperidine-4-carbonyl chloride are stirred in 1.50 mL pyridine for 16 h at ambient temperature. Then the reaction mixture is extracted with dichloromethane and dilute potassium carbonate solution. The organic phase is evaporated down with Extrelut, then purified by chromatography. Corresponding fractions are combined and evaporated down. The residue is crystallised from ethyl acetate/n-heptane.

Yield: 25 mg (36%), HPLC-MS: method A, RT=2.51 min, MH+567/69.

The following compounds are prepared analogously:

TABLE B

| | |
|---|---|
| | (IB) |
| 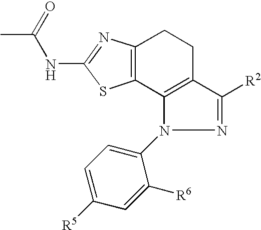 | |

| Ex | | | | | RT | |
|---|---|---|---|---|---|---|
| B | R² | R⁶ | R⁵ | method | [min] | [M + H]+ |
| 1 | 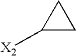 | 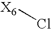 | 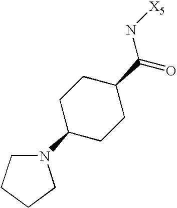 | A | 2.50 | 579 |
| 2 | 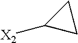 | 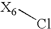 | 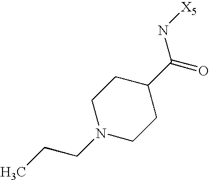 | A | 2.43 | 553/55 |
| 3 | 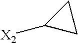 | 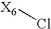 | 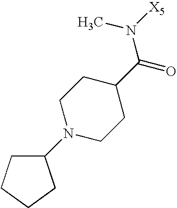 | A | 2.59 | 593/95 |
| 4 | 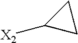 | 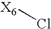 | 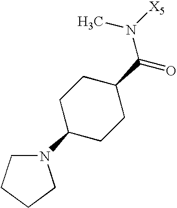 | A | 2.45 | 593/95 |

### Example 13

### 4-(7-acetylamino-3-cyclopropyl-4,5-dihydro-pyrazolo[3′,4′:3,4]benzo[1,2-d]thiazol-1-yl)-3-chloro-N-methyl-N-(cis-4-pyrrolidin-1-yl-cyclohexyl)-benzamide

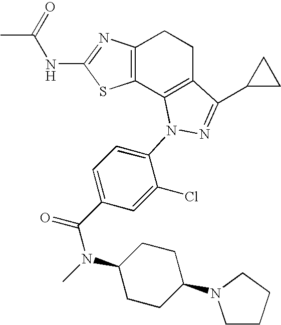

80 mg (0.187 mmol) 4-(7-acetylamino-3-cyclopropyl-4,5-dihydro-pyrazolo[3′,4′:3,4]benzo[1,2-d]thiazol-1-yl)-3-chloro-benzoic acid are placed in 5 mL dichloromethane, 70 mg (0.218 mmol) O-(1H-benzotriazol-1-yl)-N,N,N′,N′-tetramethyluroniumtetrafluoroborate and 0.15 mL (1.08 mmol) triethylamine are added. The mixture is stirred for 15 min. at ambient temperature, then 50 mg (0.196 mmol) methyl-(cis-4-pyrrolidin-1-yl-cyclohexyl)-amine are added. The reaction mixture is stirred for 16 h at ambient temperature, then diluted with dichloromethane and extracted with 5% potassium carbonate solution. The organic phase is separated off using a phase separation cartridge and evaporated to dryness. The residue is crystallised from ethyl acetate. Yield: 70 mg (63%).HPLC-MS: method A, RT=2.49 min, MH+593/5.

The following compounds are prepared analogously:

TABLE C

| | |
|---|---|
| | (IC) |
| 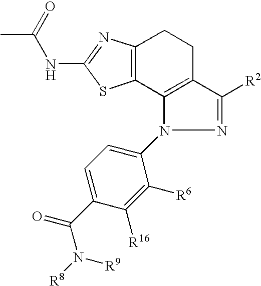 | |

| Ex | | | | | | RT | |
|---|---|---|---|---|---|---|---|
| C | R² | R⁶ | R¹⁶ | NR⁸R⁹(═R²⁴) | method | [min] | M + H |
| 1 | 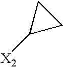 | 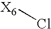 | H | 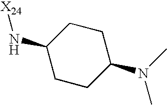 | C | 3.23 | 553 |
| 2 | 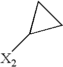 | 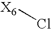 | H | 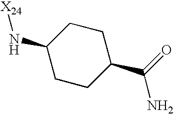 | C | 3.5 | 553 |
| 3 | 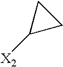 | 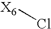 | H | 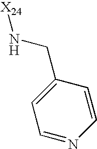 | C | 3.18 | 519 |
| 4 | 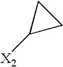 | 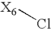 | H | 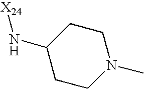 | C | 3.17 | 525 |
| 5 | 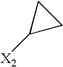 | 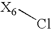 | H | 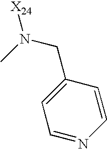 | C | 3.18 | 533 |
| 6 | 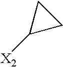 | 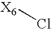 | H | 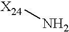 | C | 3.46 | 428 |
| 7 | 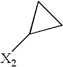 | 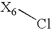 | H | 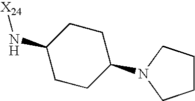 | C | 3.26 | 579 |
| 8 | 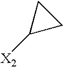 | 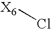 | H | 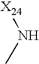 | C | 3.19 | 565 |
| 9 | 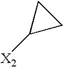 | 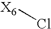 | H | 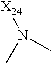 | C | 3.32 | 593 |
| 10 | 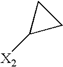 | 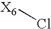 | H | 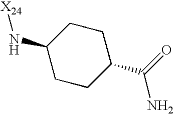 | C | 3.48 | 553 |
| 11 | 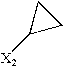 | 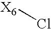 | H | 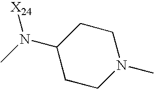 | C | 3.17 | 539 |
| 12 | 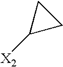 | 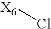 | H | 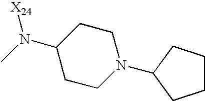 | C | 3.34 | 593 |
| 13 | 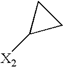 | 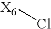 | H | 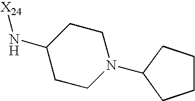 | | | |
| 14 | 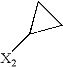 | 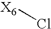 | H | 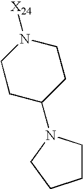 | C | 3.19 | 565 |
| 15 | 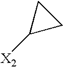 | 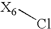 | H | 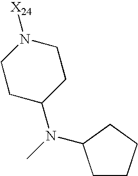 | C | 3.32 | 593 |
| 16 | 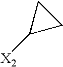 | H | 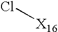 | 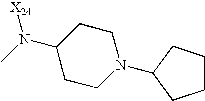 | C | 3.39 | 593 |
| 17 | 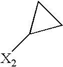 | H | 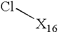 | 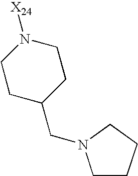 | C | 3.28 | 579 |
| 18 | 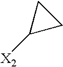 | H | 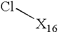 | 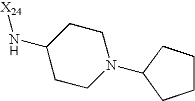 | C | 3.35 | 579 |
| 19 | 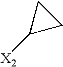 | H | 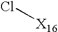 | 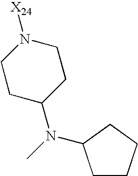 | C | 3.36 | 593 |
| 20 | 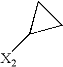 | H | 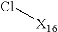 | 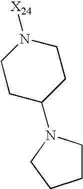 | C | 3.24 | 565 |
| 21 | 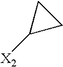 | H | 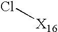 | 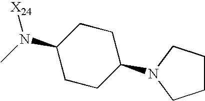 | C | 3.28 | 579 |
| 22 | 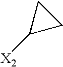 | H | 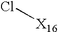 | 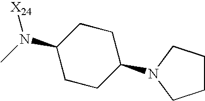 | C | 3.33 | 593 |
| 23 | 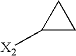 | H | 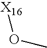 | 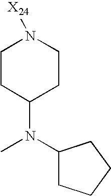 | A | 2.45 | 589 |
| 24 | 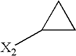 | H | 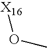 | 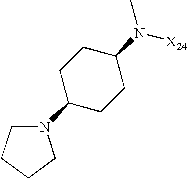 | A | 2.42 | 589 |
| 25 | 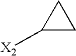 | H | 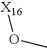 | 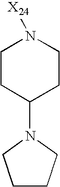 | A | 2.35 | 561 |
| 26 | 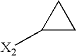 | H | 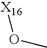 | 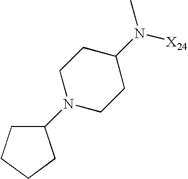 | A | 2.46 | 589 |
| 27 | 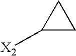 | H | 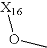 | 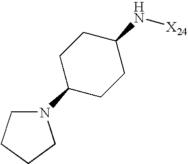 | A | 2.46 | 575 |
| 28 | 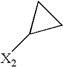 | H | 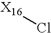 | 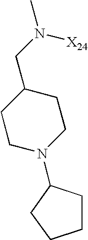 | C | 3.44 | 607 |
| 29 | 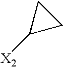 | 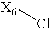 | | 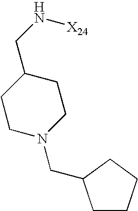 | C | 3.44 | 607 |
| 30 | 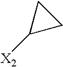 | H | 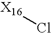 | 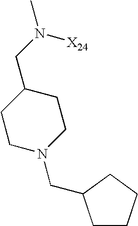 | C | 3.54 | 621 |
| 31 | 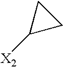 | H | 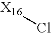 | 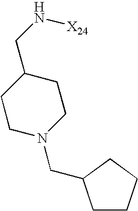 | C | 3.47 | 607 |
| 32 | 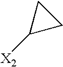 | 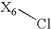 | | 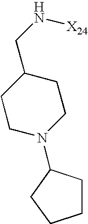 | C | 3.34 | 593 |
| 33 | 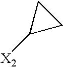 | H | 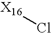 | 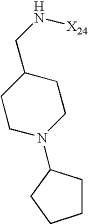 | C | 3.37 | 593 |
| 34 | 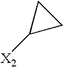 | 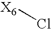 | | 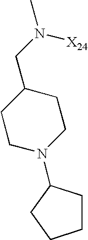 | C | 3.37 | 607 |
| 35 | 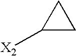 | H | 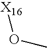 | 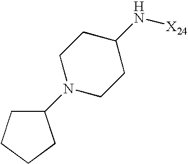 | C | 3.31 | 575 |
| 36 | 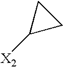 | H | 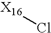 | 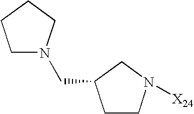 | C | 3.25 | 565 |
| 37 | 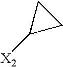 | H | 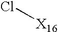 | 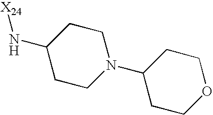 | C | 3.22 | 595 |
| 38 | 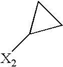 | H | 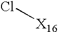 | 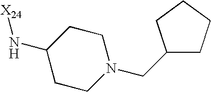 | C | 3.45 | 593 |
| 39 | 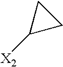 | H | 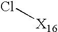 | 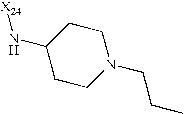 | C | 3.29 | 553 |
| 40 | 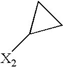 | H | 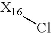 | 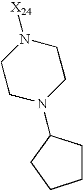 | C | 3.35 | 565 |
| 41 | 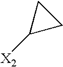 | H | 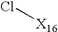 | 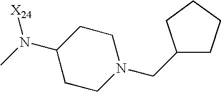 | C | 3.48 | 607 |
| 42 | 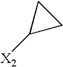 | 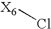 | H | 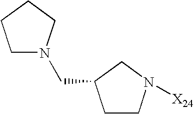 | C | 3.2 | 565 |
| 43 | 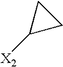 | H | 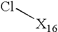 | 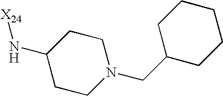 | C | 3.52 | 607 |
| 44 | 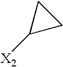 | H | 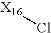 | 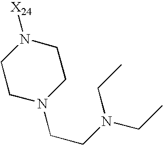 | C | 3.12 | 596 |
| 45 | 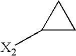 | H | 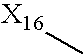 | 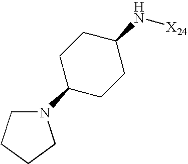 | A | 2.46 | 559 |
| 46 | 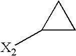 | H | 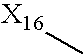 | 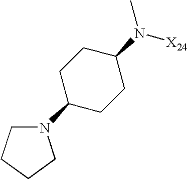 | A | 2.46 | 573 |
| 47 | 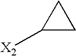 | H | 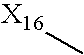 | 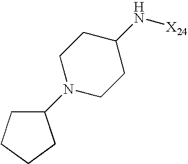 | A | 2.51 | 559 |
| 48 | 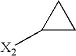 | H | 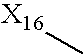 | 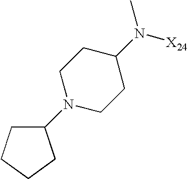 | A | 2.52 | 573 |
| 49 | 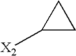 | H | 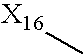 | 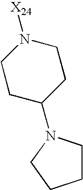 | A | 2.3 | 545 |
| 50 | 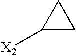 | H | 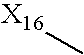 | 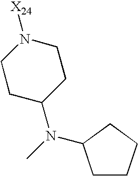 | A | 2.45 | 573 |
| 51 | 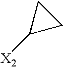 | H | 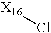 | 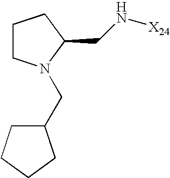 | C | 3.6 | 593 |
| 52 | 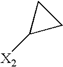 | H | 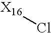 | 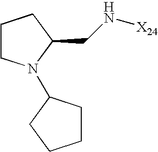 | C | 3.47 | 579 |
| 53 | 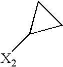 | H | 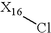 | 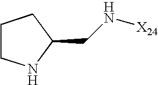 | C | 3.21 | 511 |
| 54 | 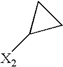 | H | 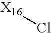 | 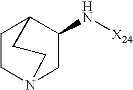 | A | 2.47 | 537 |
| 55 | 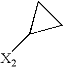 | H | 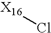 | 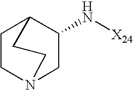 | A | 2.47 | 537 |
| 56 | 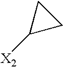 | H | 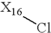 | 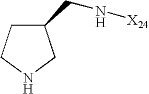 | C | 3.16 | 511 |
| 57 | 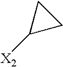 | H | 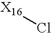 | 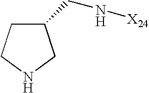 | C | 3.17 | 511 |
| 58 | 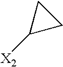 | H | 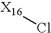 | 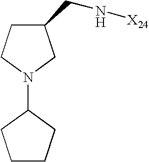 | C | 3.37 | 579 |
| 59 | 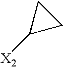 | H | 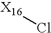 | 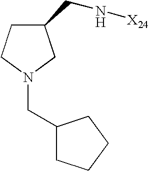 | C | 3.47 | 593 |
| 60 | 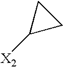 | H | 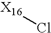 | 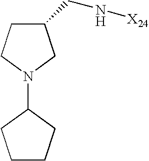 | C | 3.37 | 579 |
| 61 | 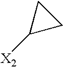 | H | 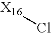 | 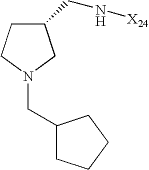 | C | 3.47 | 593 |
| 62 | 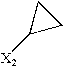 | H | 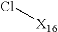 | 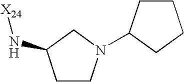 | C | 3.37 | 565 |
| 63 | 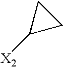 | H | 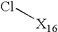 | 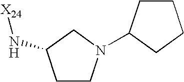 | C | 3.36 | 565 |
| 64 | 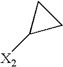 | H | 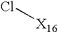 | 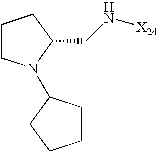 | C | 3.46 | 579 |
| 65 | 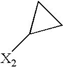 | H | 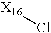 | 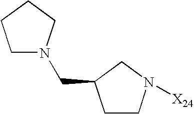 | C | 3.26 | 565 |
| 66 | 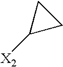 | H | 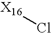 | 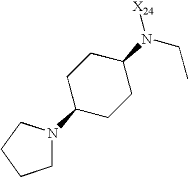 | A | 2.56 | 607 |
| 67 | 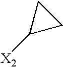 | H | 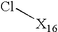 | 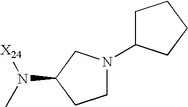 | C | 3.43 | 579 |
| 68 | 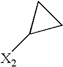 | H | 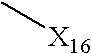 | 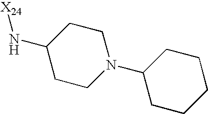 | C | 3.36 | 573 |
| 69 | 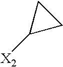 | H | 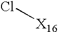 | 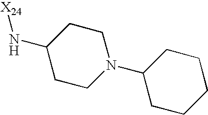 | C | 3.42 | 593 |
| 70 | 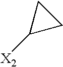 | H | 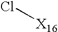 | 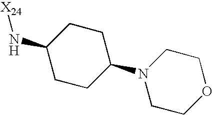 | C | 3.24 | 595 |
| 71 | 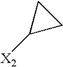 | H | 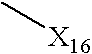 | 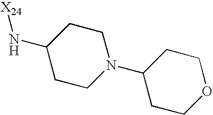 | C | 3.17 | 575 |
| 72 | 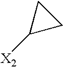 | H | 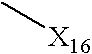 | 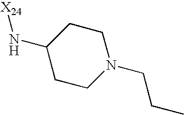 | C | 3.23 | 533 |
| 73 | 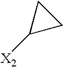 | H | 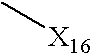 | 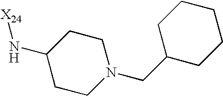 | C | 3.46 | 587 |
| 74 | 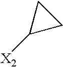 | H | 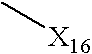 | 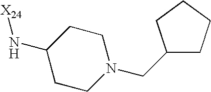 | C | 3.39 | 573 |
| 75 | 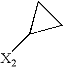 | H | 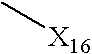 | 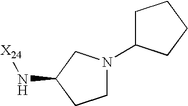 | C | 3.32 | 545 |
| 76 | 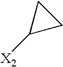 | H | 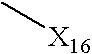 | 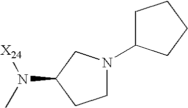 | C | 3.35 | 559 |
| 77 | 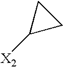 | H | 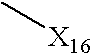 | 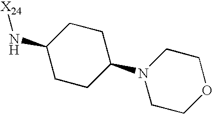 | C | 3.18 | 575 |
| 78 | 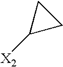 | H | 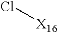 | 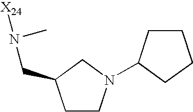 | C | 3.43 | 593 |
| 79 | 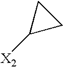 | 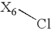 | H | 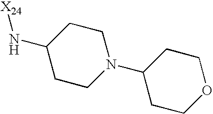 | C | 3.21 | 595 |
| 80 | 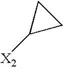 | 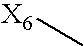 | H | 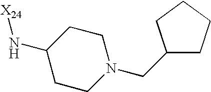 | A | 2.55 | 573 |
| 81 | 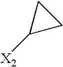 | 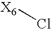 | H | 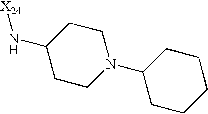 | C | 3.38 | 593 |
| 82 | 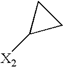 | 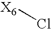 | H | 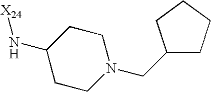 | C | 3.43 | 593 |
| 83 | 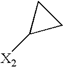 | 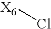 | H | 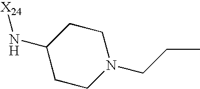 | C | 3.27 | 553 |
| 84 | 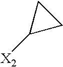 | 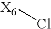 | H | 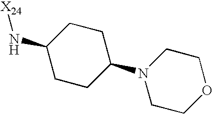 | C | 3.22 | 595 |
| 85 | 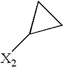 | 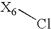 | H | 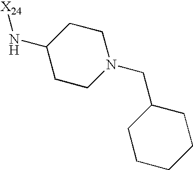 | C | 3.5 | 607 |
| 86 | 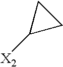 | 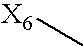 | H | 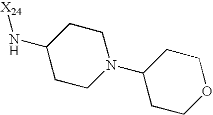 | C | 3.17 | 575 |
| 87 | 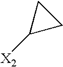 | 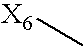 | H | 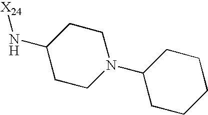 | C | 3.34 | 573 |
| 88 | 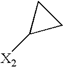 | 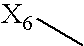 | H | 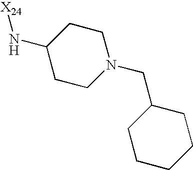 | C | 3.45 | 587 |
| 89 | 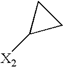 | 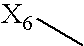 | H | 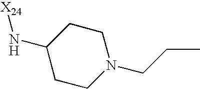 | A | 2.42 | 532 |
| 90 | 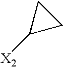 | 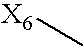 | H | 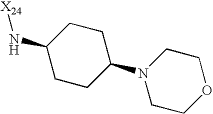 | C | 3.19 | 575 |
| 91 | 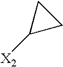 | 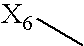 | H | 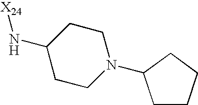 | A | 2.48 | 558 |
| 92 | 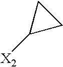 | 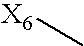 | H | 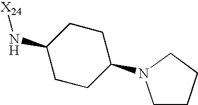 | A | 2.4 | 558 |
| 93 | 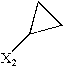 | 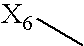 | H | 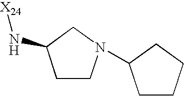 | C | 3.32 | 545 |
| 94 | 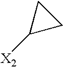 | 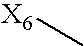 | H | 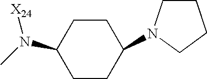 | C | 3.24 | 573 |
| 95 | 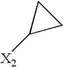 | 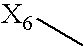 | H | 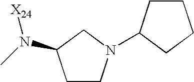 | C | 3.32 | 559 |
| 96 | 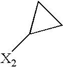 | 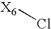 | H | 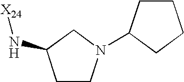 | A | 2.60 | 565 |
| 97 | 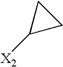 | 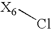 | H | 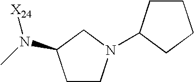 | A | 2.58 | 579 |
| 98 | 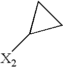 | 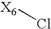 | H | 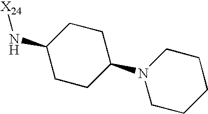 | A | 2.57 | 593 |
| 99 | 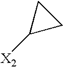 | H | 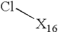 | 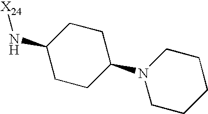 | A | 2.54 | 593 |
| 100 | 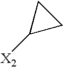 | H | 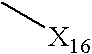 | 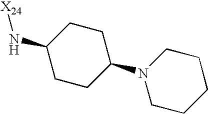 | A | 2.49 | 572 |
| 101 | 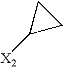 | 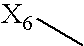 | H | 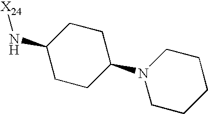 | A | 2.48 | 572 |
| 102 | 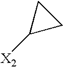 | 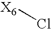 | H | 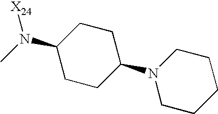 | A | 2.55 | 607 |
| 103 | 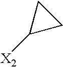 | H | 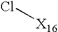 | 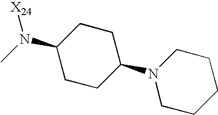 | A | 2.57 | 607 |
| 104 | 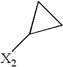 | H | 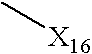 | 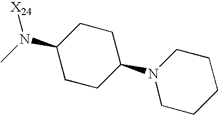 | A | 2.49 | 586 |
| 105 | 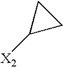 | 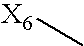 | H | 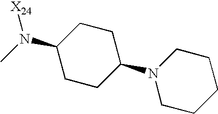 | A | 2.48 | 586 |
| 106 | 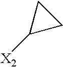 | 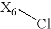 | H | 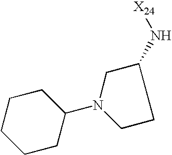 | C | 3.4 | 579 |
| 107 | 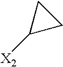 | H | 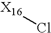 | 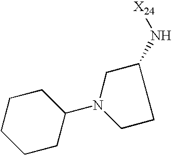 | C | 3.45 | 579 |
| 108 | 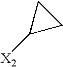 | H | 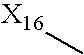 | 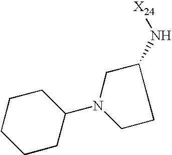 | C | 3.4 | 559 |
| 109 | 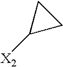 | 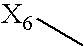 | H | 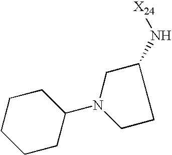 | | | |
| 110 | 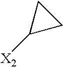 | 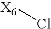 | H | 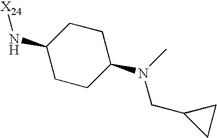 | C | 3.35 | 593 |
| 111 | 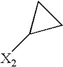 | 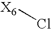 | H | 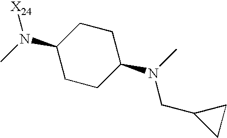 | A | 2.56 | 607 |
| 112 | 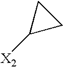 | H | 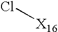 | 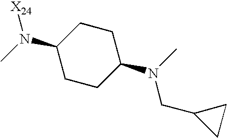 | C | 3.41 | 607 |
| 113 | 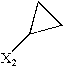 | H | 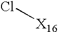 | 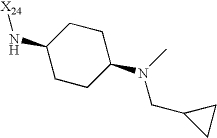 | C | 3.35 | 593 |
| 114 | 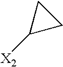 | H | 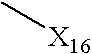 | 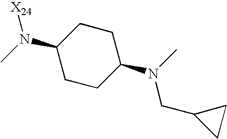 | C | 3.32 | 587 |
| 115 | 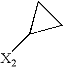 | 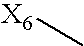 | H | 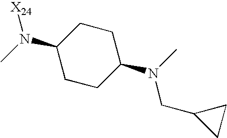 | C | 3.32 | 587 |
| 116 | 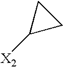 | 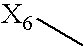 | H | 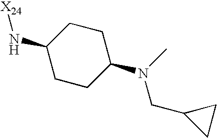 | C | 3.3 | 573 |
| 117 | 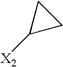 | H | 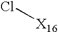 | 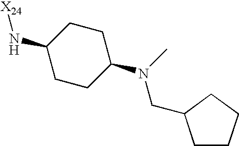 | C | 3.52 | 621 |
| 118 | 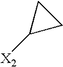 | H | 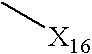 | 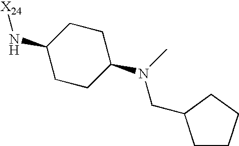 | C | 3.46 | 601 |
| 119 | 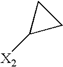 | 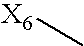 | H | 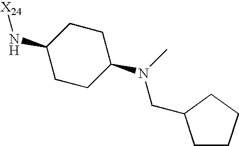 | C | 3.46 | 601 |
| 120 | 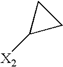 | 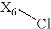 | H | 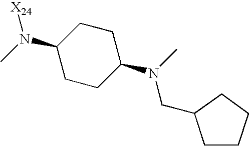 | C | 3.49 | 635 |
| 121 | 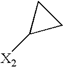 | H | 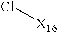 | 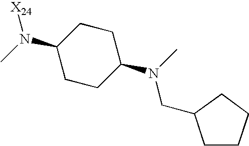 | C | 3.56 | 635 |
| 122 | 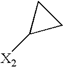 | H | 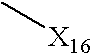 | 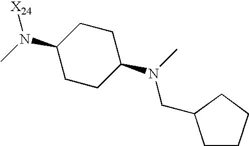 | C | 3.46 | 615 |
| 123 | 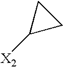 | 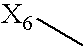 | H | 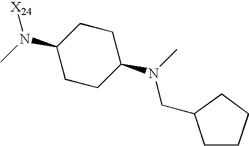 | C | 3.45 | 615 |
| 124 | 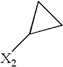 | 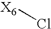 | H | 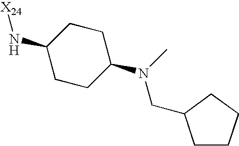 | A | 2.73 | 621 |
| 125 | 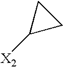 | H | 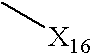 | 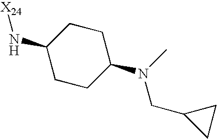 | C | 3.31 | 573 |
| 126 | 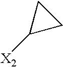 | 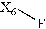 | H | 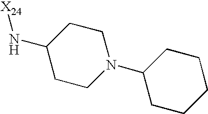 | A | 2.54 | 576 |
| 127 | 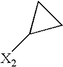 | 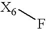 | H | 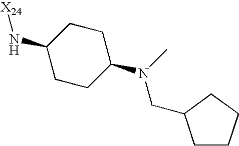 | A | 2.7 | 604 |
| 128 | 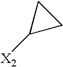 | 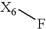 | H | 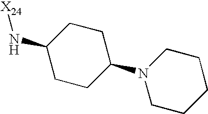 | A | 2.53 | 576 |
| 129 | 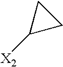 | 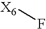 | H | 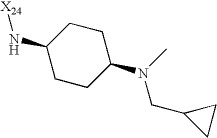 | A | 2.5 | 576 |
| 130 | 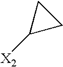 | 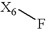 | H | 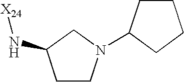 | A | 2.51 | 248 |
| 131 | 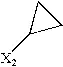 | H | 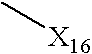 | 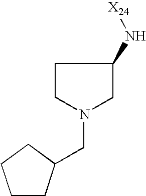 | C | 3.45 | 559 |
| 132 | 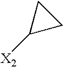 | 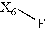 | H | 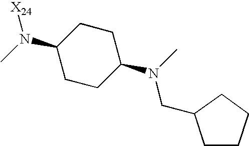 | A | 2.66 | 618 |
| 133 | 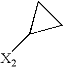 | 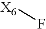 | H | 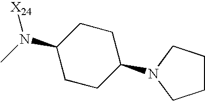 | A | 2.44 | 576 |
| 134 | 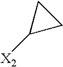 | 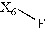 | H | 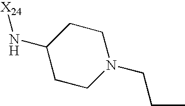 | A | 2.48 | 536 |
| 135 | 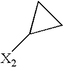 | 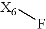 | H | 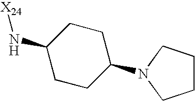 | A | 2.48 | 563 |
| 136 | 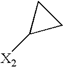 | 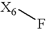 | H | 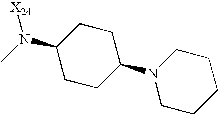 | A | 2.53 | 590 |
| 137 | 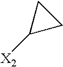 | 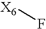 | H | 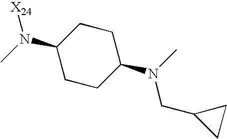 | A | 2.55 | 590 |
| 138 | 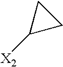 | H | 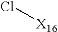 | 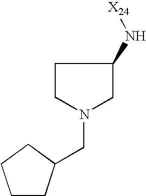 | A | 2.72 | 579 |
| 139 | 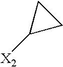 | 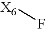 | H | 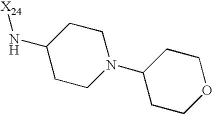 | A | 2.44 | 578 |
| 140 | 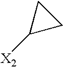 | 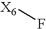 | H | 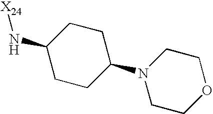 | A | 2.42 | 578 |
| 141 | 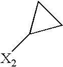 | 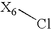 | H | 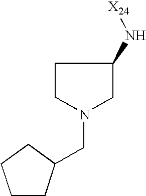 | A | 2.66 | 579 |
| 142 | 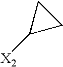 | 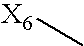 | H | 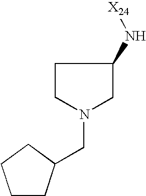 | C | 3.44 | 559 |
| 143 | 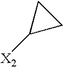 | 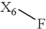 | H | 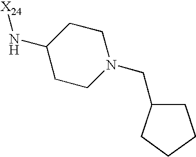 | A | 2.56 | 576 |
| 144 | 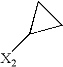 | 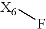 | H | 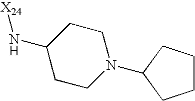 | A | 2.48 | 563 |
| 145 | 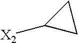 | H | 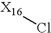 | 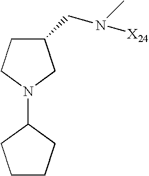 | A | 2.56 | 593 |
| 146 | 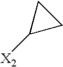 | 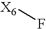 | H | 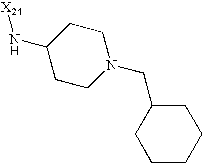 | A | 2.7 | 590 |
| 147 | 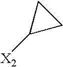 | H | 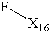 | 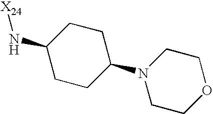 | A | 2.49 | 579 |
| 148 | 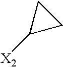 | H | 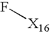 | 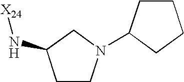 | A | 2.59 | 548 |
| 149 | 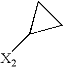 | H | 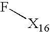 | 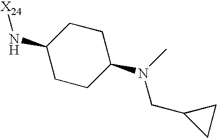 | A | 2.59 | 576 |
| 150 | 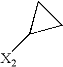 | H | 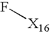 | 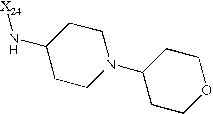 | A | 2.49 | 579 |
| 151 | 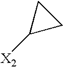 | H | 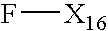 | 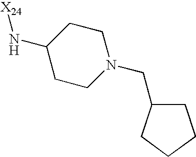 | A | 2.64 | 577 |
| 152 | 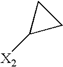 | H | 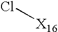 | 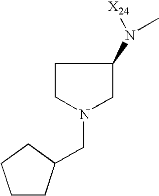 | A | 2.76 | 593 |
| 153 | 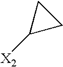 | H | 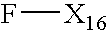 | 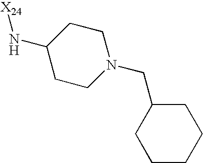 | A | 2.72 | 591 |
| 154 | 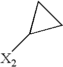 | H | 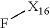 | 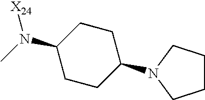 | A | 2.55 | 576 |
| 155 | 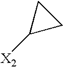 | H | 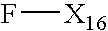 | 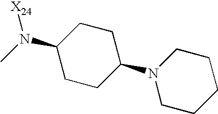 | A | 2.55 | 591 |
| 156 | 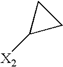 | H | 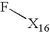 | 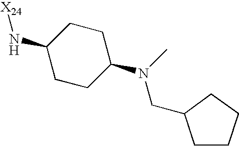 | A | 2.69 | 604 |
| 157 | 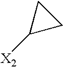 | H | 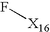 | 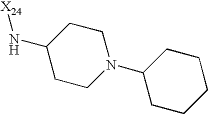 | A | 2.62 | 576 |
| 158 | 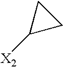 | H | 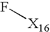 | 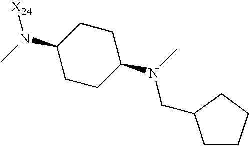 | A | 2.75 | 618 |
| 159 | 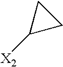 | H | 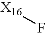 | 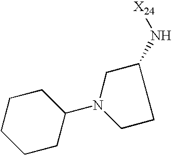 | A | 2.62 | 562 |
| 160 | 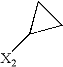 | H | 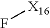 | 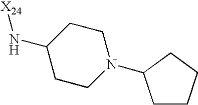 | A | 2.55 | 562 |
| 161 | 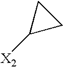 | H | 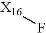 | 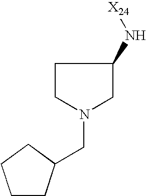 | A | 2.68 | 562 |
| 162 | 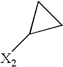 | 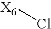 | H | 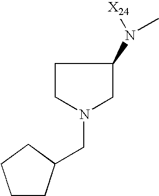 | A | 2.72 | 593 |
| 163 | 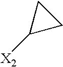 | H | 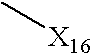 | 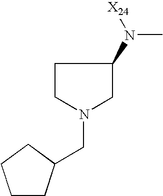 | A | 2.66 | 572 |
| 164 | 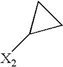 | 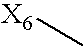 | H | 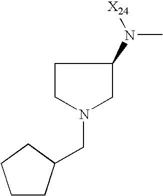 | A | 2.63 | 573 |
| 165 | 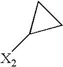 | 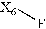 | H | 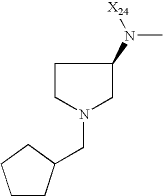 | C | 3.46 | 577 |
| 166 | 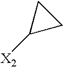 | H | 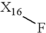 | 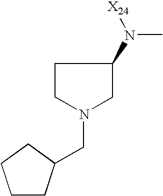 | C | 3.53 | 577 |
| 167 | 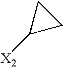 | 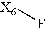 | H | 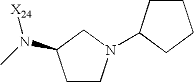 | A | 2.46 | 562 |
| 168 | 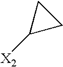 | 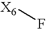 | H | 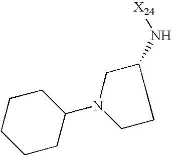 | A | 2.56 | 562 |
| 169 | 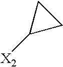 | H | 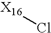 | 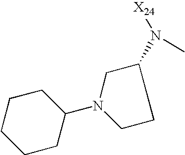 | A | 2.68 | 593 |
| 170 | 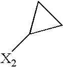 | H | 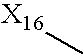 | 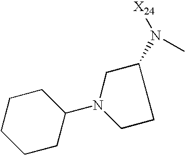 | A | 2.62 | 572 |
| 171 | 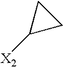 | 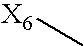 | H | 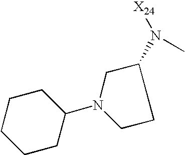 | A | 2.59 | 572 |
| 172 | 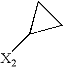 | H | 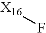 | 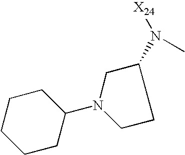 | A | 2.63 | 576 |
| 173 | 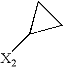 | H | 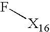 | 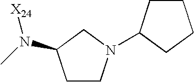 | A | 2.56 | 562 |
| 174 | 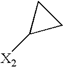 | H | 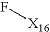 | 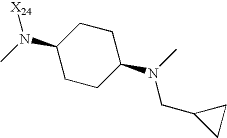 | A | 2.59 | 590 |
| 175 | 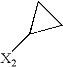 | 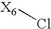 | H | 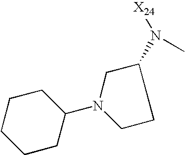 | A | 2.68 | 593 |
| 176 | 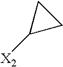 | 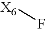 | H | 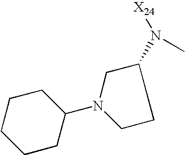 | A | 2.61 | 576 |
| 177 | 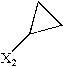 | H | 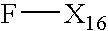 | 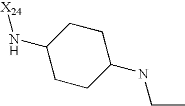 | A | 2.49 | 537 |
| 178 | 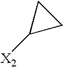 | H | 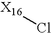 | 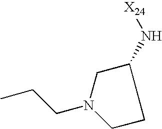 | A | 2.38 | 539 |
| 179 | 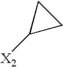 | H | 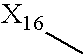 | 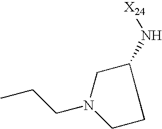 | A | 2.38 | 518 |
| 180 | 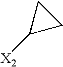 | 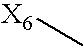 | H | 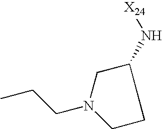 | A | 2.48 | 518 |
| 181 | 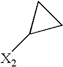 | H | 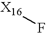 | 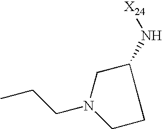 | A | 2.48 | 522 |
| 182 | 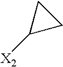 | 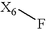 | H | 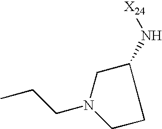 | A | 2.45 | 522 |
| 183 | 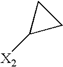 | 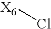 | H | 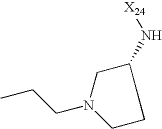 | A | 2.48 | 539 |
| 184 | 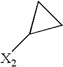 | 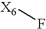 | H | 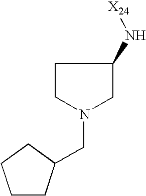 | A | 2.63 | 562 |
| 185 | 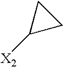 | H | 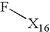 | 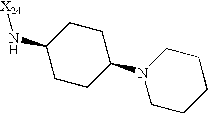 | A | 2.49 | 576 |
| 186 | 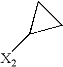 | 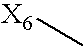 | H | 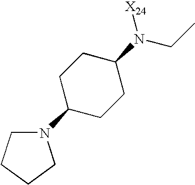 | A | 2.47 | 587 |
| 187 | 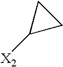 | H | 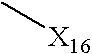 | 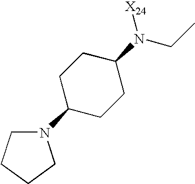 | B | 1.75 | 586 |
| 188 | 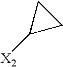 | H | 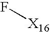 | 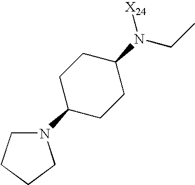 | B | 1.77 | 590 |
| 189 | 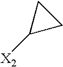 | 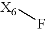 | H | 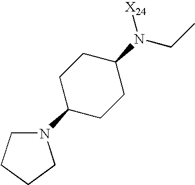 | B | 1.74 | 590 |
| 190 | 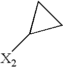 | 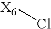 | H | 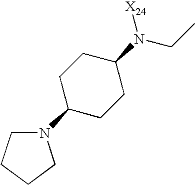 | A | 2.5 | 607 |
| 191 | 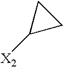 | H | 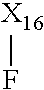 | 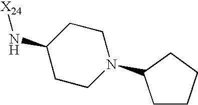 | A | 2.49 | 562 |
| 192 | 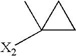 | 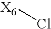 | H | 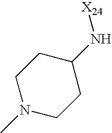 | A | 2.47 | 539 |
| 193 | 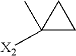 | 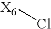 | H | 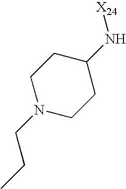 | A | 2.51 | 567 |
| 194 | 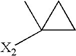 | 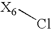 | H | 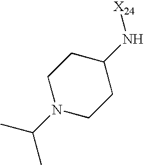 | A | 2.48 | 567 |
| 195 | 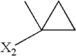 | 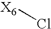 | H | 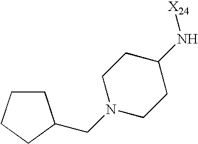 | A | 2.65 | 607 |
| 196 | 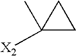 | 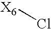 | H | 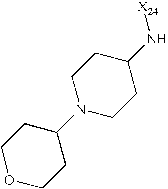 | A | 2.45 | 609 |
| 197 | 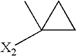 | 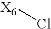 | H | 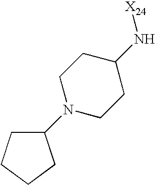 | A | 2.56 | 593 |
| 198 | 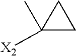 | 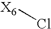 | H | 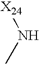 | A | 2.74 | 456 |
| 199 | 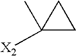 | 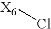 | H | 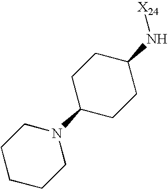 | A | 2.54 | 607 |
| 200 | 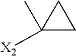 | 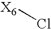 | H | 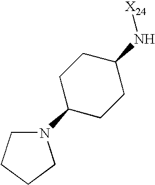 | A | 2.5 | 593 |
| 201 | 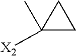 | 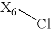 | H | 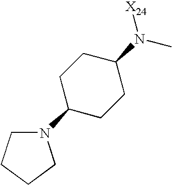 | A | 2.51 | 607 |
| 202 | 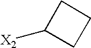 | 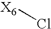 | H | 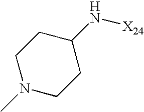 | B | 1.72 | 539 |
| 203 | 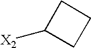 | 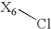 | H | 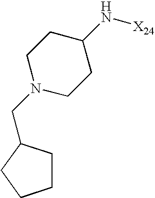 | A | 2.67 | 607 |
| 204 | 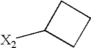 | 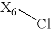 | H | 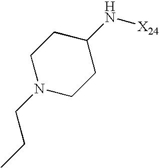 | A | 2.25 | 567 |
| 205 | 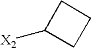 | 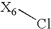 | H | 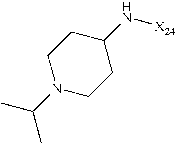 | A | 2.51 | 567 |
| 206 | 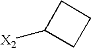 | 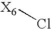 | H | 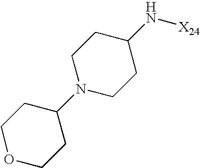 | A | 2.48 | 609 |
| 207 | 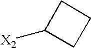 | 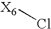 | H | 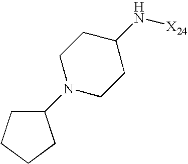 | A | 2.57 | 593 |
| 208 | 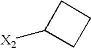 | 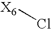 | H | 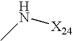 | A | 2.77 | 456 |
| 209 | 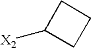 | 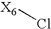 | H | 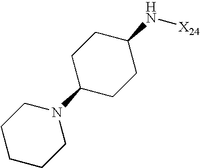 | A | 2.58 | 607 |
| 210 | 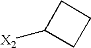 | 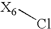 | H | 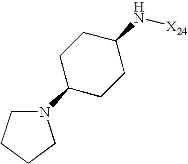 | A | 2.53 | 593 |
| 211 | 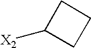 | 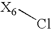 | H | 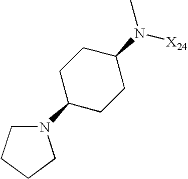 | A | 2.53 | 607 |
| 212 | 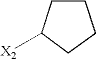 | 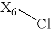 | H | 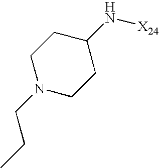 | A | 2.65 | 581 |
| 213 | 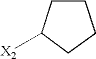 | 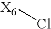 | H | 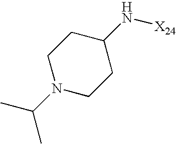 | A | 2.67 | 581 |
| 214 | 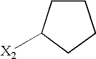 | 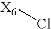 | H | 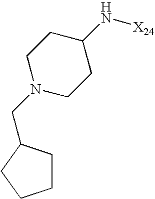 | A | 2.76 | 621 |
| 215 | 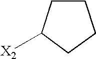 | 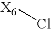 | H | 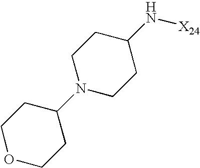 | A | 2.57 | 623 |
| 216 | 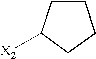 | 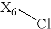 | H | 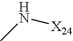 | A | 2.96 | 470 |
| 217 | 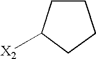 | 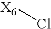 | H | 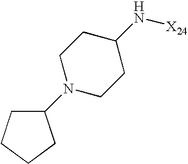 | A | 2.68 | 607 |
| 218 | 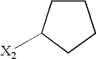 | 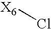 | H | 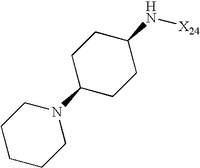 | A | 2.77 | 621 |
| 219 | 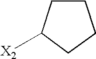 | 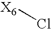 | H | 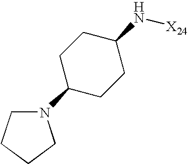 | A | 2.68 | 607 |
| 220 | 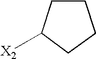 | 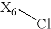 | H | 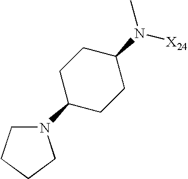 | A | 2.69 | 621 |

### Example 14

### 2-[4-(7-acetylamino-3-cyclopropyl-4,5-dihydro-pyrazolo[3′,4′:3,4]benzo[1,2-d]thiazol-1-yl)-3-chloro-phenyl]-acetamide

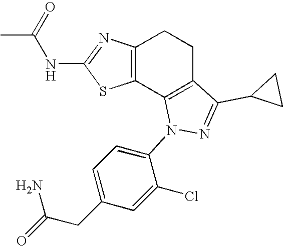

50 mg (0.113 mmol) [4-(7-acetylamino-3-cyclopropyl-4,5-dihydro-pyrazolo[3′,4′:3,4]benzo[1,2-d]thiazol-1-yl)-3-chloro-phenyl]-acetic acid, 55.80 mg (0.147 mmol) HATU and 95.45 μL (0.700 mmol) triethylamine are stirred in 4 mL dichloromethane for 0.5 h at ambient temperature, then 225.78 μL (0.452 mmol) 2 molar ammonia solution in ethanol are added. The reaction mixture is stirred for 16 hours at ambient temperature. Then the mixture is extracted with 5% potassium carbonate solution and dichloromethane, the organic phase is dried and evaporated to dryness. The residue is purified by chromatography. The product is crystallised from ethyl acetate/petroleum ether. Yield: 16.60 mg (33%), HPLC-MS: method A, RT=2.57 min, MH+=441.

The following compounds are prepared analogously:

TABLE D

| | | | | | | (ID) |
|---|---|---|---|---|---|---|
| 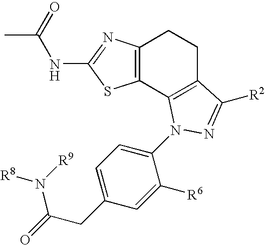 | | | | | | |
| Ex. | | | | meth- | RT | M + |
| D | R² | R⁶ | NR⁸R⁹ (═R²²) | od | [min] | H |
| 1 | 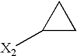 | 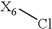 | 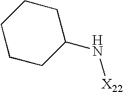 | A | 3.14 | 524 |
| 2 | 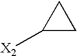 | 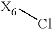 | 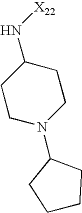 | A | 2.48 | 593 |
| 3 | 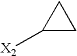 | 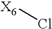 | 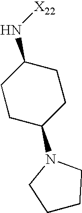 | A | 2.41 | 593 |
| 4 | 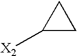 | 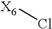 | 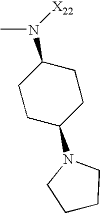 | A | 2.44 | 607 |
| 5 | 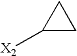 | 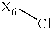 | 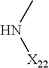 | A | 2.64 | 455 |
| 6 | 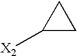 | 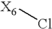 | 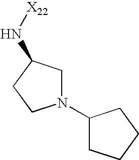 | A | 2.50 | 579 |

### Example 15

### N-[1-(2-chloro-4-hydroxy-phenyl)-3-cyclopropyl-4,5-dihydro-1H-pyrazolo[3′,4′:3,4]benzo[1,2-d]thiazol-7-yl]-acetamide

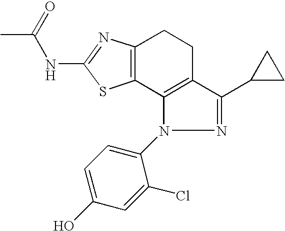

192 mg (0.448 mmol) 4-(7-acetylamino-3-cyclopropyl-4,5-dihydro-pyrazolo[3′,4′:3,4]benzo[1,2-d]thiazol-1-yl)-3-chloro-phenyl-boric acid and 38.51 μL (0.448 mmol) hydrogen peroxide (35%) are stirred in 4 mL water for 16 hours at ambient temperature. Then the product is suction filtered, washed with water and dried.

Yield: 134.7 mg (75%). HPLC-MS: method C, RT=3.58 min, MH+=401.

### Example 16

### trans-4-(7-acetylamino-3-cyclopropyl-4,5-dihydro-pyrazolo[3′,4′:3,4]benzo[1,2-d]thiazol-1-yl)-cyclohexanecarboxylic acid (1-cyclopentyl-piperidin-4-yl)-amide

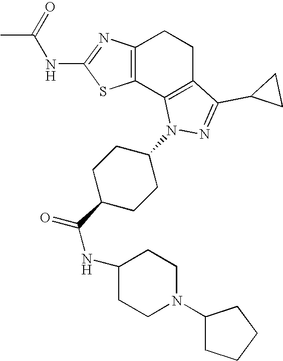

50 mg (0.125 mmol) trans-4-(7-acetylamino-3-cyclopropyl-4,5-dihydro-pyrazolo[3′,4′:3,4]benzo[1,2-d]thiazol-1-yl)-cyclohexanecarboxylic acid are placed in 3 mL dimethylformamide, 60 mg (0.158 mmol) HATU and 100 μL (0.588 mmol) diisopropylethylamine are added. The reaction mixture is stirred for 0.25 h at ambient temperature, then 35 mg (0.145 mmol) 4-amino-1-cyclopentyl-piperidin dihydrochloride are added. The mixture is stirred for 1 hour at ambient temperature gerührt. The suspension is suction filtered, the precipitate is washed with dimethylformamide and water and dried.

Yield: 56 mg (81%). HPLC-MS: method A, RT=2.40 min, MH+=551

### Example 17

### cis-4-(7-acetylamino-3-cyclopropyl-4,5-dihydro-pyrazolo[3′,4′:3,4]benzo[1,2-d]thiazol-1-yl)-cyclohexanecarboxylic acid (1-cyclopentyl-piperidin-4-yl)-amide

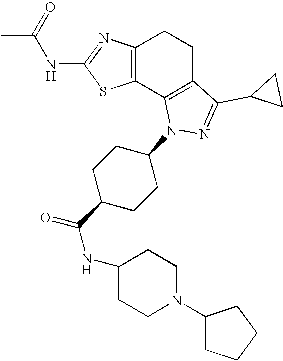

50 mg (0.125 mmol) cis-4-(7-acetylamino-3-cyclopropyl-4,5-dihydro-pyrazolo[3′,4′:3,4]benzo[1,2-d]thiazol-1-yl)-cyclohexanecarboxylic acid are placed in 3 mL dimethylformamide, 60 mg (0.158 mmol) O-(7-azabenzotriazol-1-yl-)-N,N,N′,N′-tetramethyluronium-hexafluoro-phosphate (HATU) and 100 μL (0.588 mmol) diisopropylethylamine are added. The reaction mixture is stirred for 0.25 hours at ambient temperature, then 35 mg (0.145 mmol) 4-amino-1-cyclopentyl-piperidine dihydrochloride are added. The mixture is stirred for 16 hours at ambient temperature. The solution is extracted with dichloromethane and 5% potassium carbonate solution. The organic phase is separated off using a phase separation cartridge and evaporated to dryness. The residue is purified by chromatography (semipreparative RP HPLC). Corresponding fractions are combined and lyophilised. 74 mg (89%). NMR: LG102885. HPLC-MS: method A, RT=2.32 min, MH+=551

The following compounds are prepared analogously:

TABLE E

| | |
|---|---|
| | (IE) |
| 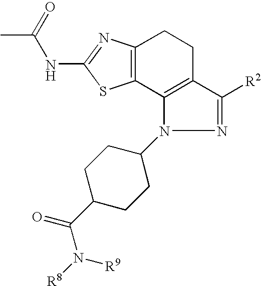 | |

| Ex | | | | | RT | |
|---|---|---|---|---|---|---|
| E | stereo | R² | NR⁸R⁹ (═R²⁴) | method | [min] | M + H |
| 1 | cis | 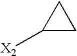 | 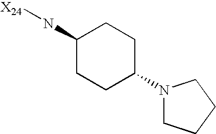 | A | 2.26 | 551 |
| 2 | cis | 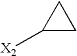 | 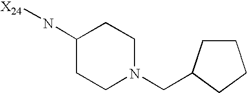 | A | 2.40 | 565 |
| 3 | cis | 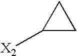 | 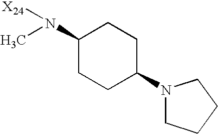 | A | 2.32 | 565 |
| 4 | cis | 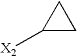 | 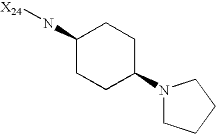 | A | 2.27 | 551 |
| 5 | cis | 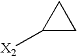 | 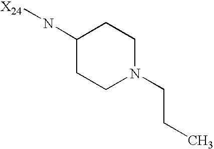 | A | 2.26 | 525 |
| 6 | cis | 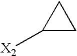 | 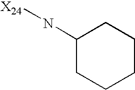 | A | 2.92 | 482 |
| 7 | cis | 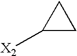 | 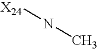 | A | 2.36 | 414 |
| 8 | cis | 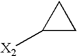 | 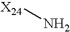 | A | 2.28 | 400 |
| 9 | trans | 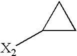 | 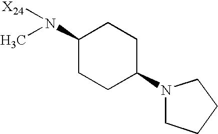 | A | 2.41 | 565 |
| 10 | trans | 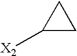 | 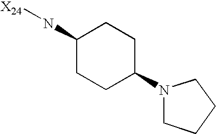 | A | 2.35 | 551 |
| 11 | trans | 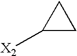 | 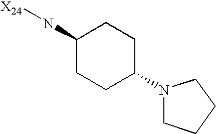 | A | 2.35 | 551 |
| 12 | trans | 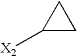 | 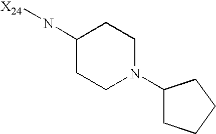 | A | 2.40 | 551 |
| 13 | trans | 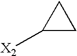 | 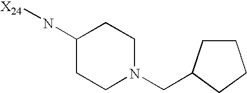 | A | 2.48 | 565 |
| 14 | trans | 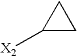 | 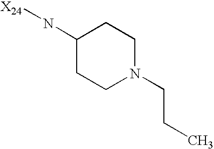 | A | 2.34 | 525 |
| 15 | trans | 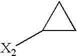 | 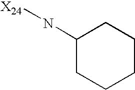 | A | 2.96 | 482 |
| 16 | trans | 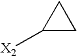 | 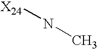 | A | 2.41 | 414 |
| 17 | trans | 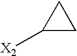 | 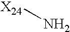 | A | 2.35 | 400 |

### Example 18

### N-{3-cyclopropyl-1-[1-(1-propyl-piperidine-4-carbonyl)-piperidin-4-yl]-4,5-dihydro-1H-pyrazolo[3′,4′:3,4]benzo[1,2-d]thiazol-7-yl}-acetamide

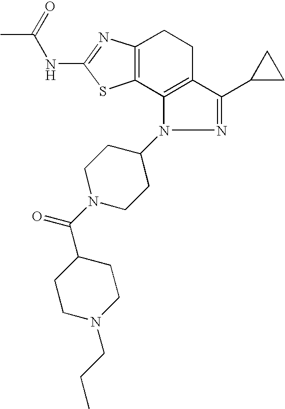

100 mg (0.213 mmol) N-{3-cyclopropyl-1-[1-(piperidine-4-carbonyl)-piperidin-4-yl]-4,5-dihydro-1H-pyrazolo[3′,4′:3,4]benzo[1,2-d]thiazol-7-yl}-acetamide, 20 mg (0.344 mmol) propionaldehyde and 37 mg (0.451 mmol) sodium acetate are stirred in 10 mL dichloromethane/acetonitrile for 24 hours at ambient temperature. Then the mixture is evaporated down, the residue is stirred with 10 mL 5% potassium carbonate solution, suction filtered and washed with water. The precipitate is purified by chromatography (HPLC).

Yield: 43 mg (39%). HPLC-MS: method A, RT=2.34 min, MH+=511/569.

The following compounds are prepared analogously:

TABLE F

| | |
|---|---|
| | (IF) |
| 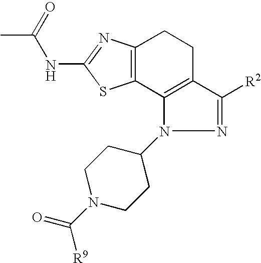 | |

| Ex. | | | | RT | |
|---|---|---|---|---|---|
| F | R² | R⁹ | method | [min] | M + H |
| 1 | 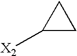 | 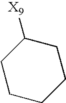 | C | 4.09 | 468 |
| 2 | 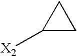 | 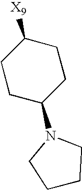 | A | 2.31 | 537 |
| 3 | 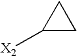 | 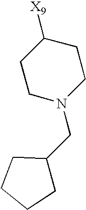 | A | 2.51 | 551 |
| 4 | 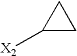 | 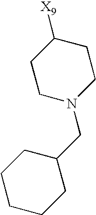 | A | 2.60 | 565 |
| 5 | 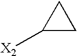 | 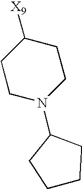 | A | 2.33 | 537 |

### Example 19

### N-{3-cyclopropyl-1-[1-(4-isopropyl-piperazine-1-carbonyl)-piperidin-4-yl]-4,5-dihydro-1H-pyrazolo[3′,4′:3,4]benzo[1,2-d]thiazol-7-yl}-acetamide

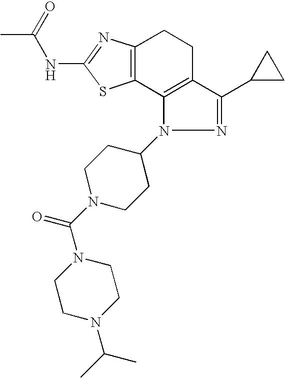

100 mg (0.254 mmol) N-(3-cyclopropyl-1-piperidin-4-yl-4,5-dihydro-1H-pyrazolo[3′,4′:3,4]benzo[1,2-d]thiazol-7-yl)-acetamide, 25 mg (0.084 mmol) triphosgene and 78 μL (0.563 mmol) triethylamine are placed in 20 mL dichloromethane/tetrahydrofuran and refluxed for 1 hour with stirring. Then the mixture is combined with 33 mg (0.257 mmol) isopropyl-piperazine and stirred for 24 hours at ambient temperature. Then it is evaporated down, the residue is stirred with 10 mL of 10% potassium hydrogen carbonate solution and suction filtered. The precipitate is purified by chromatography (HPLC). Yield: 20 mg (15%). HPLC-MS: method A, RT=2.30 min, MH+=512.

The following compounds are prepared analogously:

TABLE G

| | |
|---|---|
| | (IG) |
| 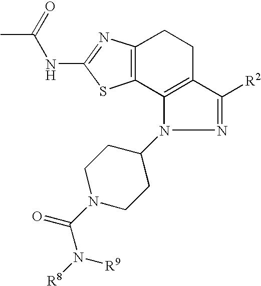 | |

| Ex | | | | RT | |
|---|---|---|---|---|---|
| G | R² | NR⁸R⁹ (═R²²) | method | [min] | M + H |
| 1 | 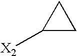 | 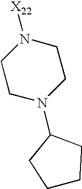 | A | 1.59 | 538 |
| 2 | 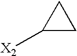 | 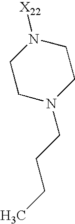 | A | 2.41 | 526 |
| 3 | 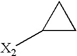 | 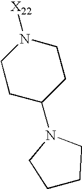 | A | 2.35 | 538 |
| 4 | 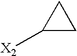 | 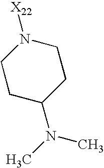 | A | 2.28 | 512 |

Biological Test

The compounds of formula (I) mentioned by way of example are characterised by an affinity for PI3-kinase, i.e. in the test by an IC₅₀ value of below 600 nmol/litre.

In order to determine the inhibitory activity of the compounds on PI3Ky, an in-vitro kinase assay was used. The expression and purification of Gβ₁γ₂-His and p101-GST/p110γ from Sf9-cells (Spodoptera frugiperda 9) has already been described (Maier et al., J. Biol. Chem. 1999 (274) 29311-29317). Alternatively, the following method was used to determine the activity:

10 μl of the compound to be tested were placed on 96 well PVDF filter plates (0.45 μM) and incubated for 20 min with 30 μl lipid vesicles (PIP₂ (0.7 μg/well), phosphatidylethanolamine (7.5 μg/well), phosphatidylserine (7.5 μg/well), sphingomyelin (0.7 μg/well) and phosphatidylcholine (3.2 μg/well)) which contained 1-3 ng PI3K□ and 20-60 ng G□₁□₂-His. The reaction was started by the addition of 10 μl reaction buffer (40 mM Hepes, pH 7.5, 100 mM NaCl, 1 mM EGTA, 1 mM □-glycerophosphate, 1 mM DTT, 7 mM MgCl₂ and 0.1% BSA; 1 μM ATP and 0.2 μCi [□-³³P]-ATP) and incubated for 120 min at ambient temperature. The reaction solution was sucked through the filters by the application of a vacuum and washed with 200 μl PBS. After the plates had been dried at 50° C. the radioactivity remaining in the plates was determined after the addition of 50 μl scintillation liquid using a Top-Count measuring device.

Ranges of Indications

It has been found that the compounds of formula (I) are characterised by a variety of possible applications in the therapeutic field. Particular mention should be made of those applications for which the compounds of formula (I) according to the invention are preferably used by virtue of their pharmaceutical activity as PI3-kinase modulators.

Generally speaking, these are diseases in whose pathology PI3-kinases are implicated, particularly inflammatory and allergic diseases. Particular mention should be made of inflammatory and allergic respiratory complaints, inflammatory diseases of the gastrointestinal tract, inflammatory diseases of the motor apparatus, inflammatory and allergic skin diseases, inflammatory eye diseases, diseases of the nasal mucosa, inflammatory or allergic ailments which involve autoimmune reactions or inflammation of the kidneys. The treatment may be symptomatic, adaptive, curative or preventative.

Respiratory complaints deserving special mention would be chronic and/or obstructive respiratory complaints. The compounds of formula 1 according to the invention may, by virtue of their pharmacological properties, bring about a reduction in
Tissue damage
Inflammation of the airways
bronchial hyperreactivity
the process of reconstruction of the lung as a result of inflammation
worsening of the disease (progression).

The compounds according to the invention are particularly preferred for preparing a medicament for the treatment of chronic bronchitis, acute bronchitis, bronchitis caused by bacterial or viral infection or fungi or helminths, allergic bronchitis, toxic bronchitis, chronic obstructive pulmonary disease (COPD), asthma (intrinsic or allergic), paediatric asthma, bronchiectasis, allergic alveolitis, allergic or non-allergic rhinitis, chronic sinusitis, cystic fibrosis or mucoviscidosis, alpha-1-antitrypsin deficiency, cough, pulmonary emphysema, interstitial lung diseases such as e.g. pulmonary fibrosis, asbestosis and silicosis and alveolitis; hyperreactive airways, nasal polyps, pulmonary oedema such as e.g. toxic pulmonary oedema and ARDS/IRDS, pneumonitis of different origins, e.g. radiation-induced or caused by aspiration or infectious pneumonitis, collagenoses such as lupus erythematodes, systemic sclerodermy, sarcoidosis or Boeck's disease.

The compounds of formula (I) are also suitable for the treatment of diseases of the skin, such as e.g. psoriasis, contact dermatitis, atopic dermatitis, alopecia areata (circular hair loss), erythema exsudativum multiforme (Stevens-Johnson Syndrome), dermatitis herpetiformis, sclerodermy, vitiligo, nettle rash (urticaria), lupus erythematodes, follicular and surface pyodermy, endogenous and exogenous acne, acne rosacea and other inflammatory or allergic or proliferative skin diseases.

Moreover, the compounds of formula (I) are suitable for therapeutic use in cases of inflammatory or allergic complaints which involve autoimmune reactions, such as e.g. inflammatory bowel diseases, e.g. Crohn's disease or ulcerative colitis; diseases of the arthritis type, such as e.g. rheumatoid or psoriatic arthritis, osteoarthritis, rheumatoid spondylitis and other arthritic conditions or multiple sclerosis.

The following general inflammatory or allergic diseases may also be mentioned, which can be treated with medicaments containing compounds of formula (I):
inflammation of the eye, such as e.g. conjunctivitis of various kinds, e.g. caused by infections with fungi or bacteria, allergic conjunctivitis, irritable conjunctivitis, drug-induced conjunctivitis, keratitis, uveitis
diseases of the nasal mucosa, such as e.g. allergic rhinitis/sinusitis or nasal polyps
inflammatory or allergic conditions, such as e.g. systemic lupus erythematodes, chronic hepatitis, kidney inflammations such as glomerulonephritis, interstitial nephritis or idiopathic nephrotic syndrome.

Other diseases which may be treated with a drug containing compounds of formula (I) on the basis of their pharmacological activity include toxic or septic shock syndrome, atherosclerosis, middle ear infections (otitis media), hypertrophy of the heart, cardiac insufficiency, stroke, ischaemic reperfusion injury or neurodegenerative diseases such as Parkinson's disease or Alzheimer's.

Combinations

The compounds of formula (I) may be used on their own or in combination with other active substances of formula (I). If desired the compounds of formula (I) may also be used in combination with W, where W denotes a pharmacologically active substance and (for example) is selected from among the betamimetics, anticholinergics, corticosteroids, PDE4-inhibitors, LTD4-antagonists, EGFR-inhibitors, dopamine agonists, H1-antihistamines, PAF-antagonists and PI3-kinase inhibitors, preferably PI3-\tilde{□}Kinase inhibitors. Moreover, double or triple combinations of W may be combined with the compounds of formula (I). Combinations of W might be, for example:
W denotes a betamimetic, combined with an active substance selected from among the anticholinergics, corticosteroids, PDE4-inhibitors, EGFR-inhibitors and LTD4-antagonists,
W denotes an anticholinergic, combined with an active substance selected from among the betamimetics, corticosteroids, PDE4-inhibitors EGFR-inhibitors and LTD4-antagonists,
W denotes a corticosteroid, combined with an active substance selected from among the PDE4-inhibitors, EGFR-inhibitors and LTD4-antagonists
W denotes a PDE4-inhibitor, combined with an active substance selected from among the EGFR-inhibitors and LTD4-antagonists
W denotes an EGFR-inhibitor, combined with an LTD4-antagonist.

The compounds used as betamimetics are preferably compounds selected from among albuterol, arformoterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmefamol, salmeterol, soterenol, sulphonterol, terbutaline, tiaramide, tolubuterol, zinterol, CHF-1035, HOKU-81, KUL-1248 and
3-(4-{6-[2-hydroxy-2-(4-hydroxy-3-hydroxymethyl-phenyl)-ethylamino]-hexyloxy}-butyl)-benzyl-sulphonamide
5-[2-(5.6-diethyl-indan-2-ylamino)-1-hydroxy-ethyl]-8-hydroxy-1H-quinoline-2-one
4-hydroxy-7-[2-{[2-{[3-(2-phenylethoxy)propyl]sulphonyl}ethyl]-amino}ethyl]-2(3H)-benzothiazolone
1-(2-fluoro-4-hydroxyphenyl)-2-[4-(1-benzimidazolyl)-2-methyl-2-butylamino]ethanol
1-[3-(4-methoxybenzyl-amino)-4-hydroxyphenyl]-2-[4-(1-benzimidazolyl)-2-methyl-2-butylamino]ethanol
1-[2H-5-hydroxy-3-oxo-4H-1,4-benzoxazin-8-yl]-2-[3-(4-N,N-dimethylaminophenyl)-2-methyl-2-propylamino]ethanol
1-[2H-5-hydroxy-3-oxo-4H-1,4-benzoxazin-8-yl]-2-[3-(4-methoxyphenyl)-2-methyl-2-propylamino]ethanol
1-[2H-5-hydroxy-3-oxo-4H-1,4-benzoxazin-8-yl]-2-[3-(4-n-butyloxyphenyl)-2-methyl-2-propylamino]ethanol
1-[2H-5-hydroxy-3-oxo-4H-1,4-benzoxazin-8-yl]-2-{4-[3-(4-methoxyphenyl)-1,2,4-triazole-3-yl]-2-methyl-2-butylamino}ethanol
5-hydroxy-8-(1-hydroxy-2-isopropylaminobutyl)-2H-1,4-benzoxazin-3-(4H)-on
1-(4-amino-3-chloro-5-trifluoromethylphenyl)-2-tert.-butylamino)ethanol
6-hydroxy-8-{1-hydroxy-2-[2-(4-methoxy-phenyl)-1,1-dimethyl-ethylamino]-ethyl}-4H-benzo[1,4]oxazin-3-one
6-hydroxy-8-{1-hydroxy-2-[2-(ethyl 4-phenoxy-acetate)-1,1-dimethyl-ethylamino]-ethyl}-4H-benzo[1,4]oxazin-3-one
6-hydroxy-8-{1-hydroxy-2-[2-(4-phenoxy-acetic acid)-1,1-dimethyl-ethylamino]-ethyl}-4H-benzo[1,4]oxazin-3-one
8-{2-[1,1-dimethyl-2-(2.4.6-trimethylphenyl)-ethylamino]-1-hydroxy-ethyl}-6-hydroxy-4H-benzo[1,4]oxazin-3-one
6-hydroxy-8-{1-hydroxy-2-[2-(4-hydroxy-phenyl)-1,1-dimethyl-ethylamino]-ethyl}-4H-benzo[1,4]oxazin-3-one
6-hydroxy-8-{1-hydroxy-2-[2-(4-isopropyl-phenyl)-1,1-dimethyl-ethylamino]-ethyl}-4H-benzo[1,4]oxazin-3-one
8-{2-[2-(4-ethyl-phenyl)-1,1-dimethyl-ethylamino]-1-hydroxy-ethyl}-6-hydroxy-4H-benzo[1,4]oxazin-3-one
8-{2-[2-(4-ethoxy-phenyl)-1,1-dimethyl-ethylamino]-1-hydroxy-ethyl}-6-hydroxy-4H-benzo[1,4]oxazin-3-one
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid
8-{2-[2-(3,4-difluoro-phenyl)-1,1-dimethyl-ethylamino]-1-hydroxy-ethyl}-6-hydroxy-4H-benzo[1,4]oxazin-3-one
1-(4-ethoxy-carbonylamino-3-cyano-5-fluorophenyl)-2-(tert-butylamino)ethanol
   optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The anticholinergics used are preferably compounds selected from among the tiotropium salts, preferably the bromide salt, oxitropium salts, preferably the bromide salt, flutropium salts, preferably the bromide salt, ipratropium salts, preferably the bromide salt, glycopyrronium salts, preferably the bromide salt, trospium salts, preferably the chloride salt, tolterodine. In the above-mentioned salts the cations are the pharmacologically active constituents. As anions the above-mentioned salts may preferably contain the chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts the chlorides, bromides, iodides and methanesulphonates are particularly preferred.

Other specified compounds are:
tropenol 2,2-diphenylpropionate methobromide
scopine 2,2-diphenylpropionate methobromide
scopine 2-fluoro-2,2-diphenylacetate methobromide
tropenol 2-fluoro-2,2-diphenylacetate methobromide
tropenol 3,3′,4,4′-tetrafluorobenzilate methobromide
scopine 3,3′,4,4′-tetrafluorobenzilate methobromide
tropenol 4,4′-difluorobenzilate methobromide
scopine 4,4′-difluorobenzilate methobromide
tropenol 3,3′-difluorobenzilate methobromide
scopine 3,3′-difluorobenzilate methobromide
tropenol 9-hydroxy-fluorene-9-carboxylate methobromide
tropenol 9-fluoro-fluorene-9-carboxylate methobromide
scopine 9-hydroxy-fluorene-9-carboxylate methobromide
scopine 9-fluoro-fluorene-9-carboxylate methobromide
tropenol 9-methyl-fluorene-9-carboxylate methobromide
scopine 9-methyl-fluorene-9-carboxylate methobromide
cyclopropyltropine benzilate methobromide
cyclopropyltropine 2,2-diphenylpropionate methobromide
cyclopropyltropine 9-hydroxy-xanthene-9-carboxylate methobromide
cyclopropyltropine 9-methyl-fluorene-9-carboxylate methobromide
cyclopropyltropine 9-methyl-xanthene-9-carboxylate methobromide
cyclopropyltropine 9-hydroxy-fluorene-9-carboxylate methobromide
cyclopropyltropine methyl 4,4′-difluorobenzilate methobromide
tropenol 9-hydroxy-xanthene-9-carboxylate methobromide
scopine 9-hydroxy-xanthene-9-carboxylate methobromide
tropenol 9-methyl-xanthene-9-carboxylate-methobromide
scopine 9-methyl-xanthene-9-carboxylate-methobromide
tropenol 9-ethyl-xanthene-9-carboxylate methobromide
tropenol 9-difluoromethyl-xanthene-9-carboxylate methobromide
scopine 9-hydroxymethyl-xanthene-9-carboxylate methobromide

As corticosteroids it is preferable to use compounds selected from among prednisolone, prednisone, butixocort propionate, flunisolide, beclomethasone, triamcinolone, budesonide, fluticasone, mometasone, ciclesonide, rofleponide, dexamethasone, betamethasone, deflazacort, RPR-106541, NS-126, ST-26 and
(S)-fluoromethyl 6,9-difluoro-17-[(2-furanylcarbonyl)oxy]-11-hydroxy-16-methyl-3-oxo-androsta-1,4-diene-17-carbothionate
(S)-(2-oxo-tetrahydro-furan-3S-yl)6,9-difluoro-11-hydroxy-16-methyl-3-oxo-17-propionyloxy-androsta-1,4-diene-17-carbothionate,
etiprednol-dichloroacetate
   optionally in the form of the racemates, enantiomers or diastereomers thereof and optionally in the form of the salts and derivatives thereof, the solvates and/or hydrates thereof. Any reference to steroids includes a reference to any salts or derivatives, hydrates or solvates thereof which may exist. Examples of possible salts and derivatives of the steroids may be: alkali metal salts, such as for example sodium or potassium salts, sulphobenzoates, phosphates, isonicotinates, acetates, propionates, dihydrogen phosphates, palmitates, pivalates or furoates.

PDE4-inhibitors which may be used are preferably compounds selected from among enprofyllin, theophyllin, roflumilast, ariflo (cilomilast), tofimilast, pumafentrin, lirimilast, arofyllin, atizoram, D-4418, Bay-198004, BY343, CP-325.366, D-4396 (Sch-351591), AWD-12-281 (GW-842470), NCS-613, CDP-840, D-4418, PD-168787, T-440, T-2585, V-11294A, CI-1018, CDC-801, CDC-3052, D-22888, YM-58997, Z-15370 and
N-(3,5-dichloro-1-oxo-pyridin-4-yl)-4-difluoromethoxy-3-cyclopropylmethoxybenzamide
(−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide
(R)-(+)-1-(4-bromobenzyl)-4-[(3-cyclopentyloxy)-4-methoxyphenyl]-2-pyrrolidone
3-(cyclopentyloxy-4-methoxyphenyl)-1-(4-N′-[N-2-cyano-S-methyl-isothioureido]benzyl)-2-pyrrolidone
cis[4-cyano-4-(3-cyclopentyloxy-4-methoxyphenyl)cyclohexane-1-carboxylic acid]
2-carbomethoxy-4-cyano-4-(3-cyclopropylmethoxy-4-difluoromethoxy-phenyl)cyclohexan-1-one
cis[4-cyano-4-(3-cyclopropylmethoxy-4-difluoromethoxyphenyl)cyclohexan-1-ol]
(R)-(+)-ethyl[4-(3-cyclopentyloxy-4-methoxyphenyl)pyrrolidin-2-ylidene]acetate
(S)-(−)-ethyl[4-(3-cyclopentyloxy-4-methoxyphenyl)pyrrolidin-2-ylidene]acetate
9-cyclopentyl-5,6-dihydro-7-ethyl-3-(2-thienyl)-9H-pyrazolo[3,4-c]-1,2,4-triazolo[4.3-a]pyridine
9-cyclopentyl-5,6-dihydro-7-ethyl-3-(tert-butyl)-9H-pyrazolo[3,4-c]-1,2,4-triazolo[4.3-a]pyridine
   optionally in the form of the racemates, enantiomers or diastereomers thereof and optionally in the form of the pharmacologically acceptable acid addition salts thereof, the solvates and/or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The LTD4-antagonists used are preferably compounds selected from among montelukast, pranlukast, zafirlukast, MCC-847 (ZD-3523), MN-001, MEN-91507 (LM-1507), VUF-5078, VUF-K-8707, L-733321 and
1-(((R)-(3-(2-(6,7-difluoro-2-quinolinyl)ethenyl)phenyl)-3-(2-(2-hydroxy-2-propyl)phenyl)thio)methylcyclopropane-acetic acid,
1-(((1(R)-3(3-(2-(2,3-dichlorothieno[3,2-b]pyridin-5-yl)-(E)-ethenyl)phenyl)-3-(2-(1-hydroxy-1-methylethyl)phenyl )propyl)thio)methyl)cyclopropaneacetic acid
[2-[[2-(4-tert-butyl-2-thiazolyl)-5-benzofuranyl]oxymethyl]phenyl]acetic acid
   optionally in the form of the racemates, enantiomers or diastereomers thereof and optionally in the form of the pharmacologically acceptable acid addition salts, solvates and/or hydrates thereof. According to the invention the acid addition salts of the betamimetics are preferably selected from among the hydrochloride, hydrobromide, hydroiodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate. By salts or derivatives which the LTD4-antagonists may optionally be capable of forming are meant, for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

EGFR-inhibitors which may be used are preferably compounds selected from among cetuximab, trastuzumab, ABX-EGF, Mab ICR-62 and
4-[(3-chloro-4-fluorophenyl)amino]-6-{[4-(morpholin-4-yl)-1-oxo-2-buten-1-yl]-amino}-7-cyclopropylmethoxy-quinazoline
4-[(3-chloro-4-fluorophenyl)amino]-6-{[4-(N,N-diethylamino)-1-oxo-2-buten-1-yl]amino}-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopropylmethoxy-quinazoline
4-[(R)-(1-phenyl-ethyl)amino]-6-{[4-(morpholin-4-yl)-1-oxo-2-buten-1-yl]amino}-7-cyclopentyloxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline
4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline
4-[(3-chloro-4-fluoro-phenyl)amino]-6-[2-((S)-6-methyl-2-oxo-morpholin-4-yl)-ethoxy]-7-methoxy-quinazoline
4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopentyloxy-quinazoline
4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline
4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline
4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline
4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline
4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline
4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline
4-[(3-chloro-4-fluorophenyl)amino]-6-{[4-(N-cyclopropyl-N-methyl-amino)-1-oxo-2-buten-1-yl]amino}-7-cyclopentyloxy-quinazoline
4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline
4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline
4-[(3-ethynyl-phenyl)amino]-6.7-bis-(2-methoxy-ethoxy)-quinazoline
4-[(3-chloro-4-fluorophenyl)amino]-7-[3-(morpholin-4-yl)-propyloxy]-6-[(vinyl-carbonyl)amino]-quinazoline
4-[(R)-(1-phenyl-ethyl)amino]-6-(4-hydroxy-phenyl)-7H-pyrrolo[2,3-d]pyrimidine
3-cyano-4-[(3-chloro-4-fluorophenyl)amino]-6-{[4-(N,N-dimethylamino)-1-oxo-2-buten-1-yl]amino}-7-ethoxy-quinoline
4-{[3-chloro-4-(3-fluoro-benzyloxy)-phenyl]amino}-6-(5-{[(2-methanesulphonyl-ethyl)amino]methyl}-furan-2-yl)quinazoline
4-[(R)-(1-phenyl-ethyl)amino]-6-{[4-((R)-6-methyl-2-oxo-morpholin-4-yl)-1-oxo-2-buten-1-yl]amino}-7-methoxy-quinazoline
4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazoline
4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline
4-[(3-ethynyl-phenyl)amino]-6-{[4-(5.5-dimethyl-2-oxo-morpholin-4-yl)-1-oxo-2-buten-1-yl]amino}-quinazoline
4-[(3-chloro-4-fluoro-phenyl)amino]-6-[2-(2.2-dimethyl-6-oxo-morpholin-4-yl)-ethoxy]-7-methoxy-quinazoline
4-[(3-chloro-4-fluoro-phenyl)amino]-6-[2-(2.2-dimethyl-6-oxo-morpholin-4-yl)-ethoxy]-7-[(R)-(tetrahydrofuran-2-yl)methoxy]-quinazoline
4-[(3-chloro-4-fluoro-phenyl)amino]-7-[2-(2.2-dimethyl-6-oxo-morpholin-4-yl)-ethoxy]-6-[(S)-(tetrahydrofuran-2-yl)methoxy]-quinazoline
4-[(3-chloro-4-fluoro-phenyl)amino]-6-{2-[4-(2-oxo-morpholin-4-yl)-piperidin-1-yl]-ethoxy}-7-methoxy-quinazoline
4-[(3-chloro-4-fluoro-phenyl)amino]-6-[1-(tert.-butyloxycarbonyl)-piperidin-4-yloxy]-7-methoxy-quinazoline
4-[(3-chloro-4-fluoro-phenyl)amino]-6-(trans-4-amino-cyclohexan-1-yloxy)-7-methoxy-quinazoline
4-[(3-chloro-4-fluoro-phenyl)amino]-6-(trans-4-methanesulphonylamino-cyclohexan-1-yloxy)-7-methoxy-quinazoline
4-[(3-chloro-4-fluoro-phenyl)amino]-6-(tetrahydropyran-3-yloxy)-7-methoxy-quinazoline
4-[(3-chloro-4-fluoro-phenyl)amino]-6-(1-methyl-piperidin-4-yloxy)-7-methoxy-quinazoline
4-[(3-chloro-4-fluoro-phenyl)amino]-6-{1-[(morpholin-4-yl)carbonyl]-piperidin-4-yloxy}-7-methoxy-quinazoline
4-[(3-chloro-4-fluoro-phenyl)amino]-6-{1-[(methoxymethyl)carbonyl]-piperidin-4-yloxy}-7-methoxy-quinazoline
4-[(3-chloro-4-fluoro-phenyl)amino]-6-(piperidin-3-yloxy)-7-methoxy-quinazoline
4-[(3-chloro-4-fluoro-phenyl)amino]-6-[1-(2-acetylamino-ethyl)-piperidin-4-yloxy]-7-methoxy-quinazoline
4-[(3-chloro-4-fluoro-phenyl)amino]-6-(tetrahydropyran-4-yloxy)-7-ethoxy-quinazoline
4-[(3-chloro-4-fluoro-phenyl)amino]-6-((S)-tetrahydrofuran-3-yloxy)-7-hydroxy-quinazoline
4-[(3-chloro-4-fluoro-phenyl)amino]-6-(tetrahydropyran-4-yloxy)-7-(2-methoxy-ethoxy)-quinazoline
4-[(3-chloro-4-fluoro-phenyl)amino]-6-{trans-4-[(dimethylamino)sulphonylamino]-cyclohexan-1-yloxy}-7-methoxy-quinazoline
4-[(3-chloro-4-fluoro-phenyl)amino]-6-{trans-4-[(morpholin-4-yl)carbonylamino]-cyclohexan-1-yloxy}-7-methoxy-quinazoline
4-[(3-chloro-4-fluoro-phenyl)amino]-6-{trans-4-[(morpholin-4-yl)sulphonylamino]-cyclohexan-1-yloxy}-7-methoxy-quinazoline
4-[(3-chloro-4-fluoro-phenyl)amino]-6-(tetrahydropyran-4-yloxy)-7-(2-acetylamino-ethoxy)-quinazoline
4-[(3-chloro-4-fluoro-phenyl)amino]-6-(tetrahydropyran-4-yloxy)-7-(2-methanesulphonylamino-ethoxy)-quinazoline
4-[(3-chloro-4-fluoro-phenyl)amino]-6-{1-[(piperidin-1-yl)carbonyl]-piperidin-4-yloxy}-7-methoxy-quinazoline
4-[(3-chloro-4-fluoro-phenyl)amino]-6-(1-aminocarbonylmethyl-piperidin-4-yloxy)-7-methoxy-quinazoline
4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline
4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline
4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline
4-[(3-chloro-4-fluoro-phenyl)amino]-6-(trans-4-ethansulphonylamino-cyclohexan-1-yloxy)-7-methoxy-quinazoline
4-[(3-chloro-4-fluoro-phenyl)amino]-6-(1-methanesulphonyl-piperidin-4-yloxy)-7-ethoxy-quinazoline
4-[(3-chloro-4-fluoro-phenyl)amino]-6-(1-methanesulphonyl-piperidin-4-yloxy)-7-(2-methoxy-ethoxy)-quinazoline
4-[(3-chloro-4-fluoro-phenyl)amino]-6-[1-(2-methoxy-acetyl)-piperidin-4-yloxy]-7-(2-methoxy-ethoxy)-quinazoline
4-[(3-chloro-4-fluoro-phenyl)amino]-6-(cis-4-acetylamino-cyclohexan-1-yloxy)-7-methoxy-quinazoline
4-[(3-ethynyl-phenyl)amino]-6-[1-(tert.-butyloxycarbonyl)-piperidin-4-yloxy]-7-methoxy-quinazoline
4-[(3-ethynyl-phenyl)amino]-6-[tetrahydropyran-4-yloxy]-7-methoxy-quinazoline
4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline
4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline
4-[(3-chloro-4-fluoro-phenyl)amino]-6-{cis-4-[(morpholin-4-yl)carbonylamino]-cyclohexan-1-yloxy}-7-methoxy-quinazoline
4-[(3-chloro-4-fluoro-phenyl)amino]-6-{1-[2-(2-oxopyrrolidin-1-yl)ethyl]-piperidin-4-yloxy}-7-methoxy-quinazoline
4-[(3-chloro-4-fluoro-phenyl)amino]-6-{1-[(morpholin-4-yl)carbonyl]-piperidin-4-yloxy}-7-(2-methoxy-ethoxy)-quinazoline
4-[(3-ethynyl-phenyl)amino]-6-(1-acetyl-piperidin-4-yloxy)-7-methoxy-quinazoline
4-[(3-ethynyl-phenyl)amino]-6-(1-methyl-piperidin-4-yloxy)-7-methoxy-quinazoline
4-[(3-ethynyl-phenyl)amino]-6-(1-methanesulphonyl-piperidin-4-yloxy)-7-methoxy-quinazoline
4-[(3-chloro-4-fluoro-phenyl)amino]-6-(1-methyl-piperidin-4-yloxy)-7(2-methoxy-ethoxy)-quinazoline
4-[(3-chloro-4-fluoro-phenyl)amino]-6-(1-isopropyloxycarbonyl-piperidin-4-yloxy)-7-methoxy-quinazoline
4-[(3-chloro-4-fluoro-phenyl)amino]-6-(cis-4-methylamino-cyclohexan-1-yloxy)-7-methoxy-quinazoline
4-[(3-chloro-4-fluoro-phenyl)amino]-6-{cis-4-[N-(2-methoxy-acetyl)-N-methyl-amino]-cyclohexan-1-yloxy}-7-methoxy-quinazoline
4-[(3-ethynyl-phenyl)amino]-6-(piperidin-4-yloxy)-7-methoxy-quinazoline
4-[(3-ethynyl-phenyl)amino]-6-[1-(2-methoxy-acetyl)-piperidin-4-yloxy]-7-methoxy-quinazoline
4-[(3-ethynyl-phenyl)amino]-6-{1-[(morpholin-4-yl)carbonyl]-piperidin-4-yloxy}-7-methoxy-quinazoline
4-[(3-chloro-4-fluoro-phenyl)amino]-6-{1-[(cis-2,6-dimethyl-morpholin-4-yl)carbonyl]-piperidin-4-yloxy}-7-methoxy-quinazoline
4-[(3-chloro-4-fluoro-phenyl)amino]-6-{1-[(2-methyl-morpholin-4-yl)carbonyl]-piperidin-4-yloxy}-7-methoxy-quinazoline
4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline
4-[(3-chloro-4-fluoro-phenyl)amino]-6-{1-[(N-methyl-N-2-methoxyethyl-amino)carbonyl]-piperidin-4-yloxy}-7-methoxy-quinazoline
4-[(3-chloro-4-fluoro-phenyl)amino]-6-(1-ethyl-piperidin-4-yloxy)-7-methoxy-quinazoline
4-[(3-chloro-4-fluoro-phenyl)amino]-6-{1-[(2-methoxyethyl)carbonyl]-piperidin-4-yloxy}-7-methoxy-quinazoline
4-[(3-chloro-4-fluoro-phenyl)amino]-6-{1-[(3-methoxypropyl-amino)-carbonyl]-piperidin-4-yloxy}-7-methoxy-quinazoline
4-[(3-chloro-4-fluoro-phenyl)amino]-6-[cis-4-(N-methanesulphonyl-N-methyl-amino)-cyclohexan-1-yloxy]-7-methoxy-quinazoline
4-[(3-chloro-4-fluoro-phenyl)amino]-6-[cis-4-(N-acetyl-N-methyl-amino)-cyclohexan-1-yloxy]-7-methoxy-quinazoline
4-[(3-chloro-4-fluoro-phenyl)amino]-6-(trans-4-methylamino-cyclohexan-1-yloxy)-7-methoxy-quinazoline
4-[(3-chloro-4-fluoro-phenyl)amino]-6-[trans-4-(N-methanesulphonyl-N-methyl-amino)-cyclohexan-1-yloxy]-7-methoxy-quinazoline
4-[(3-chloro-4-fluoro-phenyl)amino]-6-(trans-4-dimethylamino-cyclohexan-1-yloxy)-7-methoxy-quinazoline
4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline
4-[(3-chloro-4-fluoro-phenyl)amino]-6-[2-(2.2-dimethyl-6-oxo-morpholin-4-yl)-ethoxy]-7-[(S)-(tetrahydrofuran-2-yl)methoxy]-quinazoline
4-[(3-chloro-4-fluoro-phenyl)amino]-6-(1-methanesulphonyl-piperidin-4-yloxy)-7-methoxy-quinazoline
4-[(3-chloro-4-fluoro-phenyl)amino]-6-(1-cyano-piperidin-4-yloxy)-7-methoxy-quinazoline
   optionally in the form of the racemates, enantiomers, diastereomers thereof and optionally in the form of the pharmacologically acceptable acid addition salts, solvates or hydrates thereof. According to the invention the preferred acid addition salts of the betamimetics are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The dopamine agonists used are preferably compounds selected from among bromocriptin, cabergoline, alpha-dihydroergocryptine, lisuride, pergolide, pramipexol, roxindol, ropinirol, talipexol, tergurid and viozan, optionally in the form of the racemates, enantiomers, diastereomers thereof and optionally in the form of the pharmacologically acceptable acid addition salts, solvates or hydrates thereof. According to the invention the preferred acid addition salts of the betamimetics are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydrooxalate, hydrosuccinate, hydrobenzoate and hydro-p-toluenesulphonate.

H1-Antihistamines which may be used are preferably compounds selected from among epinastine, cetirizine, azelastine, fexofenadine, levocabastine, loratadine, mizolastine, ketotifen, emedastine, dimetindene, clemastine, bamipine, cexchlorpheniramine, pheniramine, doxylamine, chlorophenoxamine, dimenhydrinate, diphenhydramine, promethazine, ebastine, desloratidine and meclozine, optionally in the form of the racemates, enantiomers, diastereomers thereof and optionally in the form of the pharmacologically acceptable acid addition salts, solvates or hydrates thereof. According to the invention the preferred acid addition salts of the betamimetics are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The PAF-antagonists used are preferably compounds selected from among
4-(2-chlorophenyl)-9-methyl-2-[3(4-morpholinyl)-3-propanon-1-yl]-6H-thieno-[3,2-f]-[1,2,4]triazolo[4,3-a][1,4]diazepine
6-(2-chlorophenyl)-8,9-dihydro-1-methyl-8-[(4-morpholinyl)carbonyl]-4H,7H-cyclo-penta-[4,5]thieno-[3,2-f][1,2,4]triazolo[4,3-a][1,4]diazepine,
   optionally in the form of the racemates, enantiomers, diastereomers thereof and optionally in the form of the pharmacologically acceptable acid addition salts, solvates or hydrates thereof. According to the invention the preferred acid addition salts of the betamimetics are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The PI3-kinase-δ-inhibitors used are preferably compounds selected from among: IC87114, 2-(6-aminopurin-9-ylmethyl)-3-(2-chlorophenyl)-6.7-dimethoxy-3H-quinazolin-4-one; 2-(6-aminopurin-o-ylmethyl)-6-bromo-3-(2-chlorophenyl )-3H-quinazolin-4-one; 2-(6-aminopurin-o-ylmethyl)-3-(2-chlorophenyl)-7-fluoro-3H-quinazolin-4-one; 2-(6-aminopurin-9-ylmethyl)-6-chloro-3-(2-chlorophenyl)-3H-quinazolin-4-one; 2-(6-aminopurin-9-ylmethyl)-3-(2-chlorophenyl)-5-fluoro-3H-quinazolin-4-one; 2-(6-aminopurin-o-ylmethyl)-5-chloro-3-(2-chloro-phenyl)-3H-quinazolin-4-one; 2-(6-aminopurin-9-ylmethyl)-3-(2-chlorophenyl)-5-methyl-3H-quinazolin-4-one; 2-(6-aminopurin-9-ylmethyl)-8-chloro-3-(2-chlorophenyl)-3H-quinazolin-4-one; 2-(6-aminopurin-9-ylmethyl)-3-biphenyl-2-yl-5-chloro-3H-quinazolin-4-one; 5-chloro-2-(9H-purin-6-ylsulphanylmethyl)-3-o-tolyl-3H-quinazolin-4-one; 5-chloro-3-(2-fluorophenyl)-2-(9H-purin-6-yl-sulphanylmethyl)-3H-quinazolin-4-one; 2-(6-aminopurin-9-ylmethyl)-5-chloro-3-(2-fluorophenyl)-3 H-quinazolin-4-one; 3-biphenyl-2-yl-5-chloro-2-(9H-purin-6-ylsulphanylmethyl)-3H-quinazolin-4-one; 5-chloro-3-(2-methoxyphenyl)-2-(9H-purin-6-yl-sulphanylmethyl)-3H-quinazolin-4-one; 3-(2-chlorophenyl)-5-fluoro-2-(9H-purin-6-yl-sulphanylmethyl)-3H-quinazolin-4-one; 3-(2-chlorophenyl)-6.7-dimethoxy-2-(9H-purin-6-yl-sulphanylmethyl)-3H-quinazolin-4-one; 6-bromo-3-(2-chlorophenyl)-2-(9H-purin-6-yl-sulphanylmethyl)-3H-quinazolin-4-one; 3-(2-chlorophenyl)-8-trifluoromethyl-2-(9H-purin-6-ylsulphanylmethyl)-3H-quinazolin-4-one; 3-(2-chlorophenyl)-2-(9H-purin-6-ylsulphanylmethyl)-3H-benzo[g]quinazolin-4-one; 6-chloro-3-(2-chlorophenyl)-2-(9H-purin-6-yl-sulphanylmethyl)-3H-quinazolin-4-one; 8-chloro-3-(2-chlorophenyl)-2-(9H-purin-6-yl-sulphanylmethyl)-3H-quinazolin-4-one; 3-(2-chlorophenyl)-7-fluoro-2-(9H-purin-6-yl-sulphanylmethyl)-3H-quinazolin-4-one; 3-(2-chlorophenyl)-7-nitro-2-(9H-purin-6-yl-sulphanylmethyl)-3H-quinazolin-4-one; 3-(2-chlorophenyl)-6-hydroxy-2-(9H-purin-6-yl-sulphanylmethyl)-3H-quinazolin-4-one; 5-chloro-3-(2-chlorophenyl)-2-(9H-purin-6-yl-sulphanylmethyl)-3H-quinazolin-4-one; 3-(2-chlorophenyl)-5-methyl-2-(9H-purin-6-yl-sulphanylmethyl)-3H-quinazolin-4-one; 3-(2-chlorophenyl)-6.7-difluoro-2-(9H-purin-6-yl-sulphanylmethyl)-3H-quinazolin-4-one; 3-(2-chlorophenyl)-6-fluoro-2-(9H-purin-6-yl-sulphanylmethyl)-3H-quinazolin-4-one; 2-(6-aminopurin-9-ylmethyl)-3-(2-isopropylphenyl)-5-methyl-3H-quinazolin-4-one; 2-(6-aminopurin-9-ylmethyl)-5-methyl-3-o-tolyl-3H-quinazolin-4-one; 3-(2-fluorophenyl)-5-methyl-2-(9H-purin-6-yl-sulphanylmethyl)-3H-quinazolin-4-one; 2-(6-aminopurin-9-ylmethyl)-5-chloro-3-o-tolyl-3H-quinazolin-4-one; 2-(6-aminopurin-9-ylmethyl)-5-chloro-3-(2-methoxy-phenyl)-3H-quinazolin-4-one; 2-(2-amino-9H-purin-6-ylsulphanylmethyl)-3-cyclopropyl-5-methyl-3H-quinazolin-4-one; 3-cyclopropylmethyl-5-methyl-2-(9H-purin-6-ylsulphanylmethyl)-3H-quinazolin-4-one; 2-(6-aminopurin-9-ylmethyl)-3-cyclopropylmethyl-5-methyl-3H-quinazolin-4-one; 2-(2-amino-9H-purin-6-ylsulphanylmethyl)-3-cyclopropylmethyl-5-methyl-3H-quinazolin-4-one; 5-methyl-3-phenethyl-2-(9H-purin-6-ylsulphanylmethyl)-3H-quinazolin-4-one; 2-(2-amino-9H-purin-6-ylsulphanylmethyl)-5-methyl-3-phenethyl-3H-quinazolin-4-one; 3-cyclopentyl-5-methyl-2-(9H-purin-6-ylsulphanylmethyl)-3H-quinazolin-4-one; 2-(6-aminopurin-9-ylmethyl)-3-cyclopentyl-5-methyl-3H-quinazolin-4-one; 3-(2-chloropyridin-3-yl)-5-methyl-2-(9H-purin-6-ylsulphanylmethyl)-3H-quinazolin-4-one; 2-(6-aminopurin-9-ylmethyl)-3-(2-chloropyridin-3-yl)-5-methyl-3H-quinazolin-4-one; 3-methyl-4-[5-methyl-4-oxo-2-(9H-purin-6-ylsulphanylmethyl)-4H-quinazolin-3-yl]-benzoic acid; 3-cyclopropyl-5-methyl-2-(9H-purin-6-ylsulphanylmethyl)-3H-quinazolin-4-one; 2-(6-aminopurin-9-ylmethyl)-3-cyclopropyl-5-methyl-3H-quinazolin-4-one; 5-methyl-3-(4-nitrobenzyl)-2-(9H-purin-6-ylsulphanylmethyl)-3H-quinazolin-4-one; 3-cyclohexyl-5-methyl-2-(9H-purin-6-ylsulphanylmethyl)-3H-quinazolin-4-one; 2-(6-aminopurin-9-ylmethyl)-3-cyclohexyl-5-methyl-3H-quinazolin-4-one; 2-(2-amino-9H-purin-6-ylsulphanylmethyl)-3-cyclo-hexyl-5-methyl-3H-quinazolin-4-one; 5-methyl-3-(E-2-phenylcyclopropyl)-2-(9H-purin-6-ylsulphanylmethyl)-3H-quinazolin-4-one; 3-(2-chlorophenyl)-5-fluoro-2-[(9H-purin-6-ylamino)methyl]-3H-quinazolin-4-one; 2-[(2-amino-9H-purin-6-ylamino)methyl]-3-(2-chlorophenyl)-5-fluoro-3H-quinazolin-4-one; 5-methyl-2-[(9H-purin-6-ylamino)methyl]-3-o-tolyl-3H-quinazolin-4-one; 2-[(2-amino-9H-purin-6-ylamino)methyl]-5-methyl-3-o-tolyl-3H-quinazolin-4-one; 2-[(2-fluoro-9H-purin-6-ylamino)methyl]-5-methyl-3-o-tolyl-3H-quinazolin-4-one; (2-chlorophenyl)-dimethylamino-(9H-purin-6-ylsulphanylmethyl)-3H-quinazolin-4-one; 5-(2-benzyloxyethoxy)-3-(2-chlorophenyl)-2-(9H-purin-6-ylsulphanylmethyl)-3H-quinazolin-4-one; 3-(2-chlorophenyl)-5-fluoro-4-oxo-3,4-dihydro-quinazolin-2-ylmethyl 6-aminopurine-9-carboxylate; N-[3-(2-chlorophenyl)-5-fluoro-4-oxo-3,4-dihydro-quinazolin-2-ylmethyl]-2-(9H-purin-6-ylsulphanyl)-acetamide; 2-[1-(2-fluoro-9H-purin-6-ylamino)ethyl]-5-methyl-3-o-tolyl-3H-quinazolin-4-one; 5-methyl-2-[1-(9H-purin-6-ylamino)ethyl]-3-o-tolyl-3H-quinazolin-4-one; 2-(6-dimethylaminopurin-9-ylmethyl)-5-methyl-3-o-tolyl-3H-quinazolin-4-one; 5-methyl-2-(2-methyl-6-oxo-1.6-dihydro-purin-7-ylmethyl)-3-o-tolyl-3H-quinazolin-4-one; 5-methyl-2-(2-methyl-6-oxo-1.6-dihydro-purin-9-ylmethyl)-3-o-tolyl-3H-quinazolin-4-one; 2-(amino-dimethylaminopurin-9-ylmethyl)-5-methyl-3-o-tolyl-3H-quinazolin-4-one; 2-(2-amino-9H-purin-6-ylsulphanylmethyl)-5-methyl-3-o-tolyl-3H-quinazolin-4-one; 2-(4-amino-1,3,5-triazin-2-ylsulphanylmethyl)-5-methyl-3-o-tolyl-3H-quinazolin-4-one; 5-methyl-2-(7-methyl-7H-purin-6-ylsulphanylmethyl)-3-o-tolyl-3 H-quinazolin-4-one; 5-methyl-2-(2-oxo-1,2-dihydro-pyrimidin-4-ylsulphanylmethyl)-3-o-tolyl-3H-quinazolin-4-one; 5-methyl-2-purin-7-ylmethyl-3-o-tolyl-3H-quinazolin-4-one; 5-methyl-2-purin-9-ylmethyl-3-o-tolyl-3H-quinazolin-4-one; 5-methyl-2-(9-methyl-9H-purin-6-ylsulphanylmethyl)-3-o-tolyl-3H-quinazolin-4-one; 2-(2,6-diamino-pyrimidin-4-ylsulphanylmethyl)-5-methyl-3-o-tolyl-3H-quinazolin-4-one; 5-methyl-2-(5-methyl-[1,2,4]triazolo[1.5-a]pyrimidin-7-ylsulphanylmethyl)-3-o-tolyl-3H-quinazolin-4-one; 5-methyl-2-(2-methylsulphanyl-9H-purin-6-ylsulphanylmethyl)-3-o-tolyl-3H-quinazolin-4-one; 2-(2-hydroxy-9H-purin-6-ylsulphanylmethyl)-5-methyl-3-o-tolyl-3H-quinazolin-4-one; 5-methyl-2-(1-methyl-1H-imidazol-2-ylsulphanylmethyl)-3-o-tolyl-3H-quinazolin-4-one; 5-methyl-3-o-tolyl-2-(H-[1,2,4]triazol-3-ylsulphanylmethyl)-3H-quinazolin-4-one; 2-(2-amino-6-chloro-purin-9-ylmethyl)-5-methyl-3-o-tolyl-3H-quinazolin-4-one; 2-(6-aminopurin-7-ylmethyl)-5-methyl-3-o-tolyl-3H-quinazolin-4-one; 2-(7-amino-1,2,3-triazolo[4,5-d]pyrimidin-3-yl-methyl)-5-methyl-3-o-tolyl-3H-quinazolin-4-one; 2-(7-amino-1,2,3-triazolo[4,5-d]pyrimidin-1-yl-methyl)-5-methyl-3-o-tolyl-3H-quinazolin-4-one; 2-(6-amino-9H-purin-2-ylsulphanylmethyl)-5-methyl-3-o-tolyl-3H-quinazolin-4-one; 2-(2-amino-6-ethylamino-pyrimidin-4-ylsulphanylmethyl)-5-methyl-3-o-tolyl-3H-quinazolin-4-one; 2-(3-amino-5-methylsulphanyl-1,2,4-triazol-1-yl-methyl)-5-methyl-3-o-tolyl-3H-quinazolin-4-one; 2-(5-amino-3-methylsulphanyl-1,2,4-triazol-1-ylmethyl)-5-methyl-3-o-tolyl-3H-quinazolin-4-one; 5-methyl-2-(6-methylaminopurin-9-ylmethyl)-3-o-tolyl-3H-quinazolin-4-one; 2-(6-benzylaminopurin-9-ylmethyl)-5-methyl-3-o-tolyl-3H-quinazolin-4-one; 2-(2,6-diaminopurin-9-ylmethyl)-5-methyl-3-o-tolyl-3H-quinazolin-4-one; 5-methyl-2-(9H-purin-6-ylsulphanylmethyl)-3-o-tolyl-3H-quinazolin-4-one; 3-isobutyl-5-methyl-2-(9H-purin-6-ylsulphanylmethyl)-3H-quinazolin-4-one; N-{2-[5-methyl-4-oxo-2-(9H-purin-6-ylsulphanylmethyl)-4H-quinazolin-3-yl]-phenyl}-acetamide; 5-methyl-3-(E-2-methyl-cyclohexyl)-2-(9H-purin-6-ylsulphanylmethyl)-3H-quinazolin-4-one; 2-[5-methyl-4-oxo-2-(9H-purin-6-ylsulphanylmethyl)-4H-quinazolin-3-yl]-benzoic acid; 3-{2-[(2-dimethylaminoethyl)methylamino]phenyl}-5-methyl-2-(9H-purin-6-yl-sulphanylmethyl)-3H-quinazolin-4-one; 3-(2-chlorophenyl)-5-methoxy-2-(9H-purin-6-ylsulphanylmethyl)-3H-quinazolin-4-one; 3-(2-chlorophenyl)-5-(2-morpholin-4-yl-ethylamino)-2-(9H-purin-6-ylsulphanylmethyl)-3H-quinazolin-4-one; 3-benzyl-5-methoxy-2-(9H-purin-6-ylsulphanylmethyl)-3H-quinazolin-4-one; 2-(6-aminopurin-9-ylmethyl)-3-(2-benzyloxyphenyl)-5-methyl-3H-quinazolin-4-one; 2-(6-aminopurin-9-ylmethyl)-3-(2-hydroxyphenyl)-5-methyl-3H-quinazolin-4-one; 2-(1-(2-amino-9H-purin-6-ylamino)ethyl)-5-methyl-3-o-tolyl-3H-quinazolin-4-one; 5-methyl-2-[1-(9H-purin-6-ylamino)propyl]-3-o-tolyl-3H-quinazolin-4-one; 2-(1-(2-fluoro-9H-purin-6-ylamino)propyl)-5-methyl-3-o-tolyl-3H-quinazolin-4-one; 2-(1-(2-amino-9H-purin-6-ylamino)propyl)-5-methyl-3-o-tolyl-3H-quinazolin-4-one; 2-(2-benzyloxy-1-(9H-purin-6-ylamino)ethyl)-5-methyl-3-o-tolyl-3H-quinazolin-4-one; 2-(6-aminopurin-9-ylmethyl)-5-methyl-3-{2-(2-(1-methylpyrrolidin-2-yl)-ethoxy)-phenyl}-3H-quinazolin-4-one; 2-(6-aminopurin-9-ylmethyl)-3-(2-(3-dimethylamino-propoxy)-phenyl)-5-methyl-3H-quinazolin-4-one; 2-(6-aminopurin-9-ylmethyl)-5-methyl-3-(2-prop-2-ynyloxyphenyl)-3H-quinazolin-4-one; 2-{2-[1-(6-aminopurin-9-ylmethyl)-5-methyl-4-oxo-4H-quinazolin-3-yl]-phenoxy}-acetamide; 5-chloro-3-(3,5-difluoro-phenyl)-2-[1-(9H-purin-6-ylamino)-propyl]-3H-quinazolin-4-one; 3-phenyl-2-[1-(9H-purin-6-ylamino)-propyl]-3H-quinazolin-4-one; 5-fluoro-3-phenyl-2-[1-(9H-purin-6-ylamino)-propyl]-3 H-quinazolin-4-one; 3-(2,6-difluoro-phenyl)-5-methyl-2-[1-(9H-purin-6-ylamino)-propyl]-3H-quinazolin-4-one; 6-fluoro-3-phenyl-2-[1-(9H-purin-6-ylamino)-ethyl]-3H-quinazolin-4-one; 3-(3,5-difluoro-phenyl)-5-methyl-2-[1-(9H-purin-6-ylamino)-ethyl]-3H-quinazolin-4-one; 5-fluoro-3-phenyl-2-[1-(9H-purin-6-ylamino)-ethyl]-3H-quinazolin-4-one; 3-(2,3-difluoro-phenyl)-5-methyl-2-[1-(9H-purin-6-ylamino)-ethyl]-3H-quinazolin-4-one; 5-methyl-3-phenyl-2-[1-(9H-purin-6-ylamino)-ethyl]-3H-quinazolin-4-one; 3-(3-chloro-phenyl)-5-methyl-2-[1-(9H-purin-6-ylamino)-ethyl]-3H-quinazolin-4-one; 5-methyl-3-phenyl-2-[(9H-purin-6-ylamino)-methyl]-3H-quinazolin-4-one; 2-[(2-amino-9H-purin-6-ylamino)-methyl]-3-(3,5-difluoro-phenyl)-5-methyl-3H-quinazolin-4-one; 3-{2-[(2-diethylamino-ethyl)-methyl-amino]-phenyl}-5-methyl-2-[(9H-purin-6-ylamino)-methyl]-3H-quinazolin-4-one; 5-chloro-3-(2-fluoro-phenyl)-2-[(9H-purin-6-ylamino)-methyl]-3H-quinazolin-4-one; 5-chloro-2-[(9H-purin-6-ylamino)-methyl]-3-o-tolyl-3H-quinazolin-4-one; 5-chloro-3-(2-chloro-phenyl)-2-[(9H-purin-6-ylamino)-methyl]-3H-quinazolin-4-one; 6-fluoro-3-(3-fluoro-phenyl)-2-[1-(9H-purin-6-ylamino)-ethyl]-3H-quinazolin-4-one; 2-[1-(2-amino-9H-purin-6-ylamino)-ethyl]-5-chloro-3-(3-fluoro-phenyl)-3H-quinazolin-4-one; and the pharmaceutically acceptable salts and solvates thereof.

Formulations

The compounds according to the invention may be administered by oral, transdermal, inhalative, parenteral or sublingual route. The compounds according to the invention are present as active ingredients in conventional preparations, for example in compositions consisting essentially of an inert pharmaceutical carrier and an effective dose of the active substance, such as for example tablets, coated tablets, capsules, lozenges, powders, solutions, suspensions, emulsions, syrups, suppositories, transdermal systems etc. An effective dose of the compounds according to the invention is between 0.1 and 5000, preferably between 1 and 500, more preferably between 5-300 mg/dose for oral administration, and between 0.001 and 50, preferably between 0.1 and 10 mg/dose for intravenous, subcutaneous or intramuscular administration. Examples of inhalable formulations include inhalable powders, propellant-containing metered-dose aerosols or propellant-free inhalable solutions. Within the scope of the present invention the term propellant-free inhalable solutions also includes concentrates or sterile ready-to-use inhalable solutions. For use by inhalation it is preferable to use powders, ethanolic or aqueous solutions. For inhalation, according to the invention, solutions containing 0.01 to 1.0, preferably 0.1 to 0.5% active substance are suitable. It is also possible to use the compounds according to the invention as a solution for infusion, preferably in a physiological saline or nutrient saline solution.

The compounds according to the invention may be used on their own or in conjunction with other active substances according to the invention, optionally also in conjunction with other pharmacologically active substances. Suitable formulations include, for example, tablets, capsules, suppositories, solutions, syrups, emulsions or dispersible powders. Corresponding tablets may be obtained for example by mixing the active substance(s) with known excipients, for example inert diluents, such as calcium carbonate, calcium phosphate or lactose, disintegrants such as maize starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g. with the addition of preservatives such as p-hydroxybenzoates, or stabilisers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

The inhalable powders which may be used according to the invention may contain the active substance according to the invention either on its own or in admixture with suitable physiologically acceptable excipients.

If the active substances according to the invention are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose), oligo- and polysaccharides (e.g. dextrans), polyalcohols (e.g. sorbitol, mannitol, xylitol), salts (e.g. sodium chloride, calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. In some cases it may seem appropriate to add finer excipient fractions with an average particle size of 1 to 9 μm to the excipient mentioned above. These finer excipients are also selected from the group of possible excipients listed hereinbefore. Finally, in order to prepare the inhalable powders according to the invention, micronised active substances according to the invention, preferably with an average particle size of 0.5 to 10 μm, more preferably from 1 to 5 μm, are added to the excipient mixture. Processes for producing the inhalable powders according to the invention by grinding and micronising and finally mixing the ingredients together are known from the prior art.

The inhalable powders according to the invention may be administered using inhalers known from the prior art.

Inhalation aerosols containing propellant gas according to the invention may contain active substances according to the invention dissolved in the propellant gas or in dispersed form. The propellant gases which may be used to prepare the inhalation aerosols are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The above-mentioned propellant gases may be used on their own or in admixture. Particularly preferred propellant gases are halogenated alkane derivatives selected from TG134a and TG227 and mixtures thereof.

The propellant-driven inhalation aerosols may also contain other ingredients such as co-solvents, stabilisers, surfactants, antioxidants, lubricants and pH adjusters. All these ingredients are known in the art.

The propellant-driven inhalation aerosols according to the invention mentioned above may be administered using inhalers known in the art (MDIs=metered dose inhalers).

Moreover, the active substances according to the invention may be administered in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is preferably up to 70 percent by volume, more particularly up to 60 percent by volume and most preferably up to 30 percent by volume. The remainder of the volume is made up of water. The solutions or suspensions containing the active substance according to the invention are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

The addition of editic acid (EDTA) or one of the known salts thereof, sodium edetate, as stabiliser or complexing agent may optionally be omitted in these formulations. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the active substance according to the invention, only benzalkonium chloride and sodium edetate. In another preferred embodiment, no sodium edetate is present.

A therapeutically effective daily dose is between 1 and 2000 mg, preferably 10-500 mg per adult.

The Examples which follow illustrate the present invention without restricting its scope:

Examples of Pharmaceutical Formulations

| A) Tablets | per tablet | |
|---|---|---|
| active substance | 100 | mg |
| lactose | 140 | mg |
| maize starch | 240 | mg |
| polyvinylpyrrolidone | 15 | mg |
| magnesium stearate | 5 | mg |
| | 500 | mg |

The finely ground active substance, lactose and some of the corn starch are mixed together. The mixture is screened, then moistened with a solution of polyvinylpyrrolidone in water, kneaded, granulated while wet and dried. The granulate, the rest of the corn starch and the magnesium stearate are screened and mixed together. The mixture is compressed to form tablets of a suitable shape and size.

| B) Tablets | per tablet | |
|---|---|---|
| active substance | 80 | mg |
| corn starch | 190 | mg |
| lactose | 55 | mg |
| microcrystalline cellulose | 35 | mg |
| polyvinylpyrrolidone | 15 | mg |
| sodium-carboxymethyl starch | 23 | mg |
| magnesium stearate | 2 | mg |
| | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

| C) Coated tablets | per coated tablet | |
|---|---|---|
| Active substance | 5 | mg |
| Corn starch | 41.5 | mg |
| Lactose | 30 | mg |
| Polyvinylpyrrolidone | 3 | mg |
| Magnesium stearate | 0.5 | mg |
| | 80 | mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in a known manner with a covering consisting essentially of sugar and talc. The finished coated tablets are polished with wax

| D) Capsules | per capsule | |
|---|---|---|
| Active substance | 50 | mg |
| Corn starch | 268.5 | mg |
| Magnesium stearate | 1.5 | mg |
| | 320 | mg |

The substance and corn starch are mixed and moistened with water. The moist mass is screened and dried. The dry granules are screened and mixed with magnesium stearate. The finished mixture is packed into size 1 hard gelatine capsules.

| E) Ampoule solution | | |
|---|---|---|
| active substance | 50 | mg |
| sodium chloride | 50 | mg |
| water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

| F) Suppositories | | |
|---|---|---|
| Active substance | 50 | mg |
| Solid fat | 1650 | mg |
| | 1700 | mg |

The hard fat is melted. At 40° C. the ground active substance is homogeneously dispersed. It is cooled to 38° C. and poured into slightly chilled suppository moulds.

| G) Oral suspension | | |
|---|---|---|
| active substance | 50 | mg |
| hydroxyethylcellulose | 50 | mg |
| sorbic acid | 5 | mg |
| sorbitol (70%) | 600 | mg |
| glycerol | 200 | mg |
| flavouring | 15 | mg |
| water ad | 5 | ml |

Distilled water is heated to 70° C. Hydroxyethyl-cellulose is dissolved therein with stirring. After the addition of sorbitol solution and glycerol the mixture is cooled to ambient temperature. At ambient temperature, sorbic acid, flavouring and substance are added. To eliminate air from the suspension it is evacuated with stirring.

and 50 mg of active substance.

| H) Metered-dose aerosol (suspension) | | |
|---|---|---|
| active substance | 0.3 | wt. % |
| sorbitolan trioleate | 0.6 | wt. % |
| HFA134A:HFA227 2:1 | 99.1 | wt. % |

The suspension is transferred into a conventional aerosol container with a metering valve. Preferably, 50 μl of suspension are delivered per spray. The active substance may also be metered in higher doses if desired.

| I) Metered-dose aerosol (solution) | | |
|---|---|---|
| active substance | 0.3 | wt. % . % |
| abs. ethanol | 20 | wt. % |
| aqueous HCl 0.01 mol/l | 2.0 | wt. % |
| HFA134A | 77.7 | wt. % |

The solution is produced in the usual way by mixing the individual ingredients together.

| J) Inhalable powder | | |
|---|---|---|
| active substance | 80 | μg |
| lactose monohydrate | ad 10 | mg |

The powder for inhalation is produced in the usual way by mixing the individual ingredients together.

1. A compound of the formula (I), 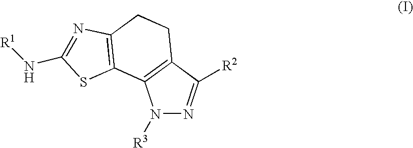
wherein
R¹ denotes hydrogen, CO—CH₃, CO—CH₂—R⁴, CO—CHMe-R⁴, CO—OR⁴, CO-—SR⁴, CO—NH₂, CO—NHR⁴;
R² denotes a group selected from among C₃₋₆-cycloalkyl, C₁₋₄-alkyl-C₃₋₆-cycloalkyl, C₂₋₄-alkenyl-C₃₋₆-cycloalkyl, C₂₋₄-alkynyl-C₃₋₆-cycloalkyl, C₅₋₆-cycloalkenyl, C₁₋₆-alkyl-C₅₋₆-cycloalkenyl, C₂₋₄-alkenyl-C₅₋₆-cycloalkenyl, C₂₋₄-alkynyl-C₅₋₆-cycloalkenyl, C₅₋₆-cycloalkynyl, C₁₋₆-alkyl-C₅₋₆-cycloalkynyl, C₂₋₄-alkenyl-C₅₋₆-cycloalkynyl- and C₂₋₄-alkynyl-C₅₋₆-cycloalkynyl, which may optionally be substituted by one or two of the groups CH₃, F, OCH₃, OH or NH₂;
R³ denotes a group selected from among C₆-C₁₄-aryl, C₁₋₆-alkyl-C₆-C₁₄-aryl, C₂₋₆-alkenyl-C₆-C₁₄-aryl, C₂₋₆-alkynyl-C₆-C₁₄-aryl, C₅-C₁₀-heteroaryl, C₁₋₁₂-alkyl-C₅-C₁₀-heteroaryl, C₃₋₁₂-alkenyl-C₅-C₁₀-heteroaryl, C₃₋₁₂-alkynyl-C₅-C₁₀-heteroaryl, C₃₋₆-cycloalkyl, C₁₋₆-alkyl-C₃₋₆-cycloalkyl, C₂₋₄-alkenyl-C₃₋₆-cycloalkyl, C₂₋₄-alkynyl-C₃₋₆-cycloalkyl, C₅₋₆-cycloalkenyl, C₁₋₆-alkyl-C₅₋₆-cycloalkenyl, C₂₋₄-alkenyl-C₅₋₆-cycloalkenyl, C₂₋₄-alkynyl-C₅₋₆-cycloalkenyl, C₅₋₆-cycloalkynyl, C₁₋₆-alkyl-C₅₋₆-cycloalkynyl, C₂₋₄-alkenyl-C₅₋₆-cycloalkynyl- and C₂₋₄-alkynyl-C₅₋₆-cycloalkynyl, which may optionally be substituted by a group R⁵ and up to three groups R⁶;
or optionally substituted 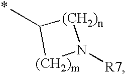
wherein
n, m, independently of one another denote 1 or 2;
R⁴ denotes an optionally substituted group selected from among C₁₋₄-alkyl, C₂₋₁₀-alkenyl, C₂₋₁₀-alkynyl, C₃₋₆-cycloalkyl-C₁₋₄-alkyl, C₃₋₆-cycloalkyl-C₃₋₁₀-alkenyl, C₃₋₆-cycloalkyl-C₃₋₁₀-alkynyl, C₆-C₁₄-aryl, C₆-C₁₄-aryl-C₁₋₄-alkyl, C₅-C₁₀-heteroaryl, C₅-C₁₀-heteroaryl-C₁₋₄-alkyl- and haloalkyl;
R⁵ denotes CONR⁸R⁹, NR⁸COR⁹, NR⁸R⁹, OR⁹, —C₁₋₄-alkyl-CONR⁸R⁹;
R⁶ which may be identical or different, denote F, Cl, Br, OH, CN, CF₃, CHF₂ or an optionally substituted group selected from among —O-C₁₋₃-alkyl, —O—C₃₋₄-alkenyl, -O—C₃₋₄-alkynyl, C₁₋₃-alkyl, C₂₋₆-alkenyl and C₂₋₃-alkynyl, C₃₋₆-cycloalkyl-C₁₋₄-alkyl, C₃₋₆-cycloalkyl-C₂₋₄-alkenyl, C₃₋₆-cycloalkyl-C₂₋₄-alkynyl, C₅₋₆-cycloalkenyl-C₁₋₄-alkyl, C₅₋₆-cycloalkenyl-C₃₋₁₀-alkenyl, C₅₋₆-cycloalkenyl-C₂₋₄-alkynyl, C₆-C₁₄-aryl-C₁₋₄-alkyl, C₆-C₁₄-aryl-C₂₋₄-alkenyl-, C₆-C₁₄-aryl-C₂₋₄-alkynyl, C₅-C₁₀-heteroaryl-C₁₋₄-alkyl, C₅-C₁₀-heteroaryl-C₂₋₄-alkenyl- and C₅-C₁₀-heteroaryl-C₂₋₄-alkynyl;
R⁷ denotes hydrogen, COR⁹, CONR⁸R⁹ or
a group selected from among C₁₋₁₀-alkyl, C₃₋₁₀-alkenyl, C₃₋₁₀-alkynyl, C₃₋₆-cycloalkyl-C₁₋₄-alkyl, C₃₋₆-cycloalkyl-C₃₋₁₀-alkenyl, C₃₋₆-cycloalkyl-C₃₋₁₀-alkynyl, C₅₋₆-cycloalkenyl-C₁₋₄-alkyl, C₅₋₆-cycloalkenyl-C₃₋₁₀-alkenyl, C₅₋₆-cycloalkenyl-C₃₋₁₀-alkynyl, C₆-C₁₄-aryl, C₁₋₁₀-alkyl-C₆-C₁₄-aryl, C₂₋₁₀-alkenyl-C₆-C₁₄-aryl-, C₂₋₁₀-alkynyl-C₆-C₁₄-aryl, C₅-C₁₀-heteroaryl, C₁₋₁₂-alkyl-C₅-C₁₀-heteroaryl, C₃₋₁₂-alkenyl-C₅-C₁₀-heteroaryl- and C₃₋₁₂-alkynyl-C₅-C₁₀-heteroaryl, which may optionally be substituted by a group R¹⁴ and with a group R¹³ may be substituted;
R⁸ denotes hydrogen or
an optionally substituted group selected from among C₁₋₁₀-alkyl, C₃₋₁₀-alkenyl, C₃₋₁₀-alkynyl, C₃₋₆-cycloalkyl-C₁₋₄-alkyl, C₃₋₆-cycloalkyl-C₃₋₁₀-alkenyl, alkenyl, C₃₋₆-cycloalkyl-C₃₋₁₀-alkynyl, C₅₋₆-cycloalkenyl-C₁₋₄-alkyl, C₅₋₆-cycloalkenyl-C₃₋₁₀-alkenyl, C₅₋₆-cycloalkenyl-C₃₋₁₀-alkynyl, C₆-C₁₄-aryl-C₁₋₄-alkyl, C₆-C₁₄-aryl-C₃₋₁₀-alkenyl- and C₆-C₁₄-aryl-C₃₋₁₀-alkynyl, C₅-C₁₀-heteroaryl, C₅-C₁₀-heteroaryl-C₁₋₄-alkyl, C₅-C₁₀-heteroaryl-C₁₋₄-alkenyl, C₅-C₁₀-heteroaryl-C₁₋₄-alkynyl, C₁₋₄-alkyl-O—C₂₋₄-alkyl, C₁₋₄-alkyl-O-C₄₋₆-alkenyl- and C₁₋₄-alkyl-O-C₄₋₆-alkynyl-;
R⁹ denotes hydrogen or
an optionally substituted group selected from among C₁₋₁₂-alkyl, C₃₋₁₂-alkenyl, C₃₋₁₂-alkynyl, C₃₋₆-cycloalkyl-C₁₋₁₂-alkyl, C₃₋₆-cycloalkyl-C₃₋₁₂-alkenyl, C₃₋₆-cycloalkyl-C₃₋₁₂-alkynyl, C₅₋₆-cycloalkenyl-C₁₋₄-alkyl, C₅₋₆-cycloalkenyl-C₃₋₁₀-alkenyl, C₅₋₆-cycloalkenyl-C₃₋₁₀-alkynyl, C₆-C₁₄-aryl-C₁₋₁₂-alkyl, C₆-C₁₄-aryl-C₃₋₁₂-alkenyl, C₆-C₁₄-aryl-C₃₋₁₂-alkynyl, C₆-C₁₄-aryl, C₁₋₁₂-alkyl-C₆-C₁₄-aryl, C₂₋₁₂-alkenyl-C₆-C₁₄-aryl, C₂₋₁₂-alkynyl-C₆-C₁₄-aryl, C₅-C₁₀-heteroaryl, C₅-C₁₀-heteroaryl-C₁₋₁₂-alkyl, C₅-C₁₀-heteroaryl-C₃₋₁₂-alkenyl, C₅-C₁₀-heteroaryl-C₃₋₁₂-alkynyl, C₃₋₈-cycloalkyl, C₅₋₈-cycloalkenyl, NR¹¹R¹²—C₃₋₈-cycloalkyl, NR¹¹R¹²—C₅₋₈-cycloalkenyl- and NR¹¹R¹²—C₅₋₈-cycloalkynyl or
an optionally substituted C₃₋₈-heterocycloalkyl-(CH₂)_{q} group, containing at least one NR¹⁰ group in the 3- to 8-membered heterocyclic group, or
R⁸ and R⁹ together form a saturated or unsaturated 4- to 7-membered alkyl bridge which optionally contains an O atom or an S(O)ₚ group,
wherein p, q independently of one another denote 0, 1 or 2; or
NR⁸R⁹ denotes a 5- to 6-membered heterocyclic group, optionally containing a further N atom and optionally substituted by a group selected from among R¹⁰, NR¹¹R¹² and NR¹¹R¹²C₁₋₄-alkyl, or
a group 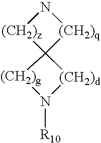
wherein
z, q, g, d independently of one another denote 1 , 2 or 3;
R¹⁰ denotes hydrogen or
an optionally substituted group selected from among C₁₋₁₀-alkyl, C₃₋₁₀-alkenyl, C₃₋₁₀-alkynyl, C₃₋₇-cycloalkyl-C₁₋₁₀-alkyl, C₃₋₇-cycloalkyl-C₃₋₁₀-alkenyl, C₃₋₇-cycloalkyl-C₃₋₁₀-alkynyl, C₃₋₇-cycloalkyl, C₁₋₆-alkyl-C₃₋₇-cycloalkyl, C₂₋₄-alkenyl-C₃₋₇-cycloalkyl, C₂₋₄-alkynyl-C₃₋₇-cycloalkyl, tetrahydropyranyl and (NR⁴)₂CH—C₁₋₁₀-alkyl,
R¹¹, R¹² which may be identical or different denote hydrogen or
an optionally substituted group selected from among C₁₋₁₀-alkyl, C₃₋₁₀-alkenyl, C₃₋₁₀-alkynyl, C₃₋₆-cycloalkyl-C₁₋₄-alkyl- and C₃₋₆-cycloalkyl or
R¹¹ and R¹² together form a 4- to 7-membered alkyl chain which optionally contains a heteroatom;
R¹³ denotes F, Cl, Br, OH, CN, CF₃, CHF₂ or C₁₋₄-alkyl-O—;
R¹⁴ denotes NR¹¹R¹² or an optionally substituted C₃₋₈-heterocycloalkyl-(CH₂)_{q} group, containing at least one NR¹⁰ group in the 3- to 8-membered heterocyclic group, or
R¹³ and R¹⁴ together form a saturated or unsaturated 4- to 7-membered alkyl bridge which optionally contains an O atom or an S(O)ₚ group;
optionally in the form of the tautomers, the racemates, the enantiomers, the diastereomers and the mixtures thereof, as well as optionally the pharmacologically acceptable acid addition salts, solvates and hydrates thereof.

2. The compound according to claim 1, wherein
R¹ and R³ to R¹⁴ may have the meanings specified and
R² denotes an optionally substituted group, with one or two of the groups CH₃, F, OCH₃, OH or NH₂, selected from among C₃₋₆-cycloalkyl, C₁₋₆-alkyl-C₃₋₆-cycloalkyl- and C₂₋₄-alkenyl-C₃₋₆-cycloalkyl.

3. The compound according to claim 2, wherein
R¹, R² and R⁴ to R¹⁴ may have the meanings specified and
R³ denotes a group selected from among phenyl and C₅₋₆-cycloalkyl, which may optionally be substituted by a group R⁵ and up to three groups R⁶,
or optionally substituted 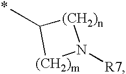
wherein
n, m, independently of one another denote 1 or 2.

4. The compound according to claim 3, wherein
R¹ to R⁷ and R¹⁰ to R¹⁴ may have the meanings specified and
R⁸ denotes hydrogen or
an optionally substituted group selected from among C₁₋₁₀-alkyl, C₃₋₁₀-alkenyl, C₃₋₁₀-alkynyl and C₁₋₄-alkyl-O—C₂₋₄-alkyl;
R⁹ denotes hydrogen or
an optionally substituted group selected from among C₁₋₁₂-alkyl, C₃₋₁₂-alkenyl, C₃₋₁₂-alkynyl, C₃₋₆-cycloalkyl-C₁₋₁₂-alkyl, C₆-C₁₄-aryl, C₁₋₁₂-alkyl-C₆-C₁₄-aryl, C₂₋₁₂-alkenyl-C₆-C₁₄-aryl, C₂₋₁₂-alkynyl-C₆-C₁₄-aryl, C₅-C₁₀-heteroaryl, C₅-C₁₀-heteroaryl-C₁₋₁₂-alkyl, C₅-C₁₀-heteroaryl-C₃₋₁₂-alkenyl, C_{5-C}₁₀-heteroaryl-C₃₋₁₂-alkynyl, C₃₋₈-cycloalkyl, C₅₋₈-cycloalkenyl and NR¹¹R¹²—C₃₋₈-cycloalkyl, or
an optionally substituted C₃₋₈-heterocycloalkyl-(CH₂)_{q}— containing at least one NR¹⁰ group in the 3- to 8-membered heterocyclic group, or
R⁸ and R⁹ together form a saturated or unsaturated 4- to 7-membered alkyl bridge which optionally contains an O atom or an S(O)ₚ group,
wherein p, q independently of one another denote 0, 1 or 2; or
NR⁸R⁹ denotes a 5- to 6-membered heterocyclic group, optionally containing a further N atom and optionally substituted by a group selected from among R¹⁰, NR¹¹R¹² and NR¹¹R¹²C₁₋₄-alkyl, or
a group 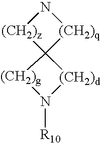
wherein
z, q, g, d independently of one another denote 1, 2 or 3.

5. The compound according to claim 4, wherein
R¹ to R⁷ and R¹⁰ to R¹⁴ may have the meanings specified and
R⁸ denotes hydrogen or
an optionally substituted group selected from among C₁₋₁₀-alkyl, C₃₋₁₀-alkenyl, C₃₋₁₀-alkynyl and C₁₋₄-alkyl-O—C₁₋₄-alkyl,
R⁹ denotes hydrogen or
an optionally substituted group selected from among C₁₋₁₂-alkyl, C₃₋₁₂-alkenyl, C₃₋₁₂-alkynyl, C₃₋₆-cycloalkyl-C₁₋₁₂-alkyl, C₆-C₁₄-aryl, C₁₋₁₂-alkyl-C₆-C₁₄-aryl, C₂₋₁₂-alkenyl-C₆-C₁₄-aryl, C₂₋₁₂-alkynyl-C₆-C₁₄-aryl, C₅-C₁₀-heteroaryl, C₅-C₁₀-heteroaryl-C₁₋₁₂-alkyl, C₅-C₁₀-heteroaryl-C₃₋₁₂-alkenyl, C₅-C₁₀-heteroaryl-C₃₋₁₂-alkynyl, C₃₋₈-cycloalkyl, C₅₋₈-cycloalkenyl and NR¹¹ R¹²—C₃₋₈-cycloalkyl, or
an optionally substituted group selected from among the general formulae (A1) to (A12) 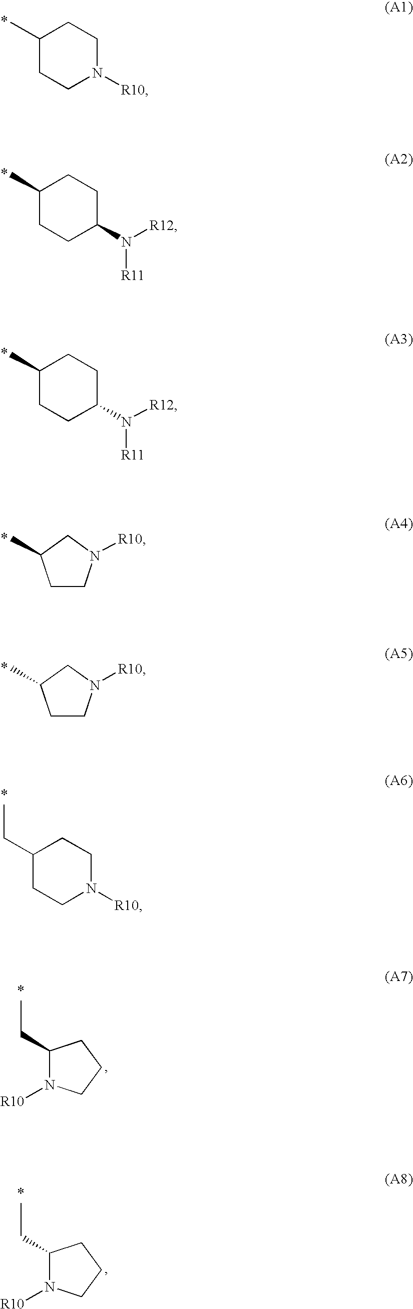 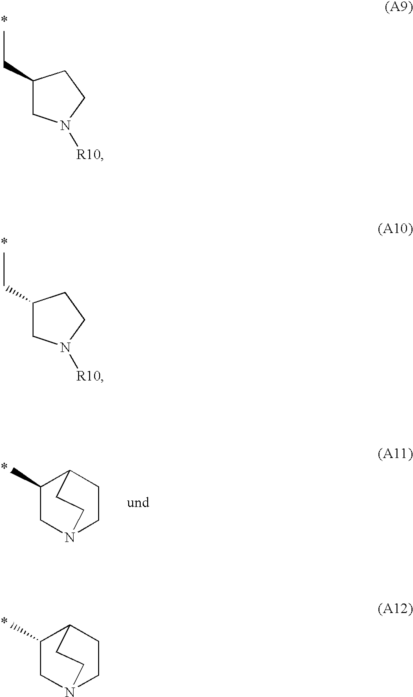
or
R⁸ and R⁹ together form a saturated or unsaturated 4- to 7-membered alkyl bridge which optionally contains an O atom or an S(O)ₚ group,
wherein p, q independently of one another denote 0, 1 or 2; or
NR⁸R⁹ denotes an optionally substituted group selected from among the general formulae (B1) to (B8) 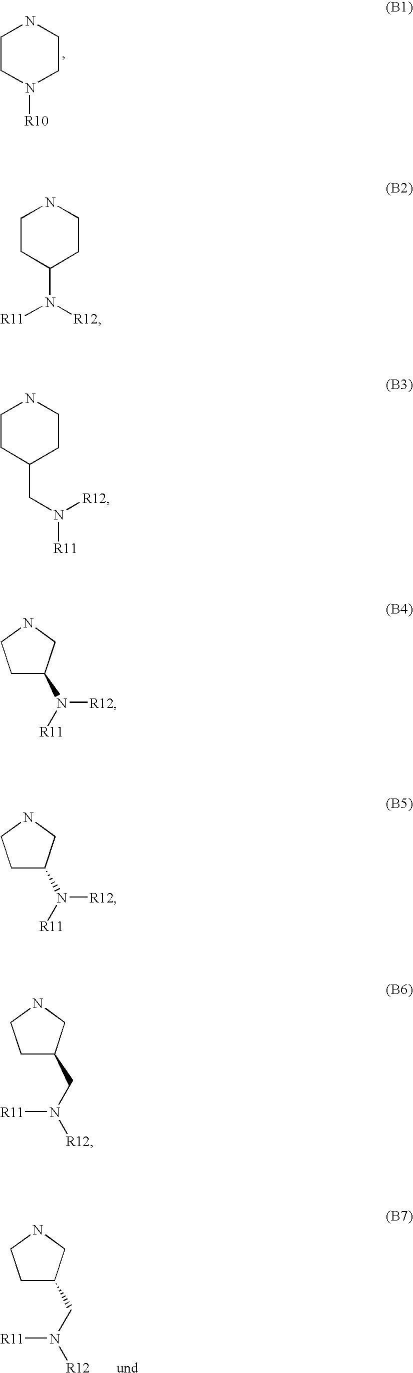 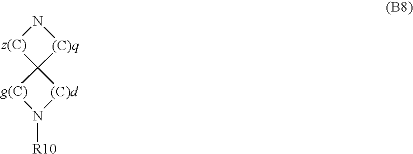
wherein
z, q, g, d independently of one another denote 1, 2 or 3.

6. The compound according to claim 5, wherein
R¹ to R ⁸ and R¹⁰ to R¹² may have the meanings specified and
R⁷ denotes COR⁹ or CONR⁸R⁹.

7. The compound according to claim 6, wherein
R¹ to R ⁵ and R⁷ to R¹⁴ may have the meanings specified and
R⁶ which may be identical or different, denote F, Cl, CF₃, or an optionally substituted group —O—C₁₋₃-alkyl or C₁₋₃-alkyl.

8. The compound according to claim 7, wherein
R⁴ to R⁶ and R¹⁰ to R¹² may have the meanings specified and
R¹ denotes CO—CH₃, CO—CH₂—R⁴;
R² denotes cyclopropyl, optionally substituted by one or two of the groups CH₃, F, OCH₃, OH or NH₂;
R³ denotes optionally substituted 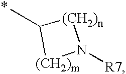
wherein
n, m, independently of one another denote 1 or 2;
R⁷ denotes hydrogen, COR⁹, or CONR⁸R⁹,
R⁸ denotes hydrogen or C₁₋₁₀-alkyl,
R⁹ denotes hydrogen or
an optionally substituted group selected from among C₃₋₈-cycloalkyl and NR¹¹R¹²—C₃₋₈-cycloalkyl, or
an optionally substituted group selected from among the general formulae (A1) to (A12) 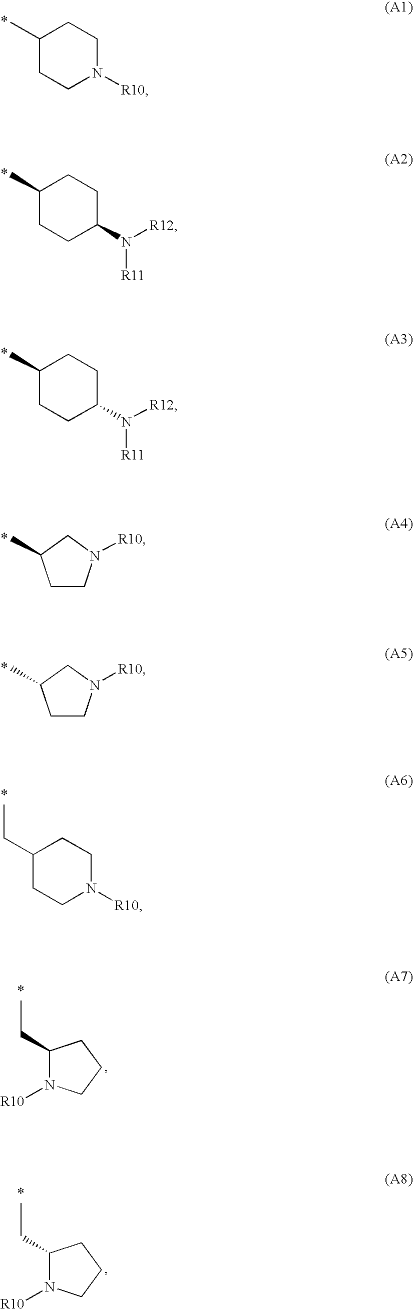 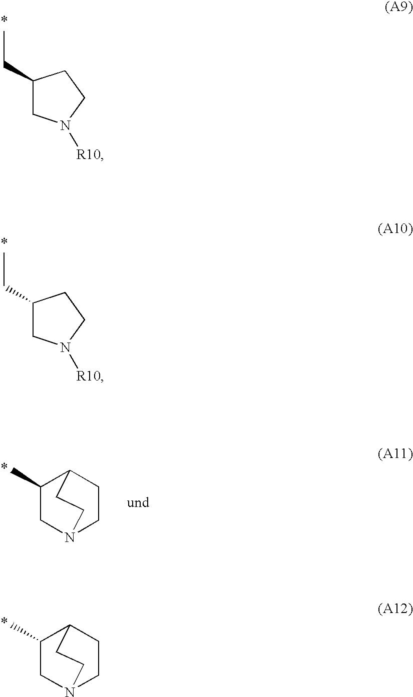
or
NR⁸R⁹ denotes a 5- to 6-membered heterocyclic group, containing 1 to 3 N-atoms, optionally substituted by a group selected from among R¹⁰, NR¹¹R¹² and NR¹¹R¹²C₁₋₄-alkyl.

9. The compound according to claim 7, wherein
R⁴ to R⁶ and R¹⁰ to R¹² may have the meanings specified and
R¹ denotes CO—CH₃, CO—CH₂—R⁴;
R² denotes C₃₋₆-cycloalkyl, which may optionally be substituted by one or two of the groups CH₃, F, OCH₃, OH or NH₂;
R³ denotes a group selected from among phenyl and C₅₋₆-cycloalkyl, which may optionally be substituted by one R⁵ and up to three R⁶;
R⁵ denotes NR⁸R⁹, CONR⁸R⁹, NR⁸COR⁹ or —C₁₋₄-alkyl-CONR⁸R⁹;
R⁶ which may be identical or different, denote F, Cl, Br, CF₃ or an optionally substituted group selected from among —O—C₁₋₃-alkyl, C₁₋₃-alkyl, C₃₋₆-cycloalkyl-C₁₋₄-alkyl- and C₆-C₁₄-aryl-C₁₋₄-alkyl,
R⁸ denotes hydrogen or
optionally substituted C₁₋₁₀-alkyl;
R⁹ denotes hydrogen or
an optionally substituted group selected from among C₁₋₁₂-alkyl, C₃₋₆-cycloalkyl-C₁₋₁₂-alkyl, C₆-C₁₄-aryl, C₁₋₁₂-alkyl-C₆-C₁₄-aryl, C₅-C₁₀-heteroaryl, C₅-C₁₀-heteroaryl-C₁₋₁₂-alkyl, C₃₋₈-cycloalkyl, C₅₋₈-cycloalkenyl and NR¹¹R¹²—C₃₋₈-cycloalkyl, or
an optionally substituted group selected from among the general formulae (A1) to (A12) 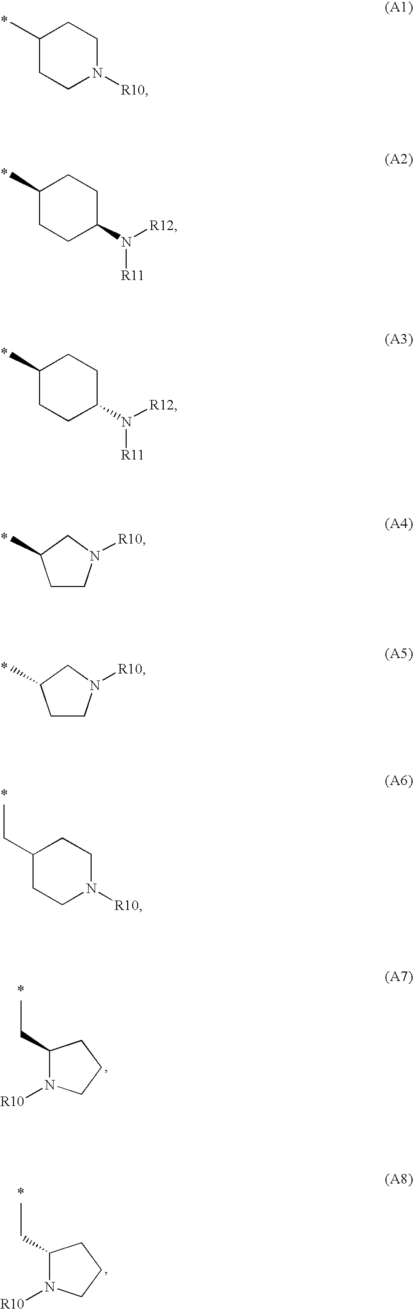 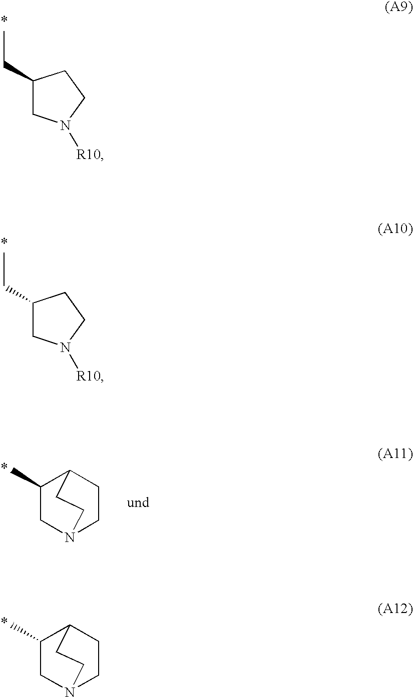
R⁸ and R⁹together form a saturated or unsaturated 4- to 7-membered alkyl bridge which optionally contains an O atom or an S(O)ₚ group,
wherein p, q independently of one another denote 0, 1 or 2; or
NR⁸R⁹ denotes an optionally substituted group selected from among the general formulae (B1) to (B8) 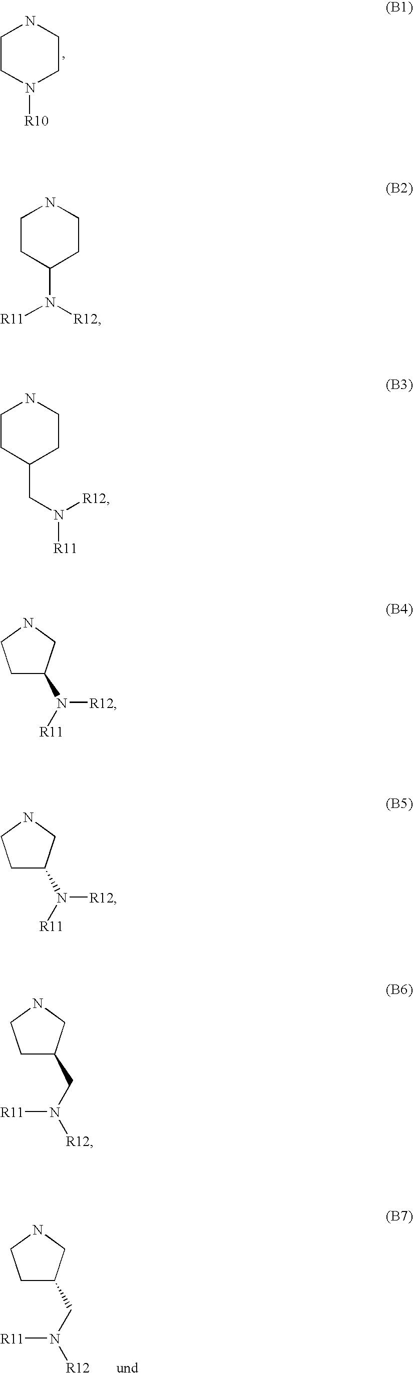 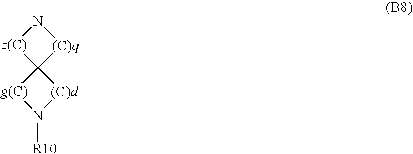
wherein
z, q, g, d independently of one another denote 1 , 2 or 3.
R¹⁰ denotes hydrogen or
an optionally substituted group selected from among C₁₋₁₀-alkyl, C₃₋₇-cycloalkyl-C₁₋₁₀-alkyl, C₃₋₇-cycloalkyl, C₁₋₆-alkyl-C₃₋₇-cycloalkyl, tetrahydropyranyl and (NR⁴)₂CH—C₁₋₁₀-alkyl.

10. A method of treating a disease or condition chosen from chronic bronchitis, acute bronchitis, bronchitis caused by bacterial or viral infection or fungi or helminths, allergic bronchitis, toxic bronchitis, chronic obstructive bronchitis (COPD), asthma (intrinsic or allergic), paediatric asthma, bronchiectasis, allergic alveolitis, allergic or non-allergic rhinitis, chronic sinusitis, cystic fibrosis or mucoviscidosis, alpha-1-antitrypsin deficiency, cough, pulmonary emphysema, interstitial lung diseases, alveolitis, hyperreactive airways, nasal polyps, pulmonary oedema, pneumonitis of different origins, e.g. radiation-induced or caused by aspiration, or infectious pneumonitis, collagenoses such as lupus erythematodes, systemic sclerodermy, sarcoidosis and Boeck's disease comprising administering a therapeutically effective amount of a compound according to claim 1.

11. A method of treating a disease or condition chosen from psoriasis, contact dermatitis, atopic dermatitis, alopecia areata (circular hair loss), erythema exsudativum multiforme (Stevens-Johnson Syndrome), dermatitis herpetiformis, sclerodermy, vitiligo, nettle rash (urticaria), lupus erythematodes, follicular and surface pyodermy, endogenous and exogenous acne, acne rosacea and other inflammatory and allergic or proliferative skin diseases comprising administering a therapeutically effective amount of a compound according to claim 1.

12. A method of treating a disease or condition chosen from inflammation of the conjunctiva (conjunctivitis) of various kinds, such as e.g. caused by infection with fungi or bacteria, allergic conjunctivitis, irritable conjunctivitis, drug-induced conjunctivitis, keratitis and uveitis comprising administering a therapeutically effective amount of a compound according to claim 1.

13. A method of treating a disease or condition chosen from allergic rhinitis, allergic sinusitis and nasal polyps comprising administering a therapeutically effective amount of a compound according to claim 1.

14. A method of treating a disease or condition chosen from Crohn's disease, ulcerative colitis, systemic lupus erythematodes, chronic hepatitis, multiple sclerosis, rheumatoid arthritis, psoriatic arthritis, osteoarthritis, rheumatoid spondylitis comprising administering a therapeutically effective amount of a compound according to claim 1.

15. A method of treating a disease or condition chosen from glomerulonephritis, interstitial nephritis and idiopathic nephrotic syndrome comprising administering a therapeutically effective amount of a compound according to claim 1.

16. A pharmaceutical composition comprising a pharmaceutically effective amount of a compound according to claim 1.

17. The pharmaceutical composition according to claim 16 for inhalative administration.

18. The pharmaceutical composition according to claim 16 for oral administration.

19. The pharmaceutical composition according to claim 16, comprising as a further active substance, one or more compounds which are selected from the categories of the betamimetics, anticholinergics, corticosteroids, other PDE4-inhibitors, LTD4-antagonists, EGFR-inhibitors, dopamine agonists, H1-antihistamines, PAF-antagonists and PI3-kinase inhibitors or double or triple combinations thereof.

20. A process for preparing compounds of the formula (I), 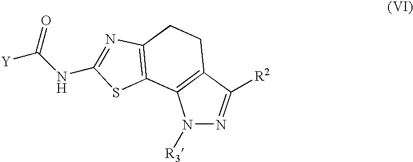
wherein
the group R² may have the meanings specified,
R₃′ denotes an optionally substituted group, selected from among 4-PhCOOMe, 4-PhNO₂, 4-piperidyl, cis/trans-4-alkoxycarbonylcylohexyl and 4-methoxycarbonyl-methy-phenyl,
and
Y=C₁-C₄-alkyl or —S—C₁-C₄-alkyl,
comprising
(a) reacting a compound of formula (II) 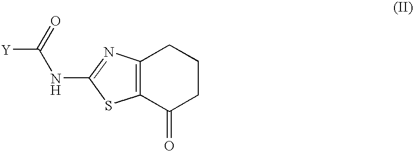
with a compound of formula (III) 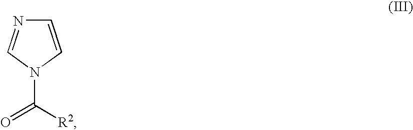
wherein R² may have the meaning specified,
and
(b) reacting the compound of formula (IV) 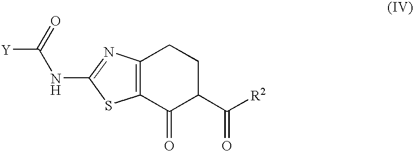
resulting from step (a) with a compound of formula (V) 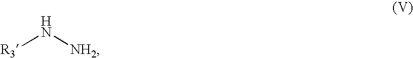
wherein
R₃′ may have the meaning specified,
cyclising to obtain the compound of formula (VI).

21. A process for preparing compounds of the formula (Ib) 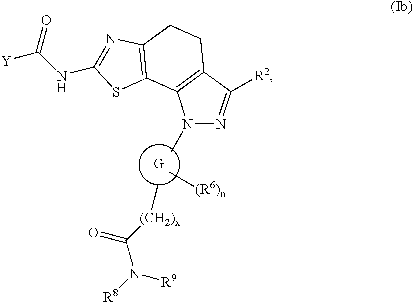
wherein
R², R⁶, R⁸ and R⁹ may have the meanings specified,
G denotes phenyl or cyclohexyl, and
X denotes 0 or 1,
comprising
(a) reacting a compound of formula (VIa) 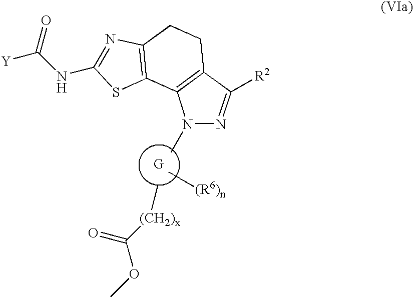
wherein
R², R⁶ and Y may have the meanings specified,
with an alkali metal hydroxide to form a compound of formula (VII) 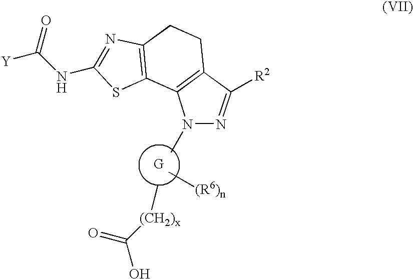
and
(b) reacting the compound of formula (VII) resulting from step (a) with a compound of formula (VIII) 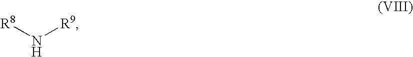
wherein R⁸ and R⁹ may have the meanings specified,
to form a compound of formula (Ib).

22. A process for preparing compounds of the formula (Ic) or (Id) 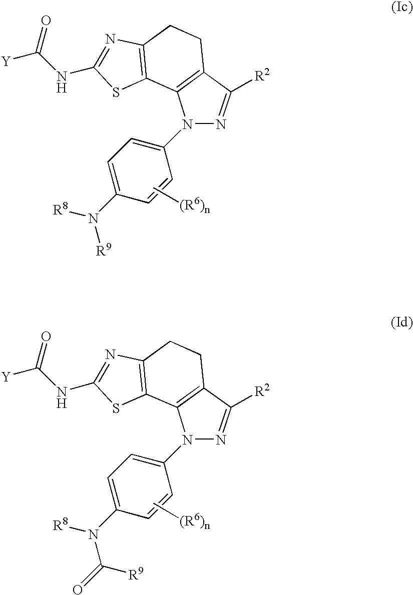
wherein
R², R⁶, R⁸, R⁹ and Y may have the meanings specified,
comprising
(a) reducing a compound of formula (VIb) 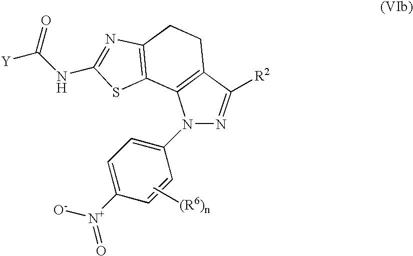
to form a compound of formula (IX) 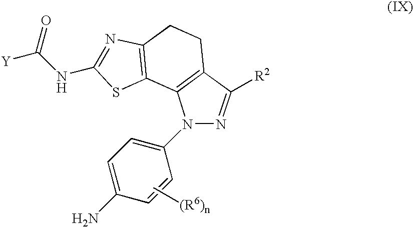
and
reacting by reductive amination the compound of formula (IX) resulting from step (a) to form a compound of formula (Ic) or (Id).

23. A process for preparing compounds of the formula (Ie), (If) or (Ig) 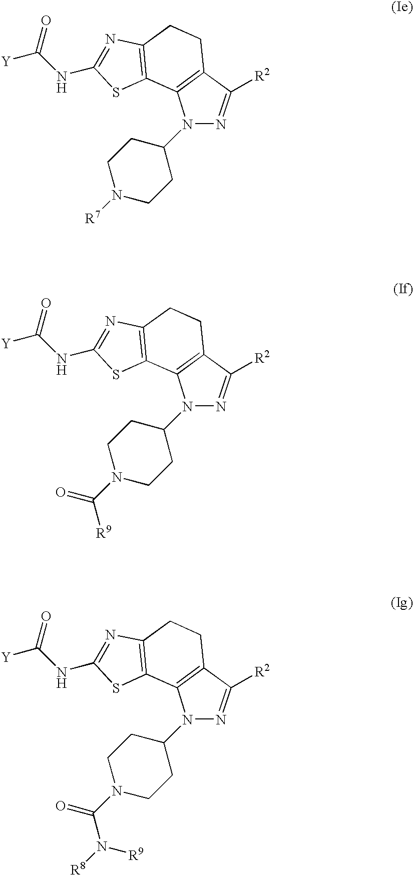
wherein
R², R⁷, R⁸, R⁹ and Y may have the meanings specified,
comprising
reacting by reductive amination a compound of formula (VIc) 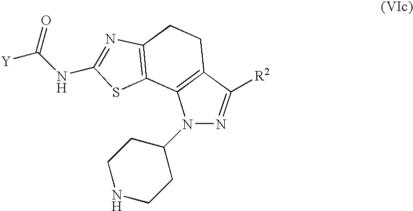
to form a compound of formula (Ie), (If) or (Ig).

24. A compound of the formula (VI) 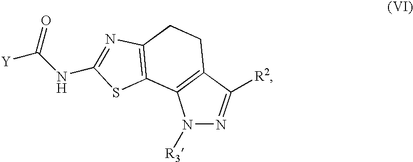
wherein R² denotes a group selected from among C₃₋₆-cycloalkyl, C₁₋₄-alkyl-C₃₋₆-cycloalkyl, C₂₋₄-alkenyl-C₃₋₆-cycloalkyl, C₂₋₄-alkynyl-C₃₋₆-cycloalkyl, C₅₋₆-cycloalkenyl, C₁₋₆-alkyl-C₅₋₆-cycloalkenyl, C₂₋₄-alkenyl-C₅₋₆-cycloalkenyl, C₂₋₄-alkynyl-C₅₋₆-cycloalkenyl, C₅₋₆-cycloalkynyl, C₁₋₆-alkyl-C₅₋₆-cycloalkynyl, C₂₋₄-alkenyl-C₅₋₆-cycloalkynyl- and C₂₋₄-alkynyl-C₅₋₆-cycloalkynyl, which may optionally be substituted by one or two of the groups CH₃, F, OCH₃, OH or NH₂;
R₃′ denotes an optionally substituted group, selected from among 4-PhCOOMe, 4-PhNO₂, 4-piperidyl, cis/trans-4-alkoxycarbonylcylohexyl and 4-methoxycarbonyl-methy-phenyl,
Y=C₁-C₄-alkyl or —S—C₁-C₄-alkyl,
optionally in the form of the tautomers, the racemates, the enantiomers, the diastereomers and the mixtures thereof, as well as optionally the pharmacologically acceptable acid addition salts thereof.

25. A compound of the formula (IX) 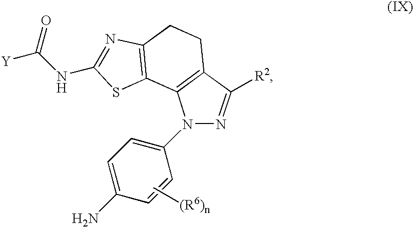
wherein R² denotes a group selected from among C₃₋₆-cycloalkyl, C₁₋₄-alkyl-C₃₋₆-cycloalkyl, C₂₋₄-alkenyl-C₃₋₆-cycloalkyl, C₂₋₄-alkynyl-C₃₋₆-cycloalkyl, C₅₋₆-cycloalkenyl, C₁₋₆-alkyl-C₅₋₆-cycloalkenyl, C₂₋₄-alkenyl-C₅₋₆-cycloalkenyl, C₂₋₄-alkynyl-C₅₋₆-cycloalkenyl, C₅₋₆-cycloalkynyl, C₁₋₆-alkyl-C₅₋₆-cycloalkynyl, C₂₋₄-alkenyl-C₅₋₆-cycloalkynyl- and C₂₋₄-alkynyl-C₅₋₆-cycloalkynyl, which may optionally be substituted by one or two of the groups CH₃, F, OCH₃, OH or NH₂;
R⁶ which may be identical or different, denote F, Cl, Br, OH, CN, CF₃, CHF₂ or an optionally substituted group selected from among —O—C₁₋₃-alkyl, —O—C₃₋₄-alkenyl, —O—C₃₋₄-alkynyl, C₁₋₃-alkyl, C₂₋₆-alkenyl and C₂₋₃-alkynyl, C₃₋₆-cycloalkyl-C₁₋₄-alkyl, C₃₋₆-cycloalkyl-C₂₋₄-alkenyl, C₃₋₆-cycloalkyl-C₂₋₄-alkynyl, C₅₋₆-cycloalkenyl-C₁₋₄-alkyl, C₅₋₆-cycloalkenyl-C₃₋₁₀-alkenyl, C₅₋₆-cycloalkenyl-C₂₋₄-alkynyl, C₆-C₁₄-aryl-C₁₋₄-alkyl, C₆-C₁₄-aryl-C₂₋₄-alkenyl-, C₆-C₁₄-aryl-C₂₋₄-alkynyl, C₅-C₁₀-heteroaryl-C₁₋₄-alkyl, C₅-C₁₀-heteroaryl-C₂₋₄-alkenyl- and C₅-C₁₀-heteroaryl-C₂₋₄-alkynyl;
and Y=C₁-C₄-alkyl or —S—C₁-C₄-alkyl,
optionally in the form of the tautomers, the racemates, the enantiomers, the diastereomers and the mixtures thereof, as well as optionally the pharmacologically acceptable acid addition salts thereof.

26. Compounds of general formula (VII) 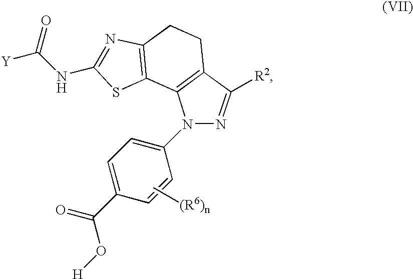
wherein R² denotes a group selected from among C₃₋₆-cycloalkyl, C₁₋₄-alkyl-C₃₋₆-cycloalkyl, C₂₋₄-alkenyl-C₃₋₆-cycloalkyl, C₂₋₄-alkynyl-C₃₋₆-cycloalkyl, C₅₋₆-cycloalkenyl, C₁₋₆-alkyl-C₅₋₆-cycloalkenyl, C₂₋₄-alkenyl-C₅₋₆-cycloalkenyl, C₂₋₄-alkynyl-C₅₋₆-cycloalkenyl, C₅₋₆-cycloalkynyl, C₁₋₆-alkyl-C₅₋₆-cycloalkynyl, C₂₋₄-alkenyl-C₅₋₆-cycloalkynyl- and C₂₋₄-alkynyl-C₅₋₆-cycloalkynyl, which may optionally be substituted by one or two of the groups CH₃, F, OCH₃, OH or NH₂;
R⁶ which may be identical or different, denote F, Cl, Br, OH, CN, CF₃, CHF₂ or an optionally substituted group selected from among —O—C₁₋₃-alkyl, —O—C₃₋₄-alkenyl, —O—C₃₋₄-alkynyl, C₁₋₃-alkyl, C₂₋₆-alkenyl and C₂₋₃-alkynyl, C₃₋₆-cycloalkyl-C₁₋₄-alkyl, C₃₋₆-cycloalkyl-C₂₋₄-alkenyl, C₃₋₆-cycloalkyl-C₂₋₄-alkynyl, C₅₋₆-cycloalkenyl-C₁₋₄-alkyl, C₅₋₆-cycloalkenyl-C₃₋₁₀-alkenyl, C₅₋₆-cycloalkenyl-C₂₋₄-alkynyl, C₆-C₁₄-aryl-C₁₋₄-alkyl, C₆-C₁₄-aryl-C₂₋₄-alkenyl-, C₆-C₁₄-aryl-C₂₋₄-alkynyl, C₅-C₁₀-heteroaryl-C₁₋₄-alkyl, C₅-C₁₀-heteroaryl-C₂₋₄-alkenyl- and C₅-C₁₀-heteroaryl-C₂₋₄-alkynyl;
and Y=C₁-C₄-alkyl or —S—C₁-C₄-alkyl,
optionally in the form of the tautomers, the racemates, the enantiomers, the diastereomers and the mixtures thereof, as well as optionally the pharmacologically acceptable acid addition salts thereof.
